(19) United States
(12) Patent Application Publication
(10) Pub. No.: US 2023/0326554 A1
(43) Pub. Date: Oct. 12, 2023

(54) IDENTIFYING TREATMENT RESPONSE SIGNATURES

(71) Applicant: Rutgers, The State University of New Jersey, New Brunswick, NJ (US)

(72) Inventors: Antonina Mitrofanova, New York, NY (US); Sukanya Panja, Harrison, NJ (US)

(73) Assignee: Rutgers, The State University of New Jersey, New Brunswick, NJ (US)

(21) Appl. No.: 18/297,470

(22) Filed: Apr. 7, 2023

Related U.S. Application Data
(60) Provisional application No. 63/329,135, filed on Apr. 8, 2022.

(51) Int. Cl. G16B 25/10

(52) U.S. Cl. CPC G16B 25/10

(57) ABSTRACT

Provided herein are methods of identifying treatment-response signatures, such as in a subject with prostate cancer (such as a human or veterinary subject). In particular examples, the methods can determine with high accuracy whether a subject is likely to respond to a treatment. The methods can be computer-implemented methods.

### CROSS REFERENCE TO RELATED APPLICATION

This application claims the benefit of U.S. Provisional Application No. 63/329,135, filed Apr. 8, 2022, which is incorporated herein by reference in its entirety.

### ACKNOWLEDGMENT OF GOVERNMENT SUPPORT

This invention was made with government support under grant number R01LM013236-01 awarded by the National Institutes of Health. The government has certain rights in the invention.

### FIELD

The field relates to methods of identifying and using molecular pathways and transcriptional regulatory programs for predicting a response to a therapy in a subject.

### SEQUENCE LISTING INCORPORATION BY REFERENCE

The Sequence Listing is submitted as an XML file in the form of the file named “7213-108205-04_Sequence_Listing.xml” (8,963 bytes), which was created on Apr. 5, 2023 which is incorporated by reference herein.

### BACKGROUND

Since the seminal discovery of the central role of androgen receptor (AR) in prostate tumor growth and progression, androgen-deprivation therapy (ADT, an AR blockade therapy), has been the mainstay of treatment for prostate cancer (PCa). Although approximately 80% of patients initially respond to ADT, a large subset of patients relapse and progress to a more aggressive castration resistant prostate cancer (CRPC). Regardless of the continuous androgen deprivation, patients that develop CRPC retain a high level of AR signaling, which could be due to AR overexpression, AR mutations, or other mechanisms such as upregulation of glucocorticoid receptor that activates a subset of AR target genes. To overcome this problem, more potent AR inhibitors, such as ARSIs (androgen receptor signaling inhibitors), which includes Enzalutamide, Abiraterone, and Apalutamide, have been introduced for CRPC treatment.

While Enzalutamide (an AR signaling inhibitor that can block binding of androgen to androgen receptors with high affinity and can also inhibit AR nuclear translocation and AR binding to DNA) is one of the most commonly used ARSI that has shown to improve patient survival, nearly half of CRPC patients do not respond to it and develop resistance in approximately 8 months to 1.5 years. Unfortunately, CRPC patients that fail Enzalutamide treatment are left with no targeted therapeutic option, and progress to more lethal, metastatic form of CRPC and eventual death. Hence heterogeneity in response to Enzalutamide by CRPC patients pose a critical challenge in prostate cancer management. As a result, the inventors developed Transcriptional-Regulation-2-PATHway (TR-2-PATH) to examine patterns of gene expression across molecular pathways and transcriptional regulatory programs in conditions or diseases to predict patient response to therapeutic options.

### SUMMARY

The Summary is provided to introduce a selection of concepts in a simplified form that are further described below in the Detailed Description. The Summary is not intended to identify key features or essential features of the claimed subject matter, nor is it intended to be used to limit the scope of the claimed subject matter.

There is a critical need for comprehensive analysis of clinical samples to elucidate mechanisms that govern response to treatments, with a special emphasis to identify readily targetable disease drivers to improve clinical outcomes. Transcriptional-Regulation-2-PATHway (TR-2-PATH) was developed herein as a novel framework to reconstruct a de novo, condition- or disease-specific, mechanism-centric regulatory network that connects molecular pathways with their upstream transcriptional regulatory programs. The nodes of the constructed network represent biological mechanisms, such as transcriptional regulatory mechanisms or molecular pathways, making it a comprehensive, multi-layer regulatory view of changes in a condition (e.g., in a cancer cell). Furthermore, connections between nodes represent potential direct or indirect regulatory relationships between upstream transcriptional regulatory program nodes and downstream molecular pathway nodes. Such relationships can constitute candidates for therapeutic targeting.

Provided herein are methods of identifying treatment-response signatures. In some embodiments, the methods include receiving a gene expression dataset from one or more subjects having a condition or disease (such as a cancer, such as a prostate cancer); or receiving a gene expression dataset from one or more cell cultures representative of the condition or disease (such as a cancer, such as a prostate cancer); identifying one or more transcriptional regulatory programs and one or more molecular pathways that are enriched in the gene expression dataset; determining one or more relationships between the one or more transcriptional regulatory programs and the one or more molecular pathways enriched in the gene expression dataset, wherein the determining generates at least one network for the gene expression dataset; and identifying one or more molecular pathways and/or one or more transcriptional regulatory programs in the network that comprise one or more genes that are relatively upregulated or downregulated in a gene expression dataset from a sample that has not received the treatment as compared to a sample that is sensitive to the treatment, relatively upregulated or downregulated in a gene expression dataset from a sample that has not received the treatment as compared to a sample that is resistant to the treatment, or relatively upregulated or downregulated in a gene expression dataset from a sample that is sensitive to the treatment as compared to a sample that exhibits resistance to the treatment, wherein the identifying generates a treatment-response signature. In some examples, the method includes identifying one or more molecular pathways and/or one or more transcriptional regulatory programs in the network that comprise one or more genes that are (a) relatively upregulated in a gene expression dataset from a sample that has not received the treatment as compared to a sample that is sensitive to the treatment, relatively downregulated in a gene expression dataset from a sample that is sensitive to the treatment as compared to a sample that has not received the treatment and/or to a sample that exhibits resistance to the treatment, and relatively upregulated in a gene expression dataset from a sample that exhibits resistance to the treatment as compared to a sample that is sensitive to the treatment; or (b) relatively downregulated in a gene expression dataset from a sample that has not received the treatment as compared to a sample that is sensitive to the treatment, relatively upregulated in a gene expression dataset from a sample that is sensitive to the treatment as compared to a sample that has not received the treatment and/or to a sample that exhibits resistance to the treatment, and relatively downregulated in a gene expression dataset from a sample that exhibits resistance to the treatment as compared to a sample that is sensitive to the treatment. In some embodiments, the treatment-response signature identifies a subject that is predicted to be sensitive to the treatment. In some embodiments, the method includes providing the treatment to the subject if the subject is predicted to be sensitive to the treatment. In some embodiments, the method is a computer-implemented method.

Also provided is a treatment-response signature identification system that includes one or more processors; and memory coupled to the one or more processors, wherein the memory comprises computer-executable instructions causing the one or more processors to perform a process including: receiving a gene expression dataset from one or more subjects having a condition or disease (such as a cancer, such as a prostate cancer); or receiving a gene expression dataset from one or more cell cultures representative of the condition or disease (such as a cancer, such as a prostate cancer); identifying one or more transcriptional regulatory programs and one or more molecular pathways that are enriched in the gene expression dataset; determining one or more relationships between the one or more transcriptional regulatory programs and the one or more molecular pathways enriched in the gene expression dataset, wherein the determining generates at least one network for the gene expression dataset; and identifying one or more molecular pathways and/or one or more transcriptional regulatory programs in the network that comprise one or more genes that are relatively upregulated or downregulated in a gene expression dataset from a sample that has not received the treatment as compared to a sample that is sensitive to the treatment, relatively upregulated or downregulated in a gene expression dataset from a sample that has not received the treatment as compared to a sample that is resistant to the treatment, or relatively upregulated or downregulated in a gene expression dataset from a sample that is sensitive to the treatment as compared to a sample that exhibits resistance to the treatment, wherein the identifying generates a treatment-response signature. In some examples, the method includes identifying one or more molecular pathways and/or one or more transcriptional regulatory programs in the network that comprise one or more genes that are (a) relatively upregulated in a gene expression dataset from a sample that has not received the treatment as compared to a sample that is sensitive to the treatment, relatively downregulated in a gene expression dataset from a sample that is sensitive to the treatment as compared to a sample that has not received the treatment and/or to a sample that exhibits resistance to the treatment, and relatively upregulated in a gene expression dataset from a sample that exhibits resistance to the treatment as compared to a sample that is sensitive to the treatment; or (b) relatively downregulated in a gene expression dataset from a sample that has not received the treatment as compared to a sample that is sensitive to the treatment, relatively upregulated in a gene expression dataset from a sample that is sensitive to the treatment as compared to a sample that has not received the treatment and/or to a sample that exhibits resistance to the treatment, and relatively downregulated in a gene expression dataset from a sample that exhibits resistance to the treatment as compared to a sample that is sensitive to the treatment.. In some embodiments, the treatment-response signature identifies a subject that is predicted to be sensitive to the treatment. In some embodiments, the method includes providing the treatment to the subject if the subject is predicted to be sensitive to the treatment.

Also provided are one or more computer-readable media having encoded thereon computer-executable instructions that, when executed, cause a computing system to perform a treatment-response signature identification method, including receiving a gene expression dataset from one or more subjects having a condition or disease (such as a cancer, such as a prostate cancer); or receiving a gene expression dataset from one or more cell cultures representative of the condition or disease (such as a cancer, such as a prostate cancer); identifying one or more transcriptional regulatory programs and one or more molecular pathways that are enriched in the gene expression dataset; determining one or more relationships between the one or more transcriptional regulatory programs and the one or more molecular pathways enriched in the gene expression dataset, wherein the determining generates at least one network for the gene expression dataset; and i identifying one or more molecular pathways and/or one or more transcriptional regulatory programs in the network that comprise one or more genes that are relatively upregulated or downregulated in a gene expression dataset from a sample that has not received the treatment as compared to a sample that is sensitive to the treatment, relatively upregulated or downregulated in a gene expression dataset from a sample that has not received the treatment as compared to a sample that is resistant to the treatment, or relatively upregulated or downregulated in a gene expression dataset from a sample that is sensitive to the treatment as compared to a sample that exhibits resistance to the treatment, wherein the identifying generates a treatment-response signature. In some examples, the method includes identifying one or more molecular pathways and/or one or more transcriptional regulatory programs in the network that comprise one or more genes that are (a) relatively upregulated in a gene expression dataset from a sample that has not received the treatment as compared to a sample that is sensitive to the treatment, relatively downregulated in a gene expression dataset from a sample that is sensitive to the treatment as compared to a sample that has not received the treatment and/or to a sample that exhibits resistance to the treatment, and relatively upregulated in a gene expression dataset from a sample that exhibits resistance to the treatment as compared to a sample that is sensitive to the treatment; or (b) relatively downregulated in a gene expression dataset from a sample that has not received the treatment as compared to a sample that is sensitive to the treatment, relatively upregulated in a gene expression dataset from a sample that is sensitive to the treatment as compared to a sample that has not received the treatment and/or to a sample that exhibits resistance to the treatment, and relatively downregulated in a gene expression dataset from a sample that exhibits resistance to the treatment as compared to a sample that is sensitive to the treatment. In some embodiments, the treatment-response signature identifies a subject that is predicted to be sensitive to the treatment. In some embodiments, the method includes providing the treatment to the subject if the subject is predicted to be sensitive to the treatment.

The foregoing and other features of the disclosure will become more apparent from the following detailed description, which proceeds with reference to the accompanying figures.

### BRIEF DESCRIPTION OF THE DRAWINGS

FIG. 1 is a block diagram of an example system predicting whether a subject having a condition or disease will be sensitive to a treatment or will have or be at risk of developing resistance to the treatment.

FIG. 2 is a flowchart of an example method predicting whether a subject having a condition or disease will be sensitive to a treatment or will have or be at risk of developing resistance to the treatment.

FIG. 3 is a block diagram of an example system generating molecular pathway and transcriptional regulatory program networks.

FIG. 4 is a flowchart of an example method generating molecular pathway and transcriptional regulatory program networks.

FIG. 5 is a block diagram of an example system identifying treatment-response signatures.

FIG. 6 is a flowchart of an example method identifying treatment-response signatures.

FIG. 7 is a block diagram of an example system predicting a treatment response in a subject.

FIG. 8 is a flowchart of an example method predicting a treatment response in a subject.

FIGS. 9A-9D show MYC pathway activity is specific for predicting response to Enzalutamide in CRPC patients. (FIGS. 9A-9B) c-MYC expression in Intact (DMSO treated) and Enzalutamide-resistant (EnzaRes) (FIG. 9A) LNCaP and (FIG. 9B) C42B cells as shown using the qRT-PCR. P-values were estimated using a one-tailed Welch t-test. ** p-value≤0.01. (FIGS. 9C-9D) Kaplan-Meier survival analysis comparing CRPC patients that received (FIG. 9C) Enzalutamide or (FIG. 9D) Abiraterone after sample collection from the Abida et al. cohort with high and normal/low MYC pathway activity levels. Log-rank p-value, adjusted HR (hazard ratio), and CI (confidence interval) are indicated.

FIGS. 10A-10D show reconstruction of a mechanism-centric systems regulatory network for CRPC patients. (FIG. 10A) Schematic representation of the TR-2-PATH workflow. (First row) Single-patient pathway enrichment analysis and single-patient transcriptional regulatory analysis identifies pathway activity vector and transcriptional regulatory activity vector respectively, pairs of which are then subjected to linear regression analysis to reconstruct a mechanism-centric regulatory network. (Second row) In the network, transcriptional regulatory programs are represented as orange nodes and molecular pathways as green nodes. An edge (black arrow) illustrates that a significant relationship was defined between a transcriptional regulatory program and molecular pathway. (FIG. 10B) Distribution of edge weights across the network, as defined by the bootstrap analysis. The x-axis corresponds to the edge weight and the y-axis to its frequency (probability). (FIG. 10C) (Left) t-SNE clustering of molecular pathways (dots), based on the weights of their incoming edges. (Right) Pathways around MYC are shown as a zoom-in and MYC pathway is shown. FIG. 10D: Bootstrap consistency is confirmed by the similarity of significant edge distributions across bootstrap runs. Consistency of bootstrap runs in SU2C East Coast cohort is demonstrated through comparison of the distribution of number of significant edges for each pathway across runs. Leftmost indicates results from the original (whole) SU2C East Coast dataset and remaining indicates results from the bootstrap runs on the same dataset.

FIGS. 11A-11D show network mining I: Identification of upstream transcriptional regulatory programs that affect MYC pathway and are associated with response to Enzalutamide treatment. (FIG. 11A) Schematic representation of the changes in activity levels of molecular pathways and their upstream transcriptional regulatory programs (sub-networks) as they transition from Intact (treated with DMSO) to Enzalutamide-sensitive (EnzaSens) to Enzalutamide-resistant (EnzaRes) phenotypes. TR and pathway activities are up-regulated in phenotypes 1 and 3 and down-regulated in phenotype 2. (FIG. 11B) Identified upstream transcriptional regulatory programs (MYC-centered sub-network) associated with Enzalutamide treatment affecting MYC pathway, depicted across Intact, EnzaSens, and EnzaRes phenotypes. TR and pathway activities are up-regulated in Intact and EnzaRes and down-regulated in EnzaSens. FIGS. 11C-1D show mechanism-centric network mining identifies molecular pathways and TR programs that govern progression to Enzalutamide resistance. (FIG. 11C) GSEA performed on pathway activity levels between a reference signature comparing Enzalutamide-sensitive (EnzaSens, n=4) to Intact (treated with DMSO, n=4) samples and query signature comparing Enzalutamide-resistant (EnzaRes, n=4) and Enzalutamide-sensitive (EnzaSens, n=4) samples (query set was defined as up-regulated pathways at p-value<0.05). GSEA NES (normalized enrichment score) and p-value were estimated using 1,000 pathway permutations in the reference signature. (FIG. 11D) GSEA performed on TR activity levels between a reference signature comparing Enzalutamide-sensitive (EnzaSens, n=4) to Intact (treated with DMSO, n=4) samples and query signature comparing Enzalutamide-resistant (EnzaRes, n=4) and Enzalutamide-sensitive (EnzaSens, n=4) samples (query set was defined as up-regulated TRs at p-value<0.05). NES (normalized enrichment score) and p-value were estimated using 1,000 TR permutations in the reference signature.

FIG. 12 shows VIF analysis identifies multi-collinearity between the transcriptional regulatory programs affecting MYC pathway. Bar plot representation of VIF (variance inflation factor) analysis. Each bar corresponds to VIF value for the indicated transcriptional regulatory program (shown on x-axis).

FIGS. 13A-13C shows Network mining II: NME2 has the largest independent effect on MYC pathway. (FIG. 13A) Schematic representation of the PLS-inspired approach to prioritize TR programs, based on their effect on a molecular pathway of interest. (Left) TR activity vectors are utilized as inputs, which are then regressed on a pathway to identify non-collinear latent variables (pie charts), which include a linear combination of TR programs, based on their effect on the pathway (slices in each pie). (Middle) These latent variables are utilized to build a circle of correlation, which depicts the relationship between each latent variable and each TR and pathway. (Right) Effect scores are defined to group and prioritize TRs, based on their effect on a pathway. (FIG. 13B) A circle of correlation is utilized to determine the degree of closeness between TR programs and the MYC pathway, based on their effect on each latent variable. (FIG. 13C) Grouping and prioritization of the MYC upstream TR programs. Circle sizes correspond to the TR effect scores. NME2 is determined to have the most significant effect on MYC pathway.

FIGS. 14A-14B show activity levels of NME2 transcriptional program and MYC molecular pathway reveal significant changes across Enzalutamide-related conditions. (FIG. 14A) Expression levels of NME2 activated targets (n=368) across Intact (treated with DMSO), Enza-sensitive (EnzaSens), and Enza-resistant (EnzaRes) phenotypes. (FIG. 14B) Expression levels of MYC pathway genes (n=57) across Intact (treated with DMSO), Enza-sensitive (EnzaSens), and Enza-resistant (EnzaRes) phenotypes.

FIGS. 15A-15D show upregulation of AR, CK8, and CD45 identify adenocarcinoma prostate cancer cells in pre- and post-Enzalutamide conditions. UMAP representation of (FIG. 15A) pre- and post-Enza cell populations, (FIG. 15B) AR activity levels, (FIG. 15C) CK8 expression levels, and (FIG. 15D) CD45 expression levels, identifying a group of adenocarcinoma prostate cells (circle).

FIGS. 16A-16E show activity of MYC and NME2 predict poor response to Enzalutamide. (FIGS. 16A-16B) Comparing NME2 TR and MYC pathway activity between adenocarcinoma cells and other cells in neoadjuvant and adjuvant samples of a CRPC patient obtained from He et al. P-value was estimated using a one-tailed Welch t-test. (FIG. 16C) (Left, top) Pearson correlation analysis between NME2 TR and MYC pathway activity in the Abida et al. cohort subjected to adjuvant Enzalutamide. Pearson r and p-value are indicated. (Left, bottom) Patients with high-MYC and high-NME2 and the rest of the patients (others) were identified. (Right) Kaplan-Meier survival analysis, comparing high-MYC and high-NME2 group to the rest of the patients in Abida et al. cohort subjected to adjuvant Enzalutamide. Log-rank p-value, adjusted HR (hazard ratio), and CI (confidence interval) are indicated. (FIG. 16D) (Left, top) Pearson correlation analysis between NME2 TR and MYC pathway activity in SU2C West Coast cohort subjected to Enzalutamide and/or Abiraterone either before or after sample collection. Pearson r and p-value are indicated. (Left, bottom) Patients with high-MYC and high-NME2 and the rest of the patients were identified. (Right) Kaplan-Meier survival analysis, comparing high-MYC and high-NME2 group to the rest of the patients in SU2C West Coast cohort. Log-rank p-value, adjusted HR (hazard ratio), and CI (confidence interval) are indicated. (FIG. 16E) (Left) Kaplan-Meier survival analysis, comparing high-MYC and high-NME2 group to the rest of the patients in Abida et al. cohort subjected to adjuvant Abiraterone. Log-rank p-value, adjusted HR, and CI are indicated. (Right) ROC analysis using NME2 TR and MYC pathway activity levels to compare CRPC patients with poor and favorable Abiraterone response from PROMOTE cohort. AUROC (area under ROC) is indicated.

FIGS. 17A-17D show AR expression and activity are higher in Enzalutamide resistant phenotypes. (FIGS. 17A-17B) AR expression in Intact (treated with DMSO) and Enzalutamide resistant (EnzaRes) (FIG. 17A) LNCaP and (FIG. 17B) C42B cell lines, as shown using qRT-PCR. (FIG. 17C) Comparing AR expression and (FIG. 17D) AR activity in CRPC patients from Abida et al patient cohort with high MYC activities and normal/low MY activities. P-values were estimated using a one-tailed Welch t-test. * p<0.05, *** p≤0.001

FIGS. 18A-18D show comparative analysis to different computational methods demonstrates superiority of TR-2-PATH approach. Comparison of different methods with respect to their ability to predict Enzalutamide resistance. Methods include TR-2-PATH (high-MYC and high-NME2), differential expression analysis between Intact, EnzaSens and EnzaRes phenotypes (Welch t-test p-value<0.05, top 470 genes including NME2 TR targets and MYC pathway genes, top 470 genes excluding NME2 TR targets and MYC pathway genes), top 10 predictions from Random (survival) Forests (RF) method, and top 10 predictions from Support Vector Machine (SVM) method. Comparison among methods was done using: (FIG. 18A) Kaplan-Meier survival analysis (log-rank p-value indicated), (FIG. 18B) Cox modeling (Wald p-value indicated), (FIG. 18C) crude (unadjusted) hazards ratio, and (FIG. 18D) adjusted (for age at diagnosis and Gleason score) hazards ratio. Circles correspond to hazard ratio values and whiskers to their Confidence Intervals, HR=1 is indicated as a vertical line in (FIGS. 18C-18D).

FIGS. 19A-19F show Kaplan-Meier survival analysis stratified by age at diagnosis, age at biopsy, and Gleason score demonstrates independent predictive ability of NME2 and MYC. Kaplan-Meier survival analysis for CRPC patients, that were subjected to Enzalutamide after sample collection, in Abida et al. cohort, stratified based on (FIGS. 19A-19B) median age at diagnosis: ≤57 (FIG. 19A) and >57 (FIG. 19B). (FIGS. 19C-19D) median age at biopsy: ≤66.3 (FIG. 11C) and >66.3 (FIG. 19D) and (FIGS. 19E-19F) Gleason score: Gleason 6 and 7 (FIG. 19E) and Gleason 8 and 9 (FIG. 19F). C-index (corresponding to AUROC) is indicated. Group 1 corresponds to patients with high NME2 transcriptional and high MYC pathway activity levels. The rest of the patients is represented by Group 2.

FIGS. 20A-20F show ability of MYC and NME2 to predict Enzalutamide-response outperforms known markers of PCa progression and treatment response. (FIGS. 20A-20B) Comparison of MYC and NME2 ability to predict response to Enzalutamide in Abida et al. cohort to known markers of PCa aggressiveness, including (FIG. 20A) transcriptomic and (FIG. 20B) genomic markers. (FIGS. 20C-20D) Comparison of MYC and NME2 ability to predict response to Enzalutamide in Abida et al. cohort to known markers of response to ADT and ARSIs including (FIG. 20C) transcriptomic and (FIG. 20D) genomic markers. (FIGS. 20E-20F) Comparison of MYC and NME2 ability to predict response to Enzalutamide in Abida et al. cohort to known markers of Enzalutamide-response, including (FIG. 20E) transcriptomic and (FIG. 20F) genomic markers. Two-tailed Welch t-test was utilized to calculate p-values to estimate the difference in expression levels between high-MYC and high-NME2 group and the rest of the patients for transcriptomic markers in FIGS. 20A, C, and E. Fisher-exact test was utilized to calculate p-values to estimate the difference in the frequency/occurrence of any genomic alterations between high-MYC and high-NME2 group and the rest of the patients for genomic markers in FIGS. 20B, D, and F.

FIGS. 21A-21F show MYC targeting is beneficial for patients in Enzalutamide-resistant conditions. (FIG. 21A) Drug sensitivity curves of Enzalutamide-naïve, or Enzalutamide-resistant (EnzaRes) C42B cells treated with MYCi975. (FIG. 21B) Colony formation assay using Enzalutamide-resistant (EnzaRes) C42B cells in Intact (treated with DMSO), treated with Enzalutamide (10 μM), MYCi975 (2 μM), or a combination of Enzalutamide+MYCi975 (10 μM+2 μM). Cells were grown in the presence of respective drugs. Representative images are shown. P-value was estimated using a one-tailed Welch t-test. (FIG. 21C) Boyden chamber-based in vitro migration assay using Enzalutamide-resistant (EnzaRes) C42B cells in Intact (treated with DMSO), treated with Enzalutamide (10 μM), MYCi975 (2 μM), or a combination of Enzalutamide+MYCi975 (10 μM+2 μM). Bars represent the quantification of Crystal Violet trapped by migrated cells. P-value was estimated using a one-tailed Welch t-test. (FIG. 21D) Expression of NME2 in Intact and Enzalutamide-resistant (EnzaRes) C42B cells, using the qRT-PCR. P-value was estimated using the one-tailed Welch t-test. (FIG. 21E) Two different siRNAs targeting NME2 were used to downregulate NME2 (left panel) and its effect on MYC expression using qRT-PCR is shown (right panel). (FIG. 21F) Boyden chamber-based in vitro migration assay using Enzalutamide-resistance (EnzaRes) C42B cells in Intact (treated with DMSO) or treated with a combination of Enzalutamide+MYCi975 (10 μM+2 μM) with or without knockdown of NME2. Bars represent the quantification of Crystal Violet trapped by migrated cells. * p-value<0.05, ** p-value≤0.01, *** p-value≤0.001

FIGS. 22A and 22B show MYC inhibition reduces viability and colony formation in Enzalutamide resistant conditions. (FIG. 22A) Drug response curves of Enzalutamide naïve LNCaP or Enzalutamide-resistant LNCaP cells treated with Enzalutamide and/or MYC-i975. (FIG. 22B) Colony formation assay using Enzalutamide-resistant LNCaP cells t(LNCaP-Enza-Res) in intact (treated with DMSO), treated with enzalutamide (10 μM), MYC-i975 (2 μM), or a combination of Enzalutamide+MYC-i975 (10 μM+2 μM). Cells were grown in the presence of respective drugs. Bars represent quantification of Crystal Violet trapped by migrated cells. P-value is estimated utilizing one-tailed Welch t-test. * p<0.05, *** p≤0.001.

FIG. 23 is a Western blot showing that inducible knockdown of NME2 in C4-2B cells suppresses MYC expression.

FIG. 24 is a block diagram of an example computing system in which described embodiments can be implemented.

FIG. 25 is a block diagram of an example cloud computing environment that can be used in conjunction with the technologies described herein.

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1 and 2 are forward and reverse primers for cMYC, respectively.

SEQ ID NOs: 3 and 4 are forward and reverse primers for androgen receptor (AR), respectively.

SEQ ID NOs: 5 and 6 are forward and reverse primers for NME2, respectively.

SEQ ID NOs: 7 and 8 are siRNAs targeting NME2.

SEQ ID NO: 9 is a shRNA targeting NME2.

### DETAILED DESCRIPTION

### EXAMPLE 1—OVERVIEW

Resistance to a treatment is a significant problem in disease management, particularly in patients with progressive disease, such as in patients with metastatic cancers. However, identifying patients who are at risk of having or developing resistance to a treatment prior to administration of the treatment remains a major challenge. Resistance to the chemotherapeutic drug enzalutamide is a one such problem in treating and managing castration resistant prostate cancer (CRPC). Transcriptional-Regulation-2-PATHway (TR-2-PATH) was developed herein as a novel framework to reconstruct a de novo, condition- or disease-specific, mechanism-centric regulatory network that connects molecular pathways with their upstream transcriptional regulatory programs The nodes of the constructed network represent biological mechanisms, such as transcriptional regulatory mechanisms or molecular pathways, making it a comprehensive, multi-layer regulatory view of changes in a cancer cell. Furthermore, connections between nodes represent potential direct or indirect regulatory relationships between upstream transcriptional regulatory program nodes and downstream molecular pathway nodes. Such relationships can represent cancer vulnerabilities and constitute candidates for therapeutic targeting.

TR-2-PATH was used herein to generate reconstructed a prostate cancer-specific mechanism-centric regulatory network, which identified the MYC molecular pathway and NME2 (an upstream transcriptional regulatory program of the MYC molecular pathway) as a treatment-response signature for predicting enzalutamide sensitivity or resistance in CRPC subjects. This systems investigation of mechanisms that govern enzalutamide resistance has shed light on rescue therapeutic strategies for CRPC patients. MYC has been a known player a prostate cancer progression, yet its role in the response to enzalutamide has not been investigated. The treatment-response signature identified using the disclosed TR-2-PATH systems and methods was validated in independent clinical cohorts, wherein NME2 and MYC effectively identified patients at risk of resistance to enzalutamide, independent of clinical and molecular co-variates. Furthermore, experimental investigations demonstrated that targeting MYC and its partner, NME2, can re-sensitize cells that had developed enzalutamide resistance, thus and could provide an effective strategy for patients at risk of enzalutamide resistance and/or for patients who have failed enzalutamide treatment.

### EXAMPLE 2—EXAMPLE EXPRESSION DATA

In any of the examples herein, expression data can take a variety of forms. For example, expression data can include level of expression associated with a gene, such as a list of one or more genes or set of genes, in which each gene is associated with a level of expression. In practice, digital expression data or a digital representation of expression data can be used as input to the technologies. In practice, expression data can take the form of a digital or electronic item such as a file, binary object, digital resource, or the like.

Example expression data can include gene or gene expression data, such as a direct or an indirect measure of genes or gene expression. For example, transcriptomic data can be used as a measure of gene expression. In specific, non-limiting examples, genomic data can include nucleic acid-based data, such as mRNA or miRNA data.

Data obtained using various techniques can be used in the methods herein. For example, gene expression can be detected and quantitated using RNA sequencing (RNA-seq), such as single cell RNA-seq (scRNA-seq) (see Stark, et al., Nat Rev Genet. 2019;20, 631-656; Haque, et al., Genome Med. 2017;9(75)). RNA-seq is most frequently used for analyzing differential gene expression between samples. In traditional RNA-seq analyses, the process of analyzing differential gene expression via RNA-seq begins with RNA extraction (such as from a tumor sample, such as a prostate cancer sample), followed by mRNA enrichment or ribosomal RNA depletion. cDNA is then synthesized, and an adaptor-ligated sequencing library is prepared. The library is sequenced to a read depth of, for example, 10-30 million reads per sample on a high-throughput platform (such as an Illumina platform). The sequencing reads (most often in the form of FASTQ files) are computationally aligned and/or assembled to a transcriptome. The reads are most often mapped to a known transcriptome or annotated genome, matching each read to one or more genomic coordinates. This process is often accomplished using alignment tools such as STAR, TopHat, or HISAT, which each rely on a reference genome. If no genome annotation containing known exon boundaries is available (such as if a reference genome annotation is missing or is incomplete), or if reads are to be associated with transcripts rather than genes, aligned reads can be used in a transcriptome assembly step using tools such as StringTie or SOAPdenovo-Trans. Tools such as Sailfish, Kallisto, and Salmon can associate sequencing reads directly with transcripts, without the need for a separate quantification step. Next, reads that have been mapped to transcriptomic or genomic locations are quantified using tools such as RSEM, CuffLinks, MMSeq, or HTSeq, or the alignment-free direct quantification tools Sailfish, Kallisto, or Salmon. Quantification results are often combined into an expression matrix, with one row for each expression feature (gene or transcript) and one column for each sample, with values being read counts or estimated abundances. Samples are then filtered and normalized to account for differences in expression patterns, read depth, and/or technical biases. Significant changes in expression of individual genes and/or transcripts between sample groups are then statistically modeled using one or more of various tools and computational methods.

scRNA-seq enables the systematic identification of cell populations in a tissue. Short sequences or barcodes may be added during library preparation or by direct RNA ligation, before amplification, to mark a sequence read as coming from a specific starting molecule or cell, such as in scRNA-seq experiments. In a scRNA-seq analysis, a tissue sample (such as a prostate tissue sample, such as a prostate cancer tissue sample) is dissociated, single cells are separated, and RNA from each individual cell is converted to cDNA (and can be labelled during reverse transcription) and then amplified (typically using PCR) for sequencing. The synthesized cDNA is used as the input for library preparation. Amplified nucleic acids can also be labelled with barcodes (such as using single-cell combinatorial indexing RNA sequencing or split-pool ligation-based transcriptome sequencing). Tissue dissociation may be accomplished using methods known in the art, such as mechanical disaggregation and/or enzymatic dissociation, such as enzymatic dissociation using collagenase and/or DNase. Similarly, single cells can be separated using known methods, such as flow-cytometry, wherein cells can be flow-sorted directly into micro-plates containing lysis buffer. Individual cells can also be captured in microfluidic chips or loaded into nano-well devices (e.g., by Poisson distribution), isolated, and merged into droplets (containing reagents) via droplet- microfluidic isolation (such as Drop-Seq or InDrop). Isolated single cells are then lysed such that RNA can be released for cDNA synthesis.

Expression data can further include gene or gene expression data from a variety of sources, such as private or publicly accessible databases. For example, databases can include general or specialized databases, such as databases specific for species, taxa, or subject, for example, cancer subjects (such as the Cancer Genome Atlas or the Genomics Data Commons database, portal.gdc.cancer.gov).

Further, in any of the examples herein, expression data can be used with or without additional processing. For example, the methods can include normalization or variance-stabilizing transformation. Other processing is possible, such as centering, standardization, log transformation, rank transformation, and the like.

In any of the examples herein, expression data or its representation can be stored in a database (such as a genomic data database). The database can include expression data with or without additional processing. In particular examples, expression data are stored as a raw or processed RNA-seq data (such as RNA-seq counts, for example, normalized or transformed RNA-seq counts). Precompiled expression data databases may also be used. For example, an application that already has access to a database of pre-computed expression data can take advantage of the technologies without having to compile such a database. Such a database can be available locally, at a server, in the cloud, or the like. In practice, a different storage mechanism than a database can be used (such as a sequence table, index, or the like).

### EXAMPLE 3—EXAMPLE SUBJECTS

As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In any of the examples herein, expression data can include data for a variety of subjects or groups of subjects. In practice, subjects can be single subjects or a part of a group (such as a group with a common feature or characteristic, or a cohort, such as a cohort of subjects having a condition or disease, such as a cohort of subjects that have a cancer). In one example, the subject treated and/or analyzed with the disclosed methods has cancer, such as prostate cancer. In some examples, the subject responds positively to enzalutamide therapy, such as a subject who does not develop resistance to enzalutamide therapy. In other examples, the subject has or is likely to develop resistance to enzalutamide therapy.

In examples, data for subjects or groups can be used for generating a network described herein, and/or for identifying one or more treatment-response signatures as described herein. For example, subjects or groups can include known features or phenotypes, such as for generating the network or the one or more treatment-response signatures, and validation thereof (for example, network generating, treatment-response signature generating, or validation subjects, groups, or cohorts). In specific, non-limiting examples, subjects or groups have a condition or disease, such as a cancer (such as a prostate cancer).

In examples, data for subjects or groups can be used to identify subjects with a feature or phenotype, such as a response to a therapeutic intervention. In practice, subjects or groups can include unknown features or phenotypes (for example, query subjects, groups or cohorts), which can then be identified using one or more treatment-response signatures generated as described herein. For example, subjects or groups can have a condition or disease, such as a cancer (such as a prostate cancer), and a treatment-response signature generated as described herein can be used to identify subjects or groups with a phenotype of interest (such as resistance to or a risk of developing resistance to a therapeutic intervention, such as in a subject with prostate cancer).

In some examples, the subject has a cancer. In some examples, the cancer is prostate, pancreatic, stomach cancer, colon cancer, breast cancer, uterine cancer, bladder cancer, head and neck cancer, kidney cancer, liver cancer, ovarian cancer, rectum cancer, or a hematological malignancy (a “blood” cancer).

### EXAMPLE 4—EXAMPLE SAMPLES

The disclosed methods can include obtaining a biological sample from a subject. A “sample” refers to part of a tissue that is either the entire tissue, or a diseased or healthy portion of the tissue. A sample of biological material obtained from a subject can include cells, proteins, and/or nucleic acid molecules. Biological samples include all clinical samples useful for detection or analysis of disease, such as cancer, in subjects. Appropriate samples include any conventional biological samples, including clinical samples obtained from a human or veterinary subject. Exemplary samples include, without limitation, cancer or tumor samples (such as from surgery, tissue biopsy, tissue sections, or autopsy), cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), or fine-needle aspirates. Samples may be used directly from a subject, or may be processed before analysis (such as concentrated, diluted, purified, such as isolation and/or amplification of nucleic acid molecules in the sample). In a particular example, a sample or biological sample is obtained from a subject having, suspected of having, or at risk of having cancer (such as prostate cancer). In a specific example, the sample is a prostate cancer sample. In one particular example, the sample from the subject is a tissue biopsy sample, such as from a subject that has cancer, such as a prostate cancer. In another example, the sample is a fine needle aspirate. In another specific example, the sample from the subject is a prostate tissue sample. In another specific example, the sample from the subject is a lymph node tissue sample. In some examples, the sample is a primary prostate cancer sample. In other examples, the sample is a metastatic prostate cancer sample (such as a sample from a metastatic tumor that originated from a prostate cancer).

In several embodiments, the biological sample is from a subject suspected of having a cancer, such as prostate cancer, pancreatic cancer, stomach cancer, colon cancer, breast cancer, uterine cancer, bladder cancer, head and neck cancer, kidney cancer, liver cancer, ovarian cancer, or rectum cancer. In some embodiments, the biological sample is a tumor sample or a suspected tumor sample. For example, the sample can be a biopsy sample from a cancer or from a lymph node in a patient that has, or is suspected of having, a cancer, such as a prostate cancer. This information can be used, for example, to determine if certain treatments or treatment methods are appropriate for use in the subject.

Samples obtained from a subject (such as from a subject that has a cancer, such as a prostate cancer, such as prostate tissue samples) can be compared to a control. In some embodiments, the control is a prostate cancer sample obtained from a subject or group of subjects known to have favorably responded to enzalutamide therapy (or not to have responded to enzalutamide therapy). In other embodiments, the control is a standard or reference value based on an average of historical values. In some examples, the reference values are an average expression value for each of a molecule from treatment resistance-related molecules from one or more treatment resistance-related molecular pathways and/or transcriptional regulatory programs in a sample (such as a prostate cancer sample) obtained from a subject or group of subjects known to have favorably responded to the treatment (or not to have responded). In some embodiments, the control is a non-cancer sample (such as a non-cancer sample of the same tissue type as the cancer) obtained from a subject or group of subjects known to not have cancer.

Tissue samples can be obtained from a subject, for example, from cancer patients (such as prostate cancer patients) who have undergone tumor resection as a form of treatment. In some embodiments, cancer samples (such as prostate cancer samples) are obtained by biopsy. Biopsy samples can be fresh, frozen or fixed, such as formalin-fixed and paraffin embedded. Samples can be removed from a patient surgically, by extraction (for example by hypodermic or other types of needles), by microdissection, by laser capture, or by other means.

In some examples, the sample is used to generate a suspension of individual cells, such that nucleic acid molecules can be sequenced for individual cells. In some examples, individual cells are bar coded, such as for sequencing analyses (such as RNA sequencing analyses to measure gene expression). In some examples, proteins and/or nucleic acid molecules (e.g., DNA, RNA, miRNA, mRNA) are isolated or purified from the cancer sample (such as a prostate cancer sample) and non-cancer sample. In some examples, the cancer sample (such as a prostate cancer sample) is used directly, or is concentrated, filtered, or diluted.

In some examples, the sample is a cell or a cell culture, such as a mammalian cell, such as a human cell. In particular examples, the sample is a prostate cancer cell, such as a LNCap cell or a C42B cell. Cell culture samples, including single cell samples, can be prepared using any suitable method.

### EXAMPLE 5—EXAMPLE IMPLEMENTATION IDENTIFYING MOLECULAR PATHWAYS

Any of the examples herein can include determining one or more molecular pathways enriched in a network or treatment-response signature disclosed herein, such as by a pathway enrichment identifier. In practice, one or more molecular pathway signatures can be generated. For example, Example 16 describes molecular pathway and transcriptional regulatory program enrichment associated with prostate cancer.

In practice, molecular pathways enriched in a differential expression signature can be determined in a variety of ways. For example, expression of a gene or a set of genes in a gene expression data set can be compared with genes in molecular pathways, such as included in general or specific molecular pathways databases, for example, HALLMARKS, EcoCyc, KEGG, BioCarta, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like (for example, as described in Garcia-Campos et al., Front. Physiol, 6(383), 2015).

In practice, a variety of processing steps can also be applied. For example, processing can include a quantitative comparison. In examples, a statistical comparison can be used, such as the Kolmogorov-Smirnov statistic, Mann-Whitney test, t-tests (for example, Welch's or Student's t-test), chi-square, Fisher's exact test, binomial, probability, hypergeometric distribution, z-score, permutation analysis, kappa statistics, and the like. Other enrichment analysis tools or algorithms can be used, such as singular, gene set, or modular enrichment analysis. In specific, non-limiting examples, gene set enrichment analysis (GSEA) can be used (such as with differential expression signatures that include genes or gene sets that are ranked based on level of differential expression), for example, ErmineJ, FatiScan, MEGO, PAGE, MetaGF, Go-Mapper, ADGO, or the like (such as described in Huang et al., Nucleic Acids Res, 37(1): 1-13, 2009). In another specific, non-limiting example, a signature of interest (such as a signature defined as a list of genes ranked by their differential expression using a two-sample, two-tailed Welch t-test between treatment resistant and treatment sensitive samples) is used as a reference signature and genes from a specific pathway are used as a query gene set in a GSEA analysis. In an exemplary embodiment of a single-sample analysis, gene expression profiles can be scaled and/or standardized (such as z-scored) on a gene level to allow for comparison of gene ranks across different samples. In such embodiments, a single-sample signature can be defined as a list of genes ranked, such as by z-score, and used as a reference signature in a GSEA analysis. For signature- and/or single sample-based GSEA analysis, normalized enrichment scores (NES) and p-values can be defined using multiple gene permutations, such as 100, 200, 300, 400, 500, 600, 700, 800, 900, 1000, 1500, or 2000 gene permutations. NES values from this analysis can be used as molecular pathway activity values, where positive NES correspond to enrichment of molecular pathway genes in an upregulated part of the signature and negative NES correspond to enrichment of molecular pathway genes in a downregulated part of the signature. In some embodiments, NES produced using Fisher's exact test indicate activity levels of corresponding molecular pathways in a gene expression dataset.

In practice, output pathway signatures can take a variety of forms. For example, molecular pathway signatures can include a list of pathways enriched in a particular phenotype, such as a cohort of subjects having a condition or disease, or a cohort of subjects having a treatment response phenotype. In practice, the list of pathways can include a variety of possible pathways. In examples, possible pathways can include the pathways listed in one or more general or specific pathway databases (for example, EcoCyc, KEGG, BioCarta, RegulonDB, MetaCyc STRINGDB, PANTHER, Gene Ontology, REACTOME, MSigDb, Ingenuity Knowledge Base, NCI PID, WikiPathways, Small Molecule Pathway DB, ConsensusPathDB, Pathway Commons, or the like, such as described in Garcia-Campos et al., Front. Physiol, 6(383), 2015), such as during identification of molecular pathways enriched in one or more samples, such as one or more prostate cancer samples. In examples, possible pathways can include pathways listed in a network (such as a network associated with a disease or condition as disclosed herein, such as a network associated with prostate cancer), such as during use or validation. In other examples, possible pathways can include pathways listed in a signature (such as a treatment response signature disclosed herein, such as a signature indicative of sensitivity of a subject to a treatment, or a signature indicative of resistance of a subject to a treatment).

In examples, enriched pathways can be quantified based on the level of enrichment in networks or signatures. For example, an enrichment score (such as a normalized enrichment score) or a p value can be associated with the enriched pathways in the network or signature output. Other forms are possible; for example, quantified gene expression of the genes in the enriched pathways can be the output.

In examples, pathways can be generated based on absolute valued differential expression signatures or signed differential expression signatures. Thus, pathways can also include absolute valued pathway signatures or signed pathway signatures. Single sample pathway signature output can also be signed or absolute valued.

### EXAMPLE 6—EXAMPLE IMPLEMENTATION IDENTIFYING TRANSCRIPTIONAL REGULATORY PROGRAMS

Any of the examples herein can include determining transcriptional regulatory programs enriched in a network or treatment-response signature disclosed herein, such as by a program enrichment identifier. In practice, one or more transcriptional regulatory program signatures can be generated. For example, Example 16 describes molecular pathway and transcriptional regulatory program enrichment associated with prostate cancer.

In some embodiments, a transcriptional regulatory program can include transcriptional regulators (such as transcriptional regulators, transcription factors, and/or co-factors) and their potential transcriptional targets, connected by the transcriptional regulatory relationships. In practice, transcriptional regulatory programs enriched in a differential expression signature can be determined in a variety of ways. For example, expression of a gene or a set of genes in a gene expression data set can be compared with genes in transcriptional regulatory programs, such as those included in general or specific transcriptional regulatory program collections, such as tissue-specific transcriptional regulatory programs, such as a prostate cancer-specific transcriptional regulatory program, such as that described in Aytes et al. (Cancer Cell, 25:638-651, 2014, incorporated herein by reference in its entirety).

In practice, a variety of processing steps can also be applied. For example, processing can include a quantitative comparison. In examples, a statistical comparison can be used, such as the Kolmogorov-Smirnov statistic, Mann-Whitney test, t-tests (for example, Welch's or Student's t-test), chi-square, Fisher's exact test, binomial, probability, hypergeometric distribution, z-score, permutation analysis, kappa statistics and the like. Other transcriptional regulatory program enrichment analysis tools or algorithms can be used, such as singular, gene set, or modular enrichment analysis. In specific, non-limiting examples, transcriptional regulatory program gene set enrichment analysis (GSEA) can be used (such as with differential expression signatures that include genes or gene sets that are ranked based on level of differential expression), for example, ErmineJ, FatiScan, MEGO, PAGE, MetaGF, Go-Mapper, ADGO, or the like (such as described in Huang et al., Nucleic Acids Res, 37(1): 1-13, 2009). In another specific, non-limiting example, a signature of interest (such as a preliminary treatment-response signature defined as a list of genes ranked by their differential expression using a two-sample, two-tailed Welch t-test between treatment resistant and treatment sensitive samples) is used as a reference signature and genes from a specific pathway are used as a query gene set in a GSEA analysis. In an exemplary embodiment of a single-sample analysis, gene expression profiles can be scaled and/or standardized (such as z-scored) on a gene level to allow for comparison of gene ranks across different samples. In such embodiments, a single-sample signature can be defined as a list of genes ranked, such as by z-score, and used as a reference signature in a GSEA analysis. For signature- and/or single sample-based GSEA analysis, normalized enrichment scores (NES) and p-values can be defined using multiple gene permutations, such as 100, 200, 300, 400, 500, 600, 700, 800, 900, 1000, 1500, or 2000 gene permutations. NES values from this analysis can be used as transcriptional regulatory program activity values, where positive NES correspond to enrichment of transcriptional regulatory program genes in an upregulated part of the signature and negative NES correspond to enrichment of transcriptional regulatory program genes in a downregulated part of the signature.

In a specific, non-limiting example, activity levels of transcriptional regulatory programs can be determined using MAster Regulator INference algorithm (MARINA; Lefebvre et al. Mol Syst Biol, 6:377-377, 2010) (for a signature-based analysis) and Virtual Inference of Protein-activity by Enriched Regulon (VIPER; Alvarez et al. Nat Genet, 48:838-847, 2016) (for single-sample-based analysis) algorithms as described herein. In some embodiments, normalized enrichment scores produced by MARINA and VIPER indicated activity levels of corresponding transcriptional regulatory programs in a gene expression dataset. In some embodiments, normalized enrichment scores produced using Fisher's exact test indicated activity levels of corresponding transcriptional regulatory programs in a gene expression dataset.

In practice, output transcriptional regulatory program signatures can take a variety of forms. For example, transcriptional regulatory program signatures can include a list of transcriptional regulatory programs enriched in a particular phenotype, such as a cohort of subjects having a condition or disease, or a cohort of subjects having a treatment response phenotype. In practice, the list of transcriptional regulatory programs can include a variety of possible programs. In examples, possible transcriptional regulatory programs can include the pathways listed in one or more general or specific transcriptional regulatory program collections, such as tissue-specific transcriptional regulatory programs, such as a prostate cancer-specific transcriptional regulatory program, such as that described in Aytes et al. (Cancer Cell, 25:638-651, 2014, incorporated herein by reference in its entirety). In examples, possible transcriptional regulatory programs can include programs listed in a network (such as a network associated with a disease or condition as disclosed herein, such as a network associated with prostate cancer), such as during use or validation. In other examples, possible transcriptional regulatory programs can include programs listed in a signature (such as a treatment response signature disclosed herein, such as a signature indicative of sensitivity of a subject to a treatment, or a signature indicative of resistance of a subject to a treatment).

In examples, enriched transcriptional regulatory programs can be quantified based on the level of enrichment in networks or signatures. For example, an enrichment score (such as a normalized enrichment score) or a p value can be associated with the enriched transcriptional regulatory programs in the network or signature output. Other forms are possible, for example, quantified gene expression of the genes in the enriched transcriptional regulatory programs can be the output.

In examples, transcriptional regulatory programs can be generated based on absolute valued differential expression signatures or signed differential expression signatures. Thus, transcriptional regulatory programs can also include absolute valued program signatures or signed program signatures. Single sample transcriptional regulatory programs signature output can also be signed or absolute valued.

### EXAMPLE 7—EXAMPLE SYSTEM

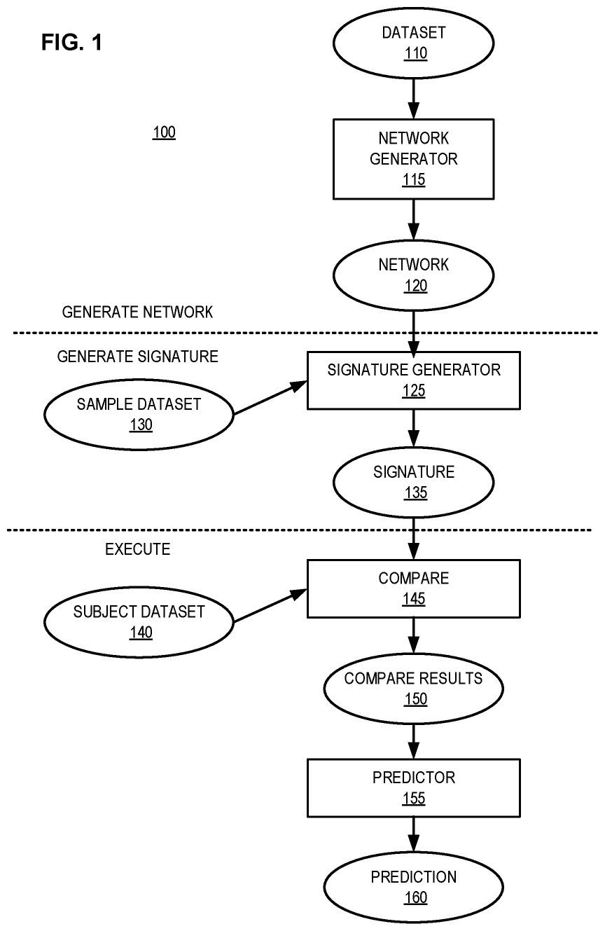

FIG. 1 is a block diagram showing a basic system 100 that can be used to implement predicting a treatment outcome (such as a resistance or a sensitivity to a treatment, such as to enzalutamide) in a subject, such as a subject having cancer, such as prostate cancer, as described herein. The system 100 can be implemented in a computing system as described herein.

In a network generating phase of the example, a network generator 115 receives a dataset 110, such as a gene expression dataset, such as an RNA sequencing dataset, and generates a network 120, such as a network representative of subjects having a condition or disease (such as a prostate cancer). In a signature generating phase of the example, a signature generator 125 receives the network 120 and a dataset for one or more samples 130, such as a gene expression dataset for a sample that has not received a treatment, a gene expression dataset for a sample that is sensitive to the treatment, and/or a gene expression dataset for a sample that exhibits resistance to the treatment. The signature generator 125 generates a signature 135, such as a signature representative of subjects having the condition or disease and a treatment response phenotype (such as resistance or sensitivity to a treatment for the condition or disease).

In an execution phase of the example, a dataset for a subject 140, such as a gene expression dataset for a subject having the condition or disease, is compared 145 to the signature 135 and a predictor 155 receives the results of the comparison 150. The predictor 155 then generates a prediction 160 based on the comparison.

As described herein, in some embodiments, a signature (such as a treatment-response signature) can be compared to a subject dataset to determine whether the subject (such as a subject that has prostate cancer) is predicted to have sensitivity or resistance to a treatment, such as a chemotherapeutic treatment for a cancer, such as a prostate cancer. A subject dataset, such as a gene expression dataset from a subject, can include data from one or more samples from the subject.

In practice, the network generator 115 receives a dataset 110 from one or more samples representing a phenotype of interest, such as one or more samples from one or more subjects that have a condition or disease of interest or samples from one or more cell cultures of one or more cells representative of a condition or disease of interest.

As described herein, the system 100 has been successful in identifying differential molecular pathways and transcriptional regulatory program signatures and in predicting a treatment response in a subject, such as subject that has a cancer, such as a prostate cancer.

In practice, the systems shown herein, such as system 100, can vary in complexity, with additional functionality, more complex components, and the like. For example, there can be additional functionality within the signal generator 115 and/or 130, the comparison function 140, and the predictor function 150. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, academic environment, or the like).

The system 100 and any of the other systems described herein can be implemented in conjunction with any of the hardware components described herein, such as the computing systems described below (e.g., processing units, memory, and the like). In any of the examples herein, the datasets, signatures, pathways, and the like can be stored in one or more computer-readable storage media or computer-readable storage devices. The technologies described herein can be generic to the specifics of operating systems or hardware and can be applied in any variety of environments to take advantage of the described features.

### EXAMPLE 8—EXAMPLE METHOD

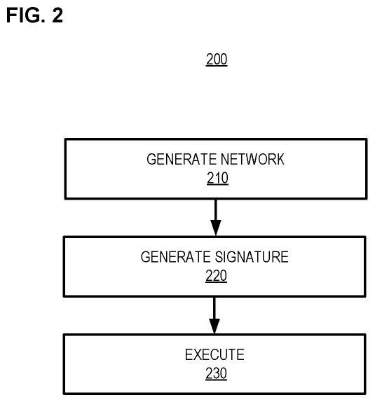

FIG. 2 is a flowchart of an example method 200 predicting a treatment outcome (such as a resistance or a sensitivity to a treatment, such as to enzalutamide) in a subject, such as a subject having cancer, such as prostate cancer, and can be implemented, for example, in the system of that shown in FIG. 1.

In the example, at 210, a network is generated. For example, a network can be generated based on an input dataset from one or more samples having a phenotype of interest.

In the example, at 220, a signature is generated. For example, a signature can be generated based on the network 120 and an old input dataset to predict future outcomes based on a new input dataset. In practice, the system can include one or more signatures as described herein.

At 220, the system executes. For example, the new input dataset can be compared to the signature, providing an output prediction as described herein.

In practice, the network generating, signature generating, and executing acts can be implemented by the same or different parties. For example, one party may perform network generating and signature generating and then provide the signature to be executed in the system (such as compared to a dataset from one or more subjects) by another party. As such, the technologies can be described from a network generating perspective, a signature generating perspective, an execution perspective, or any combination thereof. For example, a network and a signature can be generated as described herein. The signature can then be applied to generate predictions. Alternatively, a network and a signature (e.g., one or both generated earlier) can be received and applied to generate predictions.

The method 200 can incorporate any of the methods or acts by systems described herein to achieve the described technologies.

The method 200 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### EXAMPLE 9—EXAMPLE SYSTEM GENERATING MOLECULAR PATHWAY AND TRANSCRIPTIONAL REGULATORY PROGRAM NETWORKS

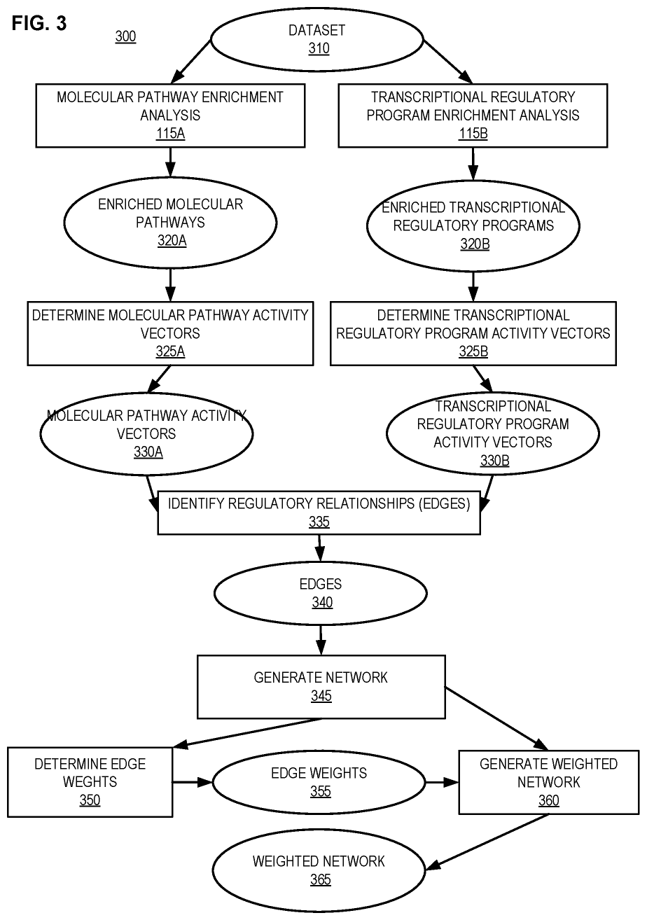

FIG. 3 is a block diagram showing a basic system 300 that can be used to implement generation of molecular pathway and transcriptional regulatory program networks as described herein. The system 300 can be implemented in a computing system as described herein.

In the example, a dataset 310, such as a gene expression dataset, such as an RNA sequencing dataset, is used to perform a molecular pathway enrichment analysis 315A and a transcriptional regulatory program enrichment analysis 315B, and molecular pathways 320A and transcriptional regulatory programs 320B enriched in the dataset are identified. An activity vector is determined for each identified molecular pathway 325A and each identified transcriptional regulatory program 325B, and the resultant molecular pathway activity vectors 330A and transcriptional regulatory program activity vectors 330B are used to identify regulatory relationships (edges) between the molecular pathways and transcriptional regulatory programs enriched in the dataset 335. The edges 340 together generate a network of molecular pathway-transcriptional regulatory program regulatory relationships 345 for the dataset. In some embodiments, a weight is determined for each edge 350 in the network, and the resultant edge weights 355 are incorporated into the network 345 to generate 360 a weighted network 365. Any network of the disclosed systems and methods can be a network 345 or a weighted network 365.

The example shows the system 300 receiving a dataset 310. In practice, the dataset can be any form of dataset, such as any form of gene expression dataset, such as forms of gene expression datasets disclosed herein, from one or more samples representing a phenotype of interest, such as one or more samples from one or more subjects that have a condition or disease of interest or samples from one or more cell cultures of one or more cells representative of a condition or disease of interest. Thus, in some examples, the network 345 or the weighted network 365 generated using the system 300 includes the molecular pathways and transcriptional regulatory programs, and the potential (unweighted or weighted) regulatory relationships between the molecular pathways and transcriptional programs, enriched in the dataset, such as a gene expression dataset from a subject or subjects having a condition or disease (such as a cancer, such as a prostate cancer).

As described herein, the system 300 has been successful in identifying molecular pathway and transcriptional regulatory program networks, such as in a cohort of samples, such as samples from subjects that have a condition or disease, such as a cancer, such as a prostate cancer.

In practice, the systems shown herein, such as system 300, can vary in complexity, with additional functionality, more complex components, and the like. For example, there can be additional functionality within the identify regulatory relationships (edges) function 335, the generate network function 345, and the generate weighted network function 360. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, academic environment, or the like).

The system 300 and any of the other systems described herein can be implemented in conjunction with any of the hardware components described herein, such as the computing systems described below (e.g., processing units, memory, and the like). In any of the examples herein, the datasets, signatures, pathways, and the like can be stored in one or more computer-readable storage media or computer-readable storage devices. The technologies described herein can be generic to the specifics of operating systems or hardware and can be applied in any variety of environments to take advantage of the described features.

### EXAMPLE 10—EXAMPLE METHOD GENERATING MOLECULAR PATHWAY AND TRANSCRIPTIONAL REGULATORY PROGRAM NETWORKS

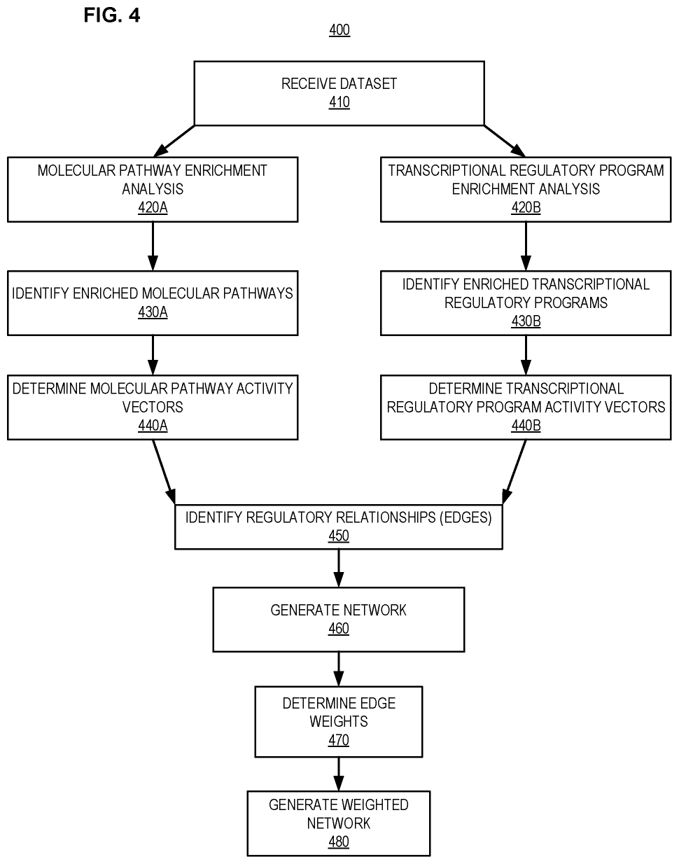

FIG. 4 is a flowchart of an example method 400 generating molecular pathway and transcriptional regulatory program networks and can be implemented, for example, in the system of that shown in FIG. 1 or FIG. 3.

In the example, a molecular pathway enrichment analysis 420A and a transcriptional regulatory program enrichment analysis 420B receive a dataset 410, such as a gene expression dataset, and enriched molecular pathways 430A and enriched transcriptional regulatory programs 430B are identified. A molecular pathway activity vector is identified 440A for each enriched molecular pathway, and a transcriptional regulatory program activity vector is identified 440B for each enriched transcriptional regulatory program. Regulatory relationships (edges) are identified 450 between the enriched molecular pathways and enriched transcriptional regulatory programs, based on the molecular pathway and transcriptional regulatory program activity vectors, and a network is generated 460. In some embodiments, edge weights are determined, and a weighted network is generated. Thus, in this example, networks are generated that describe molecular pathways and/or transcriptional regulatory programs (and interactions between the molecular pathways and/or transcriptional regulatory programs, such as weighted interactions), that are enriched in a condition or disease.

In some embodiments, the dataset is a gene expression dataset (such as an RNA expression dataset) for one or more samples from one or more subjects that have a condition or disease of interest or one or more samples from one or more cell cultures of cells representative of a condition or disease of interest. In some examples, the condition or disease is a cancer, such as a prostate cancer. In specific, non-limiting examples, the dataset is a gene expression dataset from a cohort of subjects, such as the Stand Up to Cancer (SU2C) East Coast cohort (described in Example 16), which includes RNA sequencing datasets of castration-resistant prostate cancer patients obtained from biopsies of metastatic tissues, and profiled on Illumina HiSeq 2500.

The example further shows performing molecular pathway enrichment 420A and transcriptional regulatory program enrichment 420B analyses. Such analyses can be performed using any suitable methods, such as those described herein, such as in Examples 5, 6, and 16, such as gene set enrichment analysis (for molecular pathway enrichment analysis) and MARINA/VIPER analysis (for transcriptional regulatory program analysis). Such analyses can include processing the received dataset, such as converting sequencing data (such as from an RNA sequencing dataset) to FASTQ format, normalizing the data, removing duplicate samples, and the like, prior to performing the enrichment analyses. In some examples, performing a molecular pathway enrichment analysis includes (i) measuring expression of genes of the molecular pathways in a received gene expression dataset; (ii) measuring expression of genes of the molecular pathways in a reference gene expression set; and (iii) comparing the expression of the genes of (i) and (ii), thereby generating a set of molecular pathways enriched in the gene expression dataset. In some examples, performing a transcriptional regulatory program enrichment analysis includes (i) measuring expression of genes of the transcriptional regulatory programs in a received gene expression dataset; (ii) measuring expression of genes of the transcriptional regulatory programs in a reference gene expression set; and (iii) comparing the expression of the genes of (i) and (ii), thereby generating a set of transcriptional regulatory programs enriched in the gene expression dataset. In some examples, the reference gene expression dataset is a list of genes ranked by their differential expression, such as ranked using two-sample two-tailed Welch t-test, between sample sets having at least two phenotypes, such as ranked between samples that are treatment (such as enzalutamide) resistant and samples that are treatment (such as enzalutamide) sensitive.

In determining molecular pathway 440A and transcriptional regulatory program activity vectors 440B, in some embodiments, each molecular pathway vector corresponds to the enrichment scores for the molecular pathway across all samples in the receive dataset 410, and each transcriptional regulatory program vector corresponds to the enrichment score for the transcriptional regulatory program across all samples in the received dataset 410. For example, for n samples and k pathways, if the enrichment score of a molecular pathway i in sample j is NES_{i,j}, then the activity vector for molecular pathway i (P_{i activity}) can be defined as.

$${P}_{i activity} = [ \begin{matrix}{NES}_{i , 1} \\ {NES}_{i , 2} \\ ⋮ \\ {NES}_{i , n}\end{matrix} ]$$

Similarly, for n samples and m transcriptional regulatory programs, if the enrichment score of a transcriptional regulatory program t in a sample j is a_{t,j}, then the activity vector for transcriptional regulatory program t (TR_{t activity}) can be defined as

$${TR}_{t activity} = [ \begin{matrix}{a}_{t , 1} \\ {a}_{t , 2} \\ ⋮ \\ {a}_{t , n}\end{matrix} ]$$

In identifying regulatory relationships (edges) 450 between transcriptional regulatory programs and molecular pathways and generating the network 460, in some embodiments, pairwise comparisons of the transcriptional regulatory program activity vectors and molecular pathway activity vectors are performed, such as using linear regression analysis. In some embodiments, a transcriptional regulatory program activity vector can be used as a predictor variable (independent variable) and a molecular pathway activity vector can be used as a response variable (dependent variable) in the pairwise comparisons, such as the linear regression analyses (such as using the lm function in R). In some examples, multiple hypothesis testing (such as using the p.adjust function in R), such as using false discovery rate (FDR) correction, can be performed to identify transcriptional regulatory programs that have a significant regulatory relationship with a molecular pathway. In some examples, a significant regulatory relationship is a regulatory relationship having a FDR <0.05, <0.01, <0.005, or <0.001. In particular examples, a significant regulatory relationship is a regulatory relationship having a FDR <0.05. Each identified regulatory relationship, such as each significant identified regulatory relationship, is an edge of the generated network. Further, in some examples, a positive beta coefficient (which corresponds to a positive slope for the fitted line between a transcriptional regulatory program activity vector and a molecular pathway activity vector) indicates a positive regulatory relationship by the transcriptional regulatory program on the molecular pathway, and a negative beta coefficient (negative slope) indicates a negative regulatory relationship by the transcriptional regulatory program on the molecular pathway.

In some examples, edge weights are determined 470 for the edges in the network, generating a weighted network 480. In determining an edge weight 470 and generating a weighted network 480, in some embodiments, bootstrap analysis can be used to evaluate robustness and determine a weight for each edge. For example, bootstrapped networks, such as 10-500 bootstrapped networks, can be built using data, such as gene expression profiles, sampled from the input dataset (such as samples from one or more subjects that have a condition or disease, such as a cancer, such as a prostate cancer) with replacement. In some embodiments, 10-100, 100-200, 200-300, 300-400, or 400-500 bootstrapped networks, such as 10, 20, 30, 40, 50, 60, 70, 80, 90, 100, 120, 140, 160, 180, 200, 250, 300, 350, 400, 450, or 500 bootstrapped networks are built. In a specific, non-limiting example, 100 bootstrapped networks are built. In each bootstrapped network, regulatory relationships (edges) are determined as described herein. The bootstrapped networks can be used to assign weights to edges in a network, this generating a weighted network 480. In examples, edge weights are defined as the percent (%) of times an edge identified in the network was also identified across the bootstrapped networks (for example, with FDR p<0.05) while maintaining the same regulatory relationship directionality (e g , maintaining a positive or negative beta coefficient as described herein) between a particular transcriptional regulatory program and a particular molecular pathway, across all bootstrap sampled networks, such as all 100 bootstrap sampled networks. These edge weights can then be added to the network to generate a weighted network.

As described herein, the method 400 has been successful in identifying useful networks (such as unweighted and weighted networks). The method 400 can incorporate any of the methods or acts by systems described herein to achieve the described technologies.

The method 400 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

The illustrated actions can be described from alternative perspectives while still implementing the technologies.

### EXAMPLE 11—EXAMPLE SYSTEM IDENTIFYING TREATMENT RESPONSE SIGNATURES

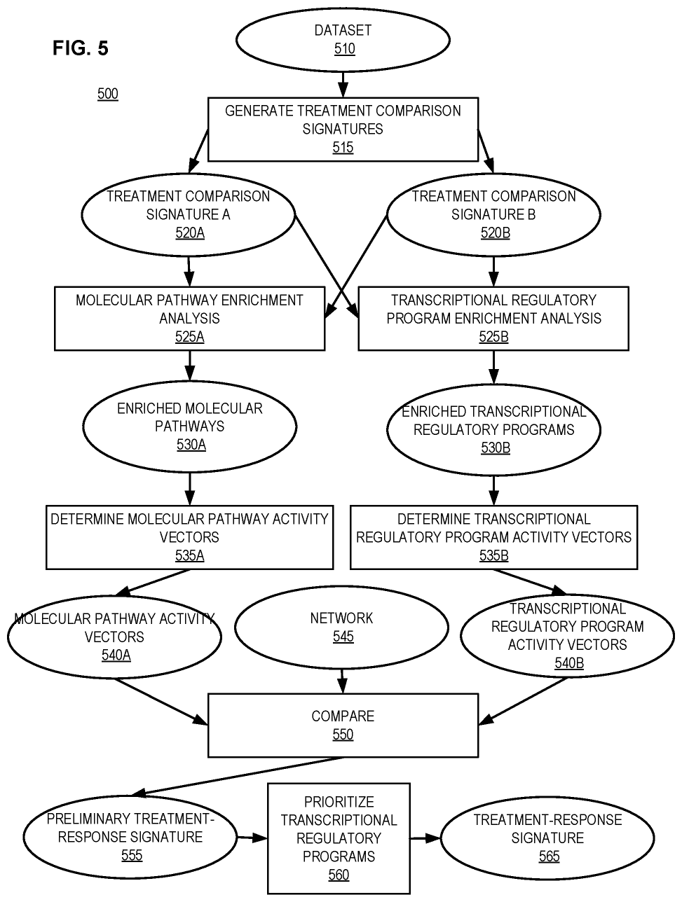

FIG. 5 is a block diagram showing a basic system 500 that can be used to implement identification of one or more treatment response signatures as described herein. The system 500 can be implemented in a computing system as described herein.

In the example, a dataset 510 is used to generate two treatment comparison signatures, treatment comparison signature A 520A and treatment comparison signature B 520B. The treatment comparison signatures each compare at least two treatment phenotypes of interest, such as a signature comparing samples that have not received a treatment to samples that are sensitive to the treatment, and a signature comparing samples that are sensitive to the treatment to samples that have resistance to the treatment; or such as a signature comparing samples that have not received a treatment to samples that are resistant to the treatment, and a signature comparing samples that are sensitive to the treatment to samples that have resistance to the treatment; or such as a signature comparing samples that have not received a treatment to samples that are sensitive to the treatment and a signature comparing samples that have not received a treatment to samples that are resistant to the treatment; a signature comparing samples with different treatment doses, and a signature comparing different treatments. A molecular pathway enrichment analysis 525A and a transcriptional regulatory program enrichment analysis 525B receive the treatment comparison signature A 520A and the treatment comparison signature B 520B separately, and enriched molecular pathways 530A and enriched transcriptional regulatory programs 530B are identified separately for each treatment comparison signature. A molecular pathway activity vector is determined 535A for each enriched molecular pathway in each treatment comparison signature separately, and a transcriptional regulatory program activity vector is identified 535B for each enriched transcriptional regulatory program in each treatment comparison signature separately. A comparison function 550 receives the (1) the molecular pathway activity vectors 540A determined for each enriched molecular pathway in each treatment comparison signature separately, (2) the transcriptional regulatory program activity vectors 540B determined for each enriched transcriptional regulatory program in each treatment comparison signature separately, and (3) a network 545 (such as a weighted network generated as described herein, such as in the system of Example 10, the method of Example 11, or Example 16), and a preliminary treatment-response signature 555 is generated. In some examples, the network is an unweighted network or a weighted network as described herein. Transcriptional regulatory programs of the preliminary treatment response signature are prioritized 560, such as based on level of effect on the molecular pathway of the preliminary treatment response, and a treatment response signature 565 is generated.

Preliminary treatment-response signatures can be a signature or signatures representative of subjects having a treatment response phenotype (such as resistance or sensitivity to a treatment for the condition or disease). Exemplary preliminary treatment-response signatures can comprise a list of molecular pathways and/or transcriptional regulatory programs as described herein that are enriched in a particular treatment response phenotype, such as in samples from subjects having sensitivity or resistance to a treatment. In practice, the list of pathways and programs can include a variety of possible pathways and programs.

In examples, treatment-response signatures can include one or more molecular pathways of the preliminary treatment-response signature, along with a prioritized transcriptional regulatory program associated with each one or more molecular pathway.

As described herein, the system 500 has been successful in identifying treatment-response signatures, such as in one or more samples, such as samples from subjects that have a treatment-response phenotype of interest.

In practice, the systems shown herein, such as system 500, can vary in complexity, with additional functionality, more complex components, and the like. For example, there can be additional functionality within the treatment comparison signature generator 515, the comparison function 550, and the prioritization function 560. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, academic environment, or the like).

The system 500 and any of the other systems described herein can be implemented in conjunction with any of the hardware components described herein, such as the computing systems described below (e.g., processing units, memory, and the like). In any of the examples herein, the datasets, signatures, pathways, and the like can be stored in one or more computer-readable storage media or computer-readable storage devices. The technologies described herein can be generic to the specifics of operating systems or hardware and can be applied in any variety of environments to take advantage of the described features.

### EXAMPLE 12—EXAMPLE METHOD IDENTIFYING TREATMENT RESPONSE SIGNATURES

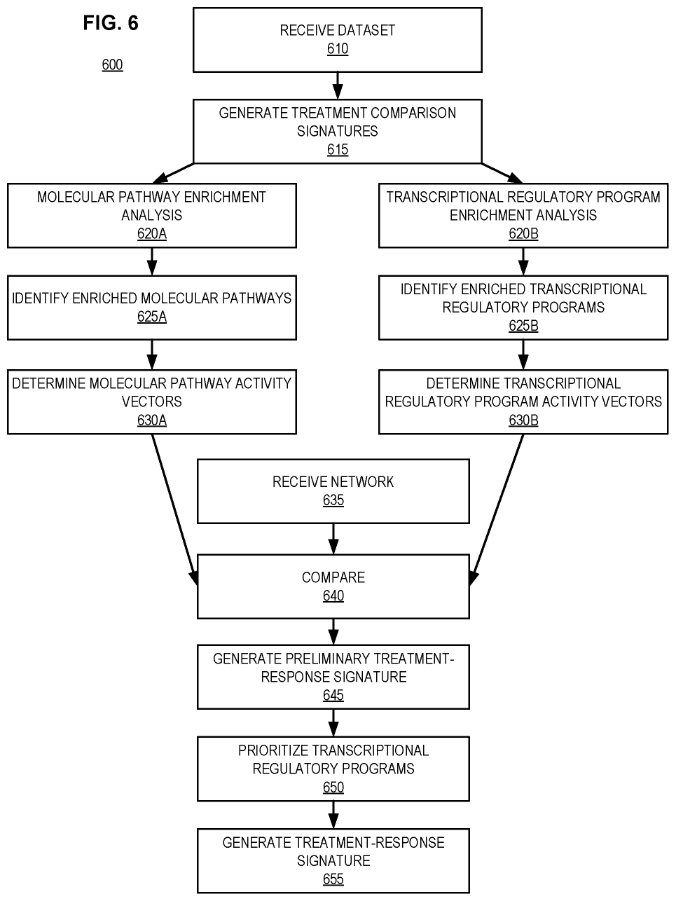

FIG. 6 is a flowchart of an example method 600 identifying differential molecular pathway and transcriptional regulatory program signatures and can be implemented, for example, in the systems of that shown in FIG. 1 and FIG. 5.

In the example, a treatment comparison signature generator 615 receives a dataset 610, for example one or more gene expression datasets, and generates treatment comparison signatures, such as two treatment comparison signatures (e.g., FIG. 5, 520A and 520B). A molecular pathway enrichment analysis 620A and a transcriptional regulatory program enrichment analysis 620B are performed using the treatment comparison signatures (such as the two treatment comparison signatures) separately, and enriched molecular pathways 625A and enriched transcriptional regulatory programs 625B are identified separately for each treatment comparison signature. A molecular pathway activity vector is determined 630A for each enriched molecular pathway in each treatment comparison signature (such as the two treatment comparison signatures) separately, and a transcriptional regulatory program activity vector is identified 630B for each enriched transcriptional regulatory program in each treatment comparison signature (such as the two treatment comparison signatures) separately. A comparison function 640 receives (1) the molecular pathway activity vectors determined for each enriched molecular pathway in each treatment comparison signature separately (2) the transcriptional regulatory program activity vectors determined for each enriched transcriptional regulatory program in each treatment comparison signature separately, and (3) a network 635 (such as a weighted network generated as described herein, such as in the system of Example 9, the method of Example 10, or Example 16), and a preliminary treatment-response signature is generated 645. In some examples, the received network is an unweighted network or a weighted network. Transcriptional regulatory programs of the preliminary treatment-response signature are prioritized 650, and a treatment-response signature is generated 655.

In examples, the dataset includes gene expression datasets for three treatment response phenotypes as follows: (1) a gene expression dataset from one or more samples that have not received the treatment, (2) a gene expression dataset from one or more samples that are sensitive to the treatment, and (3) a gene expression dataset from one or more samples that have resistance to the treatment. In such embodiments, two treatment comparison signatures are generated from the received dataset: (1) a first treatment comparison signature (e.g., of FIG. 5, 520A) comparing samples that have not received a treatment to samples that are sensitive to the treatment, and (2) a second treatment comparison signature (e.g., of FIG. 5, 520B) comparing samples that are sensitive to the treatment to samples that have resistance to the treatment. In some embodiments, treatment comparison signatures can be generated from the dataset using a two-sample, two-tailed Welch t-test, such as using the t.test function in R.

The example shows performing molecular pathway enrichment 620A and transcriptional regulatory program enrichment 620B analyses. Such analyses can be performed using any suitable methods, such as those described herein, such as in Examples 5, 6, 9, 10, and 16, such as gene set enrichment analysis (for molecular pathway enrichment analysis) and MARINA/VIPER analysis (for transcriptional regulatory program analysis). Such analyses can include processing the received dataset, such as converting sequencing data (such as RNA sequencing data) of the dataset to FASTQ format, normalizing the data, removing duplicate samples, and the like, prior to performing the enrichment analyses. In some examples, performing a molecular pathway enrichment analysis includes (i) measuring expression of genes of the molecular pathways in a received gene expression dataset; (ii) measuring expression of genes of the molecular pathways in a reference gene expression set; and (iii) comparing the expression of the genes of (i) and (ii), thereby generating a set of molecular pathways enriched in the gene expression dataset. In some examples, performing a transcriptional regulatory program enrichment analysis includes (i) measuring expression of genes of the transcriptional regulatory programs in a received gene expression dataset; (ii) measuring expression of genes of the transcriptional regulatory programs in a reference gene expression set; and (iii) comparing the expression of the genes of (i) and (ii), thereby generating a set of transcriptional regulatory programs enriched in the gene expression dataset. In some examples, the reference gene expression set is a list of genes ranked by their differential expression, such as ranked using two-sample two-tailed Welch t-test, between sample sets having at least two phenotypes, such as ranked between samples that are treatment (such as enzalutamide) resistant and samples that are treatment (such as enzalutamide) sensitive.

In determining molecular pathway activity vectors 630A and transcriptional regulatory program activity vectors 630B, in some embodiments, each molecular pathway vector corresponds to the enrichment scores for the molecular pathway across a treatment comparison signature, and each transcriptional regulatory program vector corresponds to the enrichment score for the transcriptional regulatory program across a treatment comparison signature. For example, for n samples and k pathways, if the enrichment score of a molecular pathway i in sample j is NES_{i,j}, then the activity vector for molecular pathway i (P_{i activity}) can be defined as

$${P}_{i activity} = [ \begin{matrix}{NES}_{i , 1} \\ {NES}_{i , 2} \\ ⋮ \\ {NES}_{i , n}\end{matrix} ]$$

Similarly, for n samples and m transcriptional regulatory programs, if the enrichment score of a transcriptional regulatory program t in a sample j is a_{t,j}, then the activity vector for transcriptional regulatory program t (TR_{t activity}) can be defined as

$${TR}_{t activity} = [ \begin{matrix}{a}_{t , 1} \\ {a}_{t , 2} \\ ⋮ \\ {a}_{t , n}\end{matrix} ]$$

In some embodiments, in comparing 640 the activity vectors of the first treatment comparison signature, the activity vectors of the second treatment comparison signature, and the activity vectors of the network (such as a described herein, such as in Examples 9, 10, or 16), the molecular pathway activity vectors and transcriptional regulatory program activity vectors for each treatment comparison signature are overlaid with those of the network, to identify parts of the network that exhibit an expression pattern of interest across the two treatment comparison signatures, such as the “up-down-up” behavior described in Example 16. In some examples, to determine if the expression patterns identified in the comparison are statistically significant, molecular pathway-on-molecular pathway and transcriptional regulatory program-on-transcriptional regulatory program GSEA can be performed, wherein molecular pathways of the first treatment comparison signature are compared to molecular pathways of a second treatment comparison signature, and transcriptional regulatory programs of the first treatment comparison signature are compared to transcriptional regulatory programs of the second treatment comparison signature. A preliminary treatment-response signature disclosed herein can comprise parts of the network with significant enrichment in both signatures. For example, molecular pathways and/or transcriptional regulatory programs are identified (1) that are significantly negatively enriched between a gene expression dataset from a sample that has not received a treatment and a gene expression dataset from a sample that is sensitive to the treatment; and (2) that are significantly positively enriched between the gene expression dataset from the sample that is sensitive to the treatment and a gene expression dataset from a sample that is resistant to the treatment. In some examples, parts of the network are considered significantly enriched in both signatures when a GSEA p-vale is less than 0.05, less than 0.01, or less than 0.001, and in opposing directions for the two treatment comparison signatures (i.e., negative significant enrichment in the first treatment comparison signature and positive significant enrichment in the second treatment signature).

In another example, one or more molecular pathways and/or one or more transcriptional regulatory programs in the network can be identified that include one or more genes that are relatively upregulated in a gene expression dataset from a sample that has not received the treatment as compared to a sample that is sensitive to the treatment, relatively downregulated in a gene expression dataset from a sample that is sensitive to the treatment as compared to a sample that has not received the treatment and/or to a sample that exhibits resistance to the treatment, and relatively upregulated in a gene expression dataset from a sample that exhibits resistance to the treatment as compared to a sample that is sensitive to the treatment. In other examples, one or more molecular pathways and/or one or more transcriptional regulatory programs in the network can be identified that include one or more genes that are relatively downregulated in a gene expression dataset from a sample that has not received the treatment as compared to a sample that is sensitive to the treatment, relatively upregulated in a gene expression dataset from a sample that is sensitive to the treatment as compared to a sample that has not received the treatment and/or to a sample that exhibits resistance to the treatment, and relatively downregulated in a gene expression dataset from a sample that exhibits resistance to the treatment as compared to a sample that is sensitive to the treatment. In some examples, the samples are samples from one or more subjects that have a condition or disease, or one or more cell cultures of cells of the condition or disease, such as a cancer, such as a prostate cancer.

In examples, the network used in the compare function 640 includes molecular pathways and/or transcriptional regulatory programs (and interactions between the molecular pathways and/or transcriptional regulatory programs, such as weighted interactions), that are enriched in a condition or disease. In particular examples, the network is as described in Examples 9, 10, or 16.

In some embodiments, prioritizing the transcriptional regulatory programs 650 associated with each molecular pathway of the preliminary treatment-response signature includes identifying the transcriptional regulatory program that has the strongest effect on each pathway. Thus, in prioritizing transcriptional regulatory programs 650 for each one or more molecular pathway of the preliminary treatment-response signature, in some embodiments, (1) at least one non-collinear latent variable is identified using the activity vector for each selected molecular pathway and the activity vector for each selected transcriptional regulatory program, analyzing one pathway at a time; (2) collinear transcriptional regulatory programs are identified and clustered using the at least one non-collinear latent variable; and (3) the transcriptional regulatory program having the closest relationship to each molecular pathway is selected. In such embodiments, the treatment-response signature includes the molecular pathway and the selected transcriptional regulatory program that has the closest relationship to the pathway.

In particular examples, to determine whether any transcriptional regulatory programs associated with a particular pathway are collinear, the activity vectors of the transcriptional regulatory programs can be assessed using variance inflation factor analysis (VIF). VIF performs a multivariable regression analysis setting aside one transcriptional regulatory program at a time. The activity vectors of the remaining transcriptional regulatory programs can be predictor variables (independent variables) and the activity vectors of the transcriptional regulatory program that was set aside as the response variable (dependent variable) to evaluate the percentage of variation that the independent variables could explain about the dependent variable (e.g., as defined by the coefficient of determination, R2). Higher R2 values indicate a higher degree of collinearity. Typically, if VIF>10, co-linearity exists. In specific, non-limiting examples, VIF analysis can be implemented using the vif function from the usdm package in R.

If it is determined that a number of the enriched transcriptional regulatory programs are collinear, at least one non-collinear latent variable can be identified using the activity vector for each selected molecular pathway and the activity vector for each selected transcriptional regulatory program. Collinear transcriptional regulatory programs can be identified and clustered using the at least one non-collinear latent variable. In a specific, non-limiting example, a partial least square (PLS) method can be used to prioritize the transcriptional regulatory programs upstream of each molecular pathway based on the level of effect of each transcriptional regulatory program on its downstream molecular pathway. In such examples, to identify collinear transcriptional regulators, and to group and prioritize the effect of each transcriptional regulator (or group of transcriptional regulators that are collinear), a partial least square regression analysis can be used, that can identify non-collinear latent variables (also referred to as hidden variables). Identification of such latent variables can be used to evaluate indirect relationship between transcriptional regulatory programs and a molecular pathway. In examples, a linear regression analysis can be used to identify latent variables, such as with the activity vector of a transcriptional regulatory program as an independent variable and the activity vector of a pathway as a dependent variable. The effect of each transcriptional regulatory program on a molecular pathway (also referred to herein as a weight) can be indicated by the beta coefficient (slope) of the regression. Using the weights associated with each transcriptional regulatory program's effect on the molecular pathway, and the activity vector of the transcriptional regulatory program, a first latent variable can be determined using the formula:

$$LV 1 = {\sum }_{s = 1}^{m} {TR}_{s activity} * {w}_{s}$$

where m is the number of transcriptional regulatory programs, TR_{s activity} represents activity level of a transcriptional regulatory program across all samples and w represents the weight corresponding to a transcriptional regulatory program. Thus, a latent variable as described herein is a linear combination of transcriptional regulatory programs weighted by their effects on the molecular pathway.

In such examples, to determine the effect of each transcriptional regulatory program on a latent variable (also referred to herein as loadings), a multivariable regression analysis can be performed, where the activity vectors of all transcriptional regulatory programs associated with the molecular pathway are independent variables and the latent variable is the dependent variable. The beta coefficients associated with each transcriptional regulatory program can indicate the effect of each transcriptional regulatory program on the latent variable as adjusted for all other transcriptional regulatory programs

In some examples, to identify a second latent variable, a potential cause-effect relationship between the first latent variable and each transcriptional regulatory program can be determined using linear regression, where the first latent variable is an independent variable and the activity vector of each transcriptional regulatory program is the dependent variable. Residuals of the regression (the difference between an observed value and a predicted value in regression analysis, i.e., a vertical distance between a data point and the regression line) associated with the transcriptional regulatory programs can then be measured. Subsequently, the same analysis can be performed using the first latent variable and the molecular pathway to determine the residuals associated with the molecular pathway. These residuals can be interpreted as the amount of information for each transcriptional regulatory program or molecular pathway that has not been explained by the first latent variable. Using the residuals, the second latent variable can be computed using the same methods as for the first latent variable. This process can be repeated multiple times to determine multiple latent variables (while they can explain a significant amount of information). In particular, non-limiting examples, the PLS method can be implemented using the plsreg1 function of the plsdepot package in R.

In some embodiments, in addition to latent variables, the partial least square regression also identifies the effect of each transcriptional regulatory program (or molecular pathway) on each of the latent variables. In some examples, this is accomplished by performing Pearson correlation between the activity vector of each transcriptional regulatory program or the activity vector of each molecular pathway and each of the latent variables separately. The correlation between an activity vector and the first latent variable (the x variable) and the correlation between an activity vector and the second latent variable as (the y variable) is used to pictorially represent correlations of the transcriptional regulatory programs and the molecular pathway with both latent variables. The pictorial representation (referred to herein as a “circle of correlation”) can then be used identify transcriptional regulatory programs with activity vectors that are co-linear with respect to the effect on a particular molecular pathway of interest. Transcriptional regulatory programs can be grouped based on degree of closeness, which can be determined for each transcriptional regulatory program by calculating the angle (cos⁻¹ θ) between the molecular pathway of interest and the transcriptional regulatory program. A smaller angle between the molecular pathway and the transcriptional regulatory program can indicate a stronger relationship between them.

In some embodiments, because collinear transcriptional regulatory programs may have similar correlation values with the latent variables (and thus a similar degree of closeness), clustering transcriptional regulatory programs by degree of closeness can be used to identify groups of collinear programs. In particular, non-limiting examples, the cos⁻¹ θ can be calculated using the acos function from R. This angle of inclination (in radian) can be converted to degrees, such as using the rad2deg function from the rCAT package in R. To determine the degree of closeness, the angle of inclination of each transcriptional regulatory program can be subtracted from the angle of inclination of the molecular pathway. Hierarchical clustering of the transcriptional regulatory programs based on degree of closeness can then be used to identify and group transcriptional regulatory programs that are collinear with one another in their effects on the molecular pathway. In specific, non-limiting examples, hierarchical clustering can be performed using the hclust function in R.

In some embodiments, selecting the transcriptional regulatory program that has the closest relationship to each molecular pathway of the preliminary treatment-response signature includes (1) calculating the correlation between each transcriptional regulatory program or cluster of collinear regulatory programs and each non-collinear latent variable, and ranking each transcriptional regulatory program or cluster of collinear regulatory programs by level of correlation; (2) calculating degree of closeness between each transcriptional regulatory program or cluster of collinear regulatory programs and each molecular pathway, and ranking each transcriptional regulatory program or cluster of collinear regulatory programs by degree of closeness, and (3) determining the robustness of the relationship between each transcriptional regulatory program or cluster of collinear regulatory programs and each molecular pathway using the calculated edge weights for each relationship, and ranking each transcriptional regulatory program or cluster of collinear regulatory programs by robustness of the relationship. The rankings from steps 1-3 can then be combined to select the transcriptional regulatory program having the closest relationship to each molecular pathway. In such embodiments, the treatment-response signature includes the molecular pathway and the selected transcriptional regulatory program that has the closest relationship to the pathway.

In some examples, in ranking the correlation between each transcriptional regulatory program (or group) and each non-collinear latent variable, the average correlation value can be used for transcriptional regulatory programs that are grouped together. The correlations between each transcriptional regulatory program (or group) and each latent variable separately can be ranked from highest correlation value to lowest correlation value. The transcriptional regulatory program (or group) having the highest correlation with a latent variable can be ranked highest (rank=1) and the transcriptional regulatory program (or cluster) having the lowest correlation with a latent variable can be ranked lowest.

In some examples, in evaluating the degree of closeness between each transcriptional regulatory program and its downstream molecular pathway, the average degree of closeness can be used for collinear transcriptional regulatory programs that are grouped together. The transcriptional regulatory program (or group) that is closest to each molecular pathway can be ranked highest (rank=1) and the transcriptional regulatory program (or group) that is farthest away from the molecular pathway can be ranked lowest.

In some embodiments, to evaluate the robustness of the relationship between each transcriptional regulatory program and its downstream molecular pathway, the weights of the edges from the network (such as the weights calculated using bootstrap analysis) are used. For transcriptional regulatory programs that are collinear and grouped together, the average edge weight can be used. The transcriptional regulatory program (or group) with the highest edge weight can be ranked highest (rank=1) and the transcriptional regulatory program (or group) with the lowest edge weight can be ranked lowest.

In examples, selecting the transcriptional regulatory program that has the closest relationship to each molecular pathway of the preliminary treatment-response signature can include combining the three rankings described above, such as using rank product via geometric mean. In specific, non-limiting examples, geometric mean is incorporated using the geometric.mean function from the psych package in R.

Thus, treatment-response signatures are generated that can include one or more molecular pathways and the transcriptional regulatory program having the closest relationship with each of the one or more molecular pathways, such that expression patterns of the genes of the one or more molecular pathways and the one or more transcriptional regulatory programs are indicative of a treatment response phenotype. For example, the treatment-response signatures described herein can be used to predict if a subject (such as a subject have a cancer, such as a prostate cancer) is sensitive to the treatment or is resistant (or is at risk of becoming resistant) to the treatment. In practice, the list of pathways and/or programs can include a variety of possible pathways and/or programs. As described herein the method 600 has been successful in identifying useful treatment-response signatures.

The method 600 can incorporate any of the methods or acts by systems described herein to achieve the described technologies.

The method 600 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

### EXAMPLE 13—EXAMPLE SYSTEM PREDICTING A TREATMENT RESPONSE IN A SUBJECT

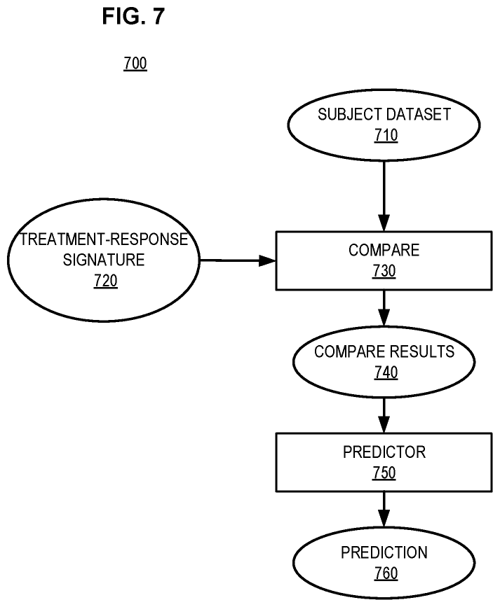

FIG. 7 is a block diagram showing a basic system 700 that can be used to predict a response to a treatment in a subject having a condition or disease as described herein. The system 700 can be implemented in a computing system as described herein.

In the example, a dataset for a subject 710, such as a gene expression dataset for a subject having the condition or disease, is compared 730 to a treatment-response signature 720 as described herein, and a predictor 750 receives the results of the comparison 740. The predictor 750 then generates a prediction 760 based on the comparison.

As described herein, in some embodiments, treatment-response signature can be compared to a subject dataset to determine whether the subject (such as a subject that has a prostate cancer) is predicted to have sensitivity or resistance (or a risk of developing resistance) to a treatment, such as a chemotherapeutic treatment for a cancer, such as a prostate cancer.

As described herein, the system 700 has been successful in predicting a response to a treatment in a subject, such as in a subject that has a condition or disease, such as a cancer, such as a prostate cancer.

In practice, the systems shown herein, such as system 700, can vary in complexity, with additional functionality, more complex components, and the like. For example, there can be additional functionality within the comparison function 730 and the predictor function 760. Additional components can be included to implement security, redundancy, load balancing, report design, and the like.

The described computing systems can be networked via wired or wireless network connections, including the Internet. Alternatively, systems can be connected through an intranet connection (e.g., in a corporate environment, government environment, academic environment, or the like).

The system 700 and any of the other systems described herein can be implemented in conjunction with any of the hardware components described herein, such as the computing systems described below (e.g., processing units, memory, and the like). In any of the examples herein, the datasets, signatures, pathways, and the like can be stored in one or more computer-readable storage media or computer-readable storage devices. The technologies described herein can be generic to the specifics of operating systems or hardware and can be applied in any variety of environments to take advantage of the described features.

### EXAMPLE 14—EXAMPLE METHOD PREDICTING A TREATMENT RESPONSE IN A SUBJECT

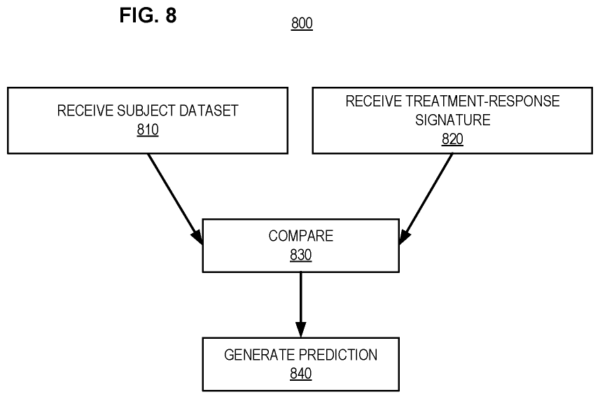

FIG. 8 is a flowchart of an example method 800 predicting a response to a treatment in a subject having a condition or disease and can be implemented, for example, in the systems of that shown in FIG. 1 and FIG. 7.

In the example, a comparison function 830 receives a dataset for a subject 810, such as a gene expression dataset for a subject having the condition or disease, and a treatment-response signature 820, such as a treatment-response signature generated as described herein, such as in Examples 13, 14, or 16. The subject is predicted 840 to be sensitive to the treatment (or resistant to the treatment, or at risk of developing resistance to the treatment) based on the similarity or dissimilarity of, for example, expression levels of genes in the dataset from the subject and the expression levels of the genes of the one or more transcriptional regulatory programs and one or more molecular pathways of the treatment-response signature.

In some examples, the subject dataset is a gene expression dataset, such as an RNA sequencing dataset, such as for one or more samples from the subject. Such a dataset can be used to assess expression of the genes of the one or more molecular pathways and the one or more transcriptional regulatory programs of the treatment-response signature in the subject dataset to determine the extent of similarity or dissimilarity in expression patterns, such as using correlation analysis. In specific, non-limiting examples, the correlation analysis is Pearson correlation, such as implemented using the cor.test function in R.

As described herein the method 800 has been successful in predicting a response to a treatment in a subject.

The method 800 can incorporate any of the methods or acts by systems described herein to achieve the described technologies.

The method 800 and any of the other methods described herein can be performed by computer-executable instructions (e.g., causing a computing system to perform the method) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

### EXAMPLE 15—EXAMPLE IMPLEMENTATION OF RECEIVING EXPRESSION DATA

Any of the examples herein can include receiving a variety of genomic datasets, such as an expression dataset, such as a gene expression dataset (for example, one or more datasets that include one or more datapoints). In practice, expression datasets can include data on genes or sets of genes. For example, a targeted set of genes or a genome-wide set of genes can be included.

In practice, receiving an expression dataset can include expression data for at least one subject (such as a subject with a condition or disease that has not received a treatment, a subject with the condition or disease that has received the treatment and has a known response to the treatment, a subject that does not have the condition or disease, or a subject that has the condition or disease and has an unknown response to the treatment) or at least one group of subjects (such a group of subjects with a common feature or characteristic, or a cohort). In specific, non-limiting examples, receiving an expression dataset can include expression data, such as sequencing data, such as RNA sequencing data, for at least one cohort, such as a cohort with a disease status, such as a cohort that has a condition or disease, such as prostate cancer. In specific, non-limiting examples, the cohort has not received a treatment for the condition or disease.

In further examples, receiving an expression dataset can include receiving expression data, such as sequencing data, such as RNA sequencing data, for at least two cohorts, such as cohorts that have a condition or disease and different treatment statuses (such as two or more cohorts selected from a cohort that has the condition or disease and has not received treatment, a cohort that has received the treatment and is sensitive to the treatment, and a cohort that has received the treatment and is resistant to the treatment). In other specific, non-limiting examples, receiving an expression dataset can include expression data, such as sequencing data, such as RNA sequencing data, for at least three cohorts, such as cohorts that have a condition or disease and different treatment statuses (for example, at least one cohort that has the condition or disease and has not received a treatment, at least one cohort that has received the treatment and is sensitive to the treatment, and at least one cohort that has received the treatment and is resistant to the treatment). For example, FIG. 4 shows receiving 410 a dataset, such as an expression dataset for a cohort (such as a cohort of cancer subjects, such as prostate cancer subjects). In another example, FIG. 6 shows receiving a dataset 610, such as an expression dataset for a first cell or cell culture (such as prostate cancer cells, such as LNCaP or C42B cells, that have not received a treatment), an expression dataset for a second cell or cell culture (such as prostate cancer cells, such as LNCaP or C42B cells, that are sensitive to the treatment), and/or an expression dataset for a third cell or cell culture (such as prostate cancer cells, such as LNCaP or C42B cells, that exhibit resistance to the treatment). In another example, FIG. 6 shows receiving a dataset 610, such as an expression dataset for a first cohort (such as a cohort of cancer subjects, such as prostate cancer subjects, that has not received a treatment), an expression dataset for a second cohort (such as a cohort of cancer subjects, such as prostate cancer subjects, that is sensitive to the treatment), and/or an expression dataset for a third cohort (such as a cohort of cancer subjects, such as prostate cancer subjects, that exhibits resistance to the treatment).

In examples, receiving an expression dataset can include expression data for a subject or subjects with a common feature or characteristic, such as a disease (for example, a cancer, such as a prostate cancer) and/or a treatment response phenotype (for example, a subject or cohort of subjects having prostate cancer and sensitivity to a treatment, or a subject or cohort of subjects having prostate cancer and resistance to the treatment). In specific, non-limiting examples, receiving an expression dataset can include expression data for single subjects or a group of subjects that have prostate cancer.

In practice, receiving an expression dataset can include a variety of processing steps. In examples, processing steps can include normalization, transformation (such as stabilized variance, β value or M value transformation, log transformation, z-score, or rank transformation), redundancy reduction (for example, based on statistical factor, such as a highest coefficient of variation), centering, standardization, logit transformation, bias correction, background correction, and the like.

### EXAMPLE 16—EXAMPLE IMPLEMENTATION OF TRANSCRIPTIONAL-REGULATION-2-PATHway (TR-2-PATH) FOR PREDICTING A TREATMENT RESPONSE IN A SUBJECT

One of the most important oncogenes that has been observed to be overexpressed in almost 60% of CRPC is MYC (Rebello et al., Genes 8:71, 2017). MYC is known to stimulate cell growth and also has been associated with pro-tumorigenic activation in PCa. For example, MYC is known to upregulate EZH2, which is associated with prostate cancer progression. Apart from disease progression, a recent study by Arriaga et al. (Nature Cancer 1:1082-1096, 2020) have demonstrated MYC to be associated with poor response to ARSI in CRPC patients. While studies have shown a tight association of MYC activity with disease progression and response to ARSI, a direct implication of MYC activity in predicting response to Enzalutamide has not been elucidated. Thus, to evaluate if MYC-associated mechanisms could serve as a functional biomarker and therapeutic target in Enzalutamide-resistant conditions, disclosed herein is elucidation of a de novo CRPC-specific mechanism-centric regulatory network, which connects biological pathways with their upstream transcriptional regulatory programs This network was mined with signatures of favorable and poor Enzalutamide response using Partial Least Squares (PLS)-inspired approach. The disclosed analysis nominated MYC pathway and its upstream transcriptional regulatory program NME2 as a mechanism that differentiates favorable and poor response to Enzalutamide.

As disclosed herein, this finding was validated in multiple independent patient cohorts including single cell untreated and Enzalutamide-resistant samples and CRPC patients subjected to adjuvant Enzalutamide treatment. Additionally, the data provided herein confirmed poor predictive ability of MYC and NME2 programs in CRPC patients subjected to adjuvant Abiraterone. Furthermore, the ability to predict patients at risk of Enzalutamide resistance is shown to outperform the predictive ability of markers associated to overall PCa aggressiveness, ARSI resistance, and Enzalutamide resistance. Following identification of NME2 and MYC as a marker of response to Enzalutamide, we further explored the utility of MYC-centric therapeutic targeting by subjecting cells from LNCap and C42B cell lines under Enzalutamide naïve and Enzalutamide resistant conditions to Enzalutamide, and observed that therapeutic targeting of MYC programs reverses Enzalutamide -resistant aggressive phenotype and re-sensitizes LNCaP and C42B Enzalutamide resistant cells to Enzalutamide. We further evaluated the proliferative and metastatic capacity of Enzalutamide resistant cells from C42B cell line using colony formation and migration assay and observed that C42B Enzalutamide resistant cells when subjected to MYC inhibitor, Myc-i975 and Enzalutamide treatment experience significant reduction in both proliferative and metastatic capacity. Finally, apart from direct targeting of MYC, our study also demonstrated that by indirect targeting of MYC via NME2 knockdown in Enzalutamide resistant C42B cells and further treating these cells with Enzalutamide resulted in low metastatic capability, indicating that Enzalutamide resistant cells can recover and become sensitive to Enzalutamide even by indirect targeting of MYC via NME2. Thus, we propose that MYC-associated mechanisms could serve as a biomarker of primary resistance to Enzalutamide aiming to identify patients that are at risk of developing resistance and that should potentially be offered alternative line of treatment. Moreover, our in vitro studies indicate that therapeutic targeting of MYC-associated mechanisms constitutes a valuable primary treatment strategy for these patients and provides a potential secondary rescue therapy for patients that failed Enzalutamide.

### Computational Methods

Datasets utilized: Datasets utilized for network construction, mining, validation, and negative control analysis are summarized in Table 1.

(i) Dataset to associate activity levels of MYC pathway with response to Enzalutamide: To determine if increased activity levels of MYC pathway were associated with Enzalutamide resistance, we utilized Enzalutamide-associated CRPC metastatic samples from Abida et al. (PNAS 116:11328, 2019) cohort (fresh-frozen needle biopsies), profiled on Illumina HiSeq 2500 and downloaded from github.com/cBioPortal/datahub/tree/master/public/prad_su2c_2019. We specifically selected samples that at the time of biopsy (sample collection) were ARSI-naïve (not subjected to any ARSI treatment), treated with Enzalutamide after sample collection, and then followed up for Enzalutamide-associated disease progression (n=22, one sample per patient, as described in Abida et al.). In this sub-group, the mean age at diagnosis was 59 years with a standard deviation of 6.85, the mean age at biopsy was 67.6 years with a standard deviation of 8.3, and the mean prostate-specific antigen (PSA) was 189.4 ng/ml with a standard deviation of 526.18. Metastatic composition of this sub-group included lymph node (n=13), bone (n=6), lung (n=1), other soft tissue (n=1), and liver (n=1) samples. We utilized Enzalutamide-associated disease progression, defined as the time on

Enzalutamide treatment without being subjected to another agent such as taxane, as the clinical end-point (as defined and suggested in Abida et al.).

(ii) Dataset to associate activity levels of MYC pathway with response to Abiraterone: To determine if elevated activity levels of MYC pathway were specifically associated with Enzalutamide (and not Abiraterone) resistance, we utilized Abiraterone-associated metastatic CRPC sample from Abida et al. cohort. We specifically selected samples that at the time of biopsy (sample collection) were ARSI-naïve (as above), treated with Abiraterone after sample collection, and then followed up for Abiraterone-associated disease progression (n=33, one sample per patient) for negative control analysis. The mean age at diagnosis for this patient sub-group was 61.38 years with a standard deviation of 5.94, the mean age at biopsy was 66.73 years with a standard deviation of 7.02, and the mean PSA was 51.4 ng/ml with a standard deviation of 91.05. Metastatic composition of this sub-group included lymph node (n=18), bone (n=11), liver (n=2), and other soft tissue (n=2) samples. We utilized Abiraterone-associated disease progression, defined as the time on Abiraterone treatment, without being subjected to other agents such as taxane, as the clinical end-point (as defined and suggested in Abida et al.).

(iii) Dataset for network reconstruction: To construct a mechanism-centric network, we utilized the Stand Up to Cancer (SU2C) East Coast cohort (Robinson et al., Cell 161:1215-1228, 2015; Abida et al.), profiled on Illumina HiSeq 2500 and downloaded from dbGaP phs000915.v2.p2. This cohort included metastatic CRPC samples, obtained as fresh-frozen needle biopsies. We examined 280 samples available at dbGaP, and to avoid any overlap with treatment-associated analysis in Abida et al. (which we have utilized in part for validation and in part for a negative control) , we removed all SU2C East Coast cohort samples that were present in Abida et al. (n=29). Subsequently, we also removed samples that were duplicated (when the same sample was sequenced by different facilities) and selected one sample per patient to avoid signal duplication for our final network-building. Our final cohort comprised of 153 patients with a mean age at diagnosis of 59.2 years with a standard deviation of 8.38, a mean age at biopsy of 66.1 years with a standard deviation of 8.07, and a mean PSA of 234.5 ng/ml with a standard deviation of 1574.4. Metastatic composition of this cohort included adrenal (n=1), bone (n=39), liver (n=26), lymph node (n=57), other soft tissue (n=19), prostate (n=4), lung (n=2) and unknown origin (n=5) samples. At the time of biopsy (sample collection), patients either were exposed to ARSI (n=67), were ARSI-naïve (n=75), were on treatment (n=4), or their treatment was unknown (n=7).

(iv) Datasets for network mining For network mining (query/interrogation), we utilized LNCaP cell line samples from Kregel et al. (Oncotarget 7:26259-26274, 2016) (n=12), that were profiled with the HumanHT-12 v4 Expression BeadChip and downloaded from GEO GSE78201. These dataset included three phenotypes: (i) LNCaP cells subjected to DMSO (referred to as Intact to indicate that they were not subjected to Enzalutamide treatment) (n=4); (ii) LNCaP cells subjected to Enzalutamide for 48 hours and sensitive to it (referred to as Enzalutamide-sensitive, EnzaSens) (n=4); and (iii) LNCaP cells subjected to Enzalutamide for 6 months and having developed resistance to it (referred as Enzalutamide-resistant, EnzaRes) (n=4).

(v) Datasets for clinical validation: For validation purposes, we utilized (i) He et al. (Nature Medicine 27:426-433, 2021) (ii) Enzalutamide-associated Abida et al., and (iii) SU2C West Coast (Quigley et al., Cell 174:758-769.e759, 2018; Aggarwal et al., Eur. Urol. Focus 2:469-471, 2016) datasets. First, to confirm that upregulation of the NME2 transcriptional regulatory program and MYC pathway characterize Enzalutamide-naïve samples (before patients were exposed to Enzalutamide) from patients that were later exposed to Enzalutamide and eventually failed it, we selected two sequential single-cell samples from the same CRPC patient (01115655) from He et al. cohort. These samples were profiled on Illumina NextSeq 500 and downloaded from singlecell.broadinstitute.org/single_cell/study/SCP1244/transcriptional-mediators-of-treatment-resistance-in-lethal-prostate-cancer. In particular, the first sample was collected before the patient was subjected to Enzalutamide and second sample was collected after the same patient received Enzalutamide and developed resistance to it. Both samples were collected from the lymph-node metastatic site.

Findings from He et al. were confirmed in Enzalutamide-associated Abida et al. cohort. Briefly, we selected a subset of patients that were ARSI-naïve at biopsy, treated with Enzalutamide after sample collection, and subsequently monitored for Enzalutamide-associated disease progression (n=22, as described above).

Further, we validated the predictive ability of NME2 TR and MYC pathway in SU2C West Coast cohort, which comprises of samples from CRPC patients (obtained from fresh frozen image guided core needle biopsies), profiled on Illumina HiSeq 2500 or NextSeq 500 and downloaded from GDC (portal.gdc.cancer.gov/projects/WCDT-MCRPC). The samples in this cohort were subjected to Enzalutamide and/or Abiraterone either before biopsy (sample collection) or after biopsy (sample collection). Subsequently, all patients were monitored for disease progression (n=83, one sample per patient). The mean age for the patients in this cohort was 70.59 years with standard deviation of 8.14. Alongside the patients in this cohort were from various races, including, white (n=70), Asian (n=2), African American (n=5) and unknown (n=6). Metastatic composition of this cohort included bone (n=36), liver (n=7), lymph node (n=31), and unknown (n=9). Further, samples were obtained from patients who were either in M1b stage (i.e., when prostate cancer has spread to bone, n=36) or M1c stage (when prostate cancer has spread to other parts of the body, n=47). We utilized treatment-associated disease progression (defined as an increase in PSA level (minimum 2 ng/mL) that has risen at least twice in an interval of least one week or soft tissue progression (nodal and visceral) based on RECIST v1.1) as the clinical end-point (as defined and suggested in Quigley et al. and Aggarwal et al.).

(vi) Datasets for negative control analysis: To evaluate if the predictive ability of NME2 TR and MYC pathway are indeed Enzalutamide specific, we utilized (i) Abiraterone-associated Abida et al. cohort (as described above); and (ii) PROMOTE (Wang et al., Ann. Oncol. 29:352-360, 2018) cohort, as negative controls. As described above, Abiraterone-associated Abida et al. cohort included ARSI-naïve CRPC samples obtained at biopsy, treated with Abiraterone after sample collection, and subsequently monitored for Abiraterone-associated disease progression (n=33, as described above).

PROMOTE cohort included samples from patients with CRPC profiled on Illumina HiSeq 2500 and downloaded from dbGaP phs001141.v1.p1. These samples were obtained at biopsy from different metastatic sites (n=77, one sample per patient), including bone (n=56), soft tissue (n=2), liver (n=2), prostate bed (n=2), lymph-node (n=14), and lung (n=1) and were ARSI-naïve at the time of sample collection. After sample collection, the patients were subjected to Abiraterone for 12 weeks, and were assessed for Abiraterone-associated disease progression right after that, which was defined based on the score that combined serum PSA level, bone and CT imaging, and symptom assessment at week 12. Patients that developed disease progression at week 12 were classified as non-responders (n=32) and those that did not develop disease progression at week 12 were classified as responders (n=45).

Data download, processing, and normalization: Abida et al. RNA-seq samples profiled on Illumina HiSeq 2500, were downloaded from github.com/cBioPortal/datahub/tree/master/public/prad_su2c_2019 as Fragments Per Kilobase of transcript per Million mapped reads (FPKM). The clinical and treatment data were downloaded from the supplementary material of Abida et al. and from cBioPortal (cbioportal.org/).

SU2C East Coast cohort RNA-seq samples profiled on Illumina HiSeq 2500, were requested and downloaded from dbGaP phs000915.v2.p2 as SRA files using the prefetch command and were converted to FASTQ files utilizing the fastq-dump command from sra toolkit (version 10.8.2). Following this, the FASTQ files were aligned to a reference genome hg19 using STAR aligner with the quantMode option, which generated raw count files. The raw counts were normalized using R DESeq package for further statistical analysis. The clinical data were obtained from the supplementary material of Abida et al. and from cBioPortal.

Kregel et al. LNCaP cell line samples were profiled on HumanHT-12 v4 Expression BeadChip Kit and their quantile-normalized gene expression data were downloaded from GEO GSE78201. The phenotype information was obtained from GEO GSE78201.

He et al. single-cell RNA-seq samples profiled on Illumina Next:Seq 500, were downloaded from singlecell.broadinstitute.org/single_cell/study/SCP1244/transcriptional-mediators-of-treatment-resistance-in-lethal-prostate-cancer, as single-cell Transcripts Per Million (TPM) data matrix. The clinical data were obtained from the main body and supplementary material of He et al.

SU2C West Coast cohort RNA-seq samples profiled on either Illumina HiSeq 2500 or NextSeq 500 were downloaded from GDC (portal.gdc.cancer.gov/projects/WCDT-MCRPC) as BAM files. These BAM files were then converted to FASTQ files utilizing bam2fastq from bedtools. Subsequently, the FASTQ files were aligned to a reference genome hg19 using STAR aligner with the quantMode option, which generated raw count files. The raw counts were normalized using R DESeq for further statistical analysis. The clinical and treatment data were obtained from GDC (portal.gdc.cancer.gov/projects/WCDT-MCRPC).

PROMOTE RNA-seq samples profiled on Illumina HiSeq 2500, were requested and downloaded from dbGaP phs001141.v1.p1 as SRA files using the prefetch command and then converted to FASTQ files using the fastq-dump command from sra toolkit (version 10.8.2). Subsequently, the FASTQ files were aligned to the reference genome hg19 using STAR aligner with the quantMode option to generate raw count files. The raw count files were normalized using R DESeq package. The clinical data were obtained from dbGaP phs001141.v1.p1.

Estimating activity levels of molecular pathways: A list of molecular pathways and their corresponding genes were obtained from Molecular Signatures Database (MSigDB), available from Broad Institute, and included C2 pathway collection (KEGG, BioCarta, and Reactome) and Hallmark (Liberzon et al., Cell Systems 1:417-425, 2015) gene sets. To estimate activity levels of each molecular pathway, we utilized signature-based or single-patient (single-sample) based Gene Set Enrichment Analysis (GSEA), similarly to Epsi et al. (Communications Biology 2:334, 2019) and Rahem et al. (EBioMedicine 61:103047, 2020). For the signature-based GSEA analysis, a signature of interest (e.g., defined as a list of genes ranked by their differential expression using two-tailed Welch t-test between any two phenotypes of interest, such as Enzalutamide-resistant and Enzalutamide-sensitive phenotypes) is used as a reference signature and genes from a specific pathway are used as a query gene set. For single-patient (single-sample) GSEA analysis, gene expression profiles were scaled/standardized (i.e., z-scored) on gene-level so that mean of values for each gene was 0 and the standard deviation was 1, allowing for comparison of gene ranks across different samples. A single-sample signature was defined as a list of genes ranked by their z-scores and utilized as a reference signature in single-sample GSEA analysis (pathway genes were utilized for query, in the same manner as above). For signature-based and single-sample GSEA analysis, Normalized Enrichment Score (NES) and p-values were estimated using 1,000 gene permutations. NESs from this analysis were utilized as pathway activity values, where positive NES corresponds to an enrichment of pathway genes in the over-expressed part of the signature and negative NES corresponds to an enrichment of pathways genes in the under-expressed part of the signature.

Estimating activity levels of Transcriptional Regulatory programs: To estimate the activity levels of transcriptional regulators we utilized MARINa (for a signature-based analysis) and VIPER (for a single-sample-based analysis). Signatures were defined in the same manner as for the pathway enrichment analysis and were utilized as a reference for MARINa/VIPER. Instead of utilizing pathway data, MARINa and VIPER analyses require tissue-specific prostate cancer transcriptional regulatory network (interactome), as reconstructed previously in Aytes et al. (Cancer Cell 25:638-651, 2014). This interactome comprises of transcriptional regulators (TR, transcription factors and co-factors) and their potential transcriptional targets, connected by the transcriptional regulatory relationships. During MARINa/VIPER analysis, these transcriptional targets (for each transcriptional regulator separately) are utilized as a query gene set. We refer to the TR and the set of its corresponding transcriptional targets as a transcriptional regulatory program. Similar to GSEA, NESs/z-scores from MARINa and VIPER analysis were utilized to define activity levels of TRs. MARINa was implemented using msviper function and VIPER was implemented using viper function from R VIPER package in Bioconductor.

TR-2-PATH: reconstruction of a mechanism-centric regulatory network: To identify potential regulatory relationships between molecular pathways and their upstream transcriptional regulatory programs in CRPC patients, we have reconstructed a CRPC-specific mechanism-centric regulatory network, using newly developed TR-2-PATH method. In this network, each node represents a mechanism: a molecular pathway or transcriptional regulatory program. SU2C East Coast cohort (as described above) was first scaled/standardized on the gene level and then subjected to single-sample pathway enrichment analysis (as described above) and single-sample transcriptional regulatory analysis (as described above). We then defined activity vectors for each molecular pathway (where each pathway vector corresponds to the NESs for this pathway across all patients in the SU2C East Coast cohort) and for each TR program (where each TR vector corresponds to the NESs/z-scores for this TR across all patients in SU2C East Coast cohort). Specifically, let us assume that we have n samples. If the activity level of pathway i in sample j is NES_{i,j}, then the activity vector for pathway i, P_{i activity} is defined as.

$${P}_{i activity} = [ \begin{matrix}{NES}_{i , 1} \\ {NES}_{i , 2} \\ ⋮ \\ {NES}_{i , n}\end{matrix} ]$$

Similarly, if the activity level of a TR t in a sample j is a_{t,j}, then the activity vector for TR t, TR_{t activity} is defined as

$${TR}_{t activity} = [ \begin{matrix}{a}_{t , 1} \\ {a}_{t , 2} \\ ⋮ \\ {a}_{t , n}\end{matrix} ]$$

To estimate potential regulatory relationships between transcriptional regulatory programs and molecular pathways, we first performed a pairwise comparison of each TR activity vector and each pathway activity vector using linear regression analysis, where a TR activity vector was used as a predictor variable (independent variable) and pathway activity vector was used as a response variable (dependent variable), as below. For each pathway i and TR t:

P_{i activity}=α+β*TR_{t activity}

The positive Beta (β) coefficient from the linear regression analysis (which corresponds to a positive slope for the fitted line between TR activity vector and pathway activity vector) indicated a positive relationship/association from the TR to the pathway and a negative Beta coefficient (negative slope) indicated a negative relationship/association from the TR to the pathway. Following the regression analysis for all TR-pathway pairs, we subjected it to multiple hypotheses FDR correction, which was performed for each pathway separately. If this relationship showed FDR<0.05, it was added as an edge to the final network. Otherwise, it was discarded. Linear regression analysis was performed using the R lm function and multiple hypotheses testing per pathway was performed using the R p.adjust function.

Bootstrap analysis for the mechanism-centric regulatory network: To evaluate if the edges in the mechanism-centric regulatory network could be “recovered” in the presence of noise (re-sampling), we performed bootstrap analysis. For this, SU2C East Coast cohort gene expression profiles (n=153) were sampled with replacements 100 times. Each sampled/bootstrapped gene expression profile was then used to reconstruct a bootstrapped mechanism-centric regulatory network using the TR-2-PATH method (as above). We then utilized results from these 100 networks to assign weights to each edge, which was defined as the number of times this edge appears (was recovered) across 100 bootstrapped networks (edge frequency). In particular, the edge weights were defined as the percent (%) of times an edge identified in the original network was also identified across the bootstrapped networks while maintaining the same direction of the relationship (positive/negative) between a particular TR program and a particular molecular pathway, across all 100 bootstrapped networks. These edge weights were then added to the original network (making it a weighted mechanism-centric network) and further utilized in the network query step.

The R functions hist and density were utilized to depict weight distributions. To cluster the molecular pathways based on their edge weights, we utilized t-distributed stochastic neighbor embedding clustering (t-SNE), a common dimensionality reduction technique that clustered pathways with similar edge weight patterns as nearby points and pathways with dissimilar edge weight patterns as distal points. t-SNE was implemented using the Rtsne function from R Rtsne package.

Network mining I. Identifying differentially altered sub-networks: To identify parts of the mechanisms-centric network (sub-networks comprising of the molecular pathways and their upstream TR programs) that significantly alter their activity across the response to Enzalutamide, we queried (mined) the mechanism-centric regulatory network using signatures of Enzalutamide-response. In particular, we specifically utilized gene expression profiles from Kregel et al. (as described above), which consists of (i) Intact (DMSO subjected) LNCaP cells (n=4), (ii) Enzalutamide-sensitive (EnzaSens) LNCaP cells (n=4); and (iii) Enzalutamide-resistant (EnzaRes) LNCaP cells (n=4). We hypothesized that if a particular sub-network is up-regulated (positive NES) in the intact state, then becomes down-regulated (negative NES) in the sensitive state, yet “recovers” and again become up-regulated (positive NES) in the resistant state (we call this “up-down-up” behavior), then such sub-network is important in Enzalutamide-resistance and could potentially constitute a functional marker and a therapeutic vulnerability. To identify such sub-networks and establish the significance of this change, we defined two gene expression query signatures (i) signature between intact and sensitive phenotype; and (ii) signature between sensitive and resistant phenotype. These signatures were defined utilizing two-tailed Welch t-test and implemented using the R t.test function.

To identify sub-networks with such “up-down-up” behavior, we evaluated their enrichment in the “intact to sensitive” signature (looking for “up-down” behavior, corresponding to the down-regulation as a result of response to Enzalutamide) and enrichment in the “sensitive to resistant” signature (looking for “down-up” behavior, corresponding to the subsequent up-regulation as a result of resistance to Enzalutamide). To achieve this, we first estimated pathway activity levels and TR activity levels in each signature and overlayed them with our mechanism-centric regulatory network relationships/structure to identify parts of the network that exercise “up-down-up” behavior, as described above. To estimate if such “up-down-up” changes were statistically significant, we performed pathway-on-pathway and TR-on-TR GSEA, where pathways from “intact to sensitive” signature were compared to pathways from “sensitive to resistant” signature (same for the TR programs) Parts of the network with significant negative enrichment in “intact to sensitive” signature and significant positive enrichment in “sensitive to resistant” signature (GSEA p-value <0.001) were utilized for Network mining step II.

### Network Mining II. Prioritization of Upstream Regulatory Programs

Variance Inflation Factor analysis: Sub-networks identified in “Network mining I” include molecular pathways and their potential upstream TR programs. Such TR programs might exercise multi-collinearity in their effect on the pathway and could obstruct further statistical analysis (by making results not interpretable), yet deserve to remain in the analysis (as opposed to simply being eliminated). First, to check for multi-collinearity among TRs, we subjected the activity level of these TRs to Variance Inflation Factor analysis (VIF) in the SU2C East Coast cohort. VIF runs a multivariable regression analysis, iteratively using each TR (activity vector) as a response variable and activity vectors from the rest of the TRs as predictor variables. The percentage of variation that the predictor variables could explain about the response variable is defined by the coefficient of determination, R², where higher R² values indicate a higher degree of multi-collinearity and VIF is defined as 1/(1−R²). Typically, the multi-collinearity is observed if VIF>10. VIF analysis was implemented utilizing the vif function from the R usdm package.

PLS regression analysis: To address TR multi-collinearity, we developed a Partial Least Squares (PLS)-inspired method. To prioritize the effect of TR programs on a specific pathway i, our approach considers TR activity vectors TR_{t activity}, t=1 . . . m (where m is the number of TRs upstream of a specific pathway i) as predictor variables and utilizes a pathway i activity vector P_{i activity} as a response variable. TR activity vectors are then regressed (linear regression) on the pathway vector so that their β coefficients (slopes), indicating the effect of each TR on a pathway i, are denoted as weights wₜ. Next, utilizing the TR activity vectors and weights associated with each TR, first latent variable LV1 is defined as:

$$LV 1 = {\sum }_{t = 1}^{m} {TR}_{t activity} * {w}_{t}$$

Further, the contribution (also referred to as loadings) of each TR on the LV1 is determined through a multivariable regression analysis, where the activity vectors of all the transcriptional regulators TR_{t activity} are utilized as independent variables and the LV1 is utilized as a dependent variable. The β coefficients associated with each TR in this multivariable analysis, indicating the contribution of each TR_{t activity} to LV1, adjusted for the effect of all other TRs, as denoted as loadings. Loadings are most often utilized in social science analyses.

This latent variable LV1 is then “subtracted” from the TR activity vectors and the pathway i activity vector, leaving the residuals to be utilized for defining the next latent variable. In particular, the first latent variable LV1 is utilized as an independent variable to be regressed on the activity vectors of each TR program TR_{t activity} as well as activity of the molecular pathway P_{i activity}, so that the residuals from this analysis explain amount of information that has not been explained by LV1. The residuals are then utilized to define the second latent variable LV2 in the similar fashion. This process is repeated until latent variables can explain a significant amount of information about a pathway i. PLS was implemented utilizing the plsreg1 function from the R plsdepot package.

PLS-inspired circle of correlation analysis: Identified latent variables do not express collinearity or multi-collinearity and are utilized as axes to build a “circle of correlation,” which depicts the association of TR programs and a specific pathway i (defined as arrows on the circle of correlation) to each latent variable. In particular, axes of the circle of correlation depict Pearson correlation r values, defined between latent variables and TR/pathway activity vectors. Each TR and a pathway i are indicated as arrows on the circle of correlation, with x and y coordinates that correspond to the values of Pearson correlation between their vectors and the latent variables.

To identify TRs that affect a specific pathway i as a group, we developed a method that utilized unsupervised hierarchical clustering on the degree of closeness (angle) between TR and pathway arrows so that TRs in high proximity to one another (thus having similar effects on latent variables) are grouped as they express simultaneous effect on the pathway i. In particular, for each TR and pathway arrow we first calculated their angle of inclination (i.e., cos⁻¹ θ). To calculate the cos⁻¹ θ we utilized R acos function. Following this, angle of inclination in radian was converted to a degree using the rad2deg function from the R rCAT package. To determine the degree of closeness, we subtracted the angle of inclination of each TR arrow from angle of inclination of a pathway i arrow. These degrees of closeness for TRs were then subjected to hierarchical clustering, which identified groups of TR programs with similar effects on the pathway i. For hierarchical clustering we utilized the R hclust function.

Prioritizing TR groups: The TR groups/clusters (which also include groups with one TR) are then “prioritized” based on their effect on a pathway i using “effect scores,” which are defined as a combination of (i) degree of closeness between a TR group/cluster and a pathway i on the circle of correlation; (ii) association (Pearson correlation r) between a TR group/cluster and each evaluated latent variable; and (iii) edge weight between a TR group/cluster and a pathway i from the TR-2-PATH mechanism-centric network reconstruction step. For clusters that contained more than one TR, average values for all TRs in that cluster were considered. Each of these categories assigned a “rank” for each cluster and then ranks were combined (using geometric mean) to define the final effect score for each cluster. Geometric mean was implemented utilizing the geometric.mean function from the R psych package.

Validation in independent cohorts and Enzalutamide specificity analysis: For validation and negative control analysis, we utilized He et al., Abida et al., SU2C West Coast and PROMOTE cohorts. Clinical characteristics and data normalization for these cohorts are described above and in Table 1.

In He et al. (single-cell profiles), we reproduced data analysis performed by in the original manuscript. In particular, we applied UMAP dimensionality reduction technique on single-cell Transcripts Per Million (TPM) data for each sample of the selected patient. We then utilized the AR activity and CK8 and CD45 expression on the UMAP projected data to identify adenocarcinoma cell clusters. Next, we estimated NME2 TR activity and MYC pathway activity on a single-cell level, in a manner similar to the single-sample analysis (as described above) and compared their activities between adenocarcinoma cells and the rest of the cells utilizing one-tailed Welch t-test, using the t.test function in R.

In Abida et al., we subjected the cohort samples to a single-sample pathway and single-sample TR analysis to estimate activity levels of MYC pathway and NME2 TR program across all samples. For Enzalutamide-associated subset, we first performed Cook's distance analysis to identify outliers that can influence the regression analysis results (no outliers identified) utilizing R cooks.distance function. Following this, we performed association analyses between activity vectors of NME2 TR and MYC pathway using the R cor.test function. Next to identify patients with high-NME2 TR and high-MYC pathway activities in Enzalutamide-associated subset, we performed hierarchical and kmeans clustering on MYC pathway and NME2 TR activity vectors. For Abiraterone-associated subset, we also performed the Cook's distance analysis (one outlier identified and removed) to identify outliers, followed by identification of patients with high-NME2 TR and high-MYC pathway activities. To identify patients with high-NME2 TR and high-MYC pathway activities, we applied the same thresholds that was estimated in the Enzalutamide-associated subset. Hierarchical clustering was implemented using the R hclust function and kmeans clustering was performed using the R kmeans function and identified two clusters of patients (i) patients with high-NME2 activity and high-MYC pathway activity and (ii) the rest of the patients (e.g., patients with low-NME2 and low-MYC pathway activity; patients with low-NME2 and high-MYC pathway activity; and patients with high-NME2 and low-MYC pathway activity). Further, to evaluate the difference in treatment response between the two identified groups, we utilized Kaplan-Meier survival analysis and Cox proportional hazards model analysis, where treatment-associated disease progression (as described above) was utilized as the clinical end-points, as defined in Abida et al. For Kaplan-Meier survival analysis, we utilized the Surv and the ggsurvplot functions from R survival and survminer packages, respectively. The Cox proportional hazards model analysis was adjusted for age and Gleason score and utilized the coxph function from the R survival package.

In SU2C West Coast cohort, similar to analysis on Abida et al, we subjected the cohort samples to a single-sample pathway and single-sample TR analysis to estimate activity levels of MYC pathway and NME2 TR program across all samples. As above, we first performed Cook's distance analysis to identify outliers (three outliers identified and removed) using R cooks.distance function followed by performing association analyses between activity vectors of NME2 TR and MYC pathway using the R cor.test function. Next, to identify patients with high-NME2 TR and high-MYC pathway activities we utilized hierarchical and kmeans clustering on MYC pathway and NME2 TR activity vectors. Hierarchical clustering was implemented using R hclust function and kmeans clustering was implemented using the R kmeans function and identified two clusters of patients (i) patients with high-NME2 activity and high-MYC pathway activity and (ii) the rest of the patients (e.g., patients with low-NME2 and low-MYC pathway activity; patients with low-NME2 and high-MYC pathway activity; and patients with high-NME2 and low-MYC pathway activity). Further, to evaluate the difference in treatment response between the two identified groups, we utilized Kaplan-Meier survival analyses³⁶ and Cox proportional hazards model analysis, where treatment-associated disease progression (as described earlier) was utilized as the clinical end-points. For Kaplan-Meier survival analysis, we utilized the Surv and the ggsurvplot functions from the R survival and survminer packages respectively. The Cox proportional hazards model analysis was adjusted for race, Mstage, age and metastatic site and utilized the coxph function from the R survival package.

In PROMOTE cohort, we first performed Cook's distance analysis to identify outliers as above (two outliers identified and removed) using R cooks.distance function. Since PROMOTE cohort has binary outcomes (responders vs non-responders), to evaluate the ability of NME2 TR and MYC pathway activities to classify patients based on their binary response to Abiraterone treatment, we performed ROC analysis using a multiplicative logistic regression model, where the product of activity level of the NME2 TR program and activity level of the MYC pathway was utilized as predictor (independent) variable and responder/non-responder classification was utilized as response (dependent) variable. ROC curves were evaluated using area under the curve (AUROC), with AUROC=0.5 being a random classifier. The logistic regression analysis was implemented using the R glm function and ROC analysis was implemented using the roc function from the R pROC package.

Comparison to markers of aggressiveness and therapeutic response: To compare the ability of MYC and NME2 to predict Enzalutamide resistance to the predictive ability of known transcriptomic and genomic markers of aggressiveness and therapeutic response we utilized patients from Enzalutamide-associated Abida et al. cohort (as described above). In particular, comparisons were done in two ways: (i) comparison between high-NME2 and high-MYC pathway patients and the rest of the patients (“others”), as described above using two-tailed Welch t-test (for transcriptomic markers) and Fisher exact test¹⁵¹ (for genomic markers); and (ii) direct independent association with the Enzalutamide-associated disease progression using Cox proportional hazards model. For transcriptomic markers, we utilized their gene expression/normalized counts. For genomic markers, we utilized genomic alterations (obtained from cbioportal), including deep and shallow deletions, gains, and amplifications, as available in cbioportal.

Two-tailed Welch t-test was implemented using the R t.test function, Fisher exact test was implemented using the R fisher.test function, and Cox proportional hazards model analysis was implemented using the coxph function from the R survival package.

Comparative analysis to gene-centric computational methods: To evaluate if TR-2-PATH mechanism-centric predictions (activity levels of NME2 TR and MYC pathway) outperform predictive ability of commonly used gene-centric methods, we compared TR-2-PATH to differential expression analyses, Random (survival) Forests (RF), and Support Vector Machine (SVM) methods all utilized on the Enzalutamide-associated Abida et al. cohort. For differential gene expression analysis, we considered genes that were differentially expressed between the three phenotypes (Intact, EnzaSens, and EnzaRes) in the mining step I and considered genes at (i) Welch t-test p-value<0.05; (ii) top 470 differentially expressed genes (comparable to the total number of target genes and pathway genes used for activity estimation) and not excluding target/pathway genes from NME2 TR and MYC pathway; (iii) top 470 differentially expressed genes, excluding target/pathway genes from NME2 TR and MYC pathway. For RF and SVM analysis, we utilized 470 genes from (iii) to avoid overfitting and then selected top 10 most significant genes/features from the outputs. Final gene list from each of these analyses were utilized to cluster patients using hierarchical and kmeans clustering (as above), and then subjected these groups to Kaplan-Meier survival analysis and Cox proportional hazards model analysis. For Kaplan-Meier survival analysis, we utilized the Surv and the ggsurvplot functions from the R survival and the survminer packages, respectively. Additionally, for Cox proportional hazards model analysis, we utilized the coxph function from the R survival package. For adjusted Cox proportional hazards model analysis, the model was adjusted for age and Gleason score. Random (survival) Forests were constructed utilizing rfsrc function from R randomForestSRC package. The tuning parameters for Random (survival) Forests included (i) the maximum number of trees (“ntrees”), (ii) the number of variables assessed at each split (“mtry”), and (iii) maximum number of samples in the terminal (leaf) nodes (“nodesize”). The optimization of mtry and nodesize variables was performed utilizing tune function from R randomForestSRC package, which determined optimal value for mtry as 100 and nodesize as 5 and iterations of ntrees converged to a stable C-index around 3000, thus 3000 was selected as an optimal value for ntrees. For SVM, we utilized fit function from R rminer package with default parameters.

Data visualization: We utilized the geom_violin and the geom_boxplot function from the ggplot2 in R for data visualization.

TABLE 1
Description of datasets

| | Description and use in this | | | | | Reference |
|---|---|---|---|---|---|---|
| Dataset | study | n | Type of data | Platform | Geo/Link | (PubMed Id) |
| SU2C East | Description: | 153 | RNA | Illumina | dbGaP | PMID: 26000489 |
| Coast | Samples of CRPC patients | | sequencing | HiSeq 2500 | phs000915.v2.p2 | PMID: 31061129 |
| cohort | obtained from biopsies of | | | | | |
| | metastatic tissues. | | | | | |
| | Metastatic sites of biopsy | | | | | |
| | includes, adrenal, bone, liver, | | | | | |
| | lymph node, other soft | | | | | |
| | tissue, prostate, lung and | | | | | |
| | unknown | | | | | |
| | Patients in this cohort are | | | | | |
| | either ARSI exposed or ARSI | | | | | |
| | naïve. | | | | | |
| | Use: | | | | | |
| | Network reconstruction | | | | | |
| Kregel et | Description: | 12 | Microarray | HumanHT-12 | GEO | PMID: 27036029 |
| al | Samples of LNCaP cell line | | gene | v4 | GSE78201 | |
| | from untreated phenotype, | | expression | Expression | | |
| | samples of LNCaP cell line | | | BeadChip Kit | | |
| | from sensitive phenotype | | | | | |
| | and samples of LNCaP cell | | | | | |
| | line from resistant | | | | | |
| | phenotype. | | | | | |
| | Use: | | | | | |
| | Network mining | | | | | |
| He et al | Description: | 2 | Single cell | Illumina | https://singlecell. | PMID: 33664492 |
| | Sequential samples from a | | Transcripts | NextSeq 500 | broadinstitute.org/ | |
| | patient (01115655) that | | Per Millions | | single_cell/study/ | |
| | failed enzalutamide | | (TPM) | | SCP1244/ | |
| | One sample was collected | | | | transcriptional- | |
| | before the patient was | | | | mediators-of- | |
| | subjected to enzalutamide | | | | treatment- | |
| | One sample collected after | | | | resistance- | |
| | the patient developed | | | | in-lethal- | |
| | resistance to Enzalutamide | | | | prostate-cancer | |
| | Use: | | | | | |
| | Clinical Validation | | | | | |
| Abida et al | Description: | 22 | Fragments | Illumina | https://github.com/ | PMID: 31061129 |
| | Samples of patients with | | Per Kilobase | HiSeq 2500 | cBioPortal/datahub/ | |
| | CRPC that were ARSI-naïve at | | of transcript | | tree/master/public/ | |
| | biopsy | | per Million | | prad_su2c_2019 | |
| | After sample collection, | | mapped | | | |
| | patients were treated with | | reads (FPKM) | | | |
| | Enzalutamide | | | | | |
| | Use: | | | | | |
| | Clinical Validation | | | | | |
| SU2C | Description: | 83 | RNA | Illumina | https://portal.gdc. | PMID: 3003370 |
| West | Samples of patients with | | sequencing | HiSeq 2500 | cancer.gov/projects/ | PMID: 28723508 |
| Coast | CRPC that were either | | | NextSeq 500 | WCDT-MCRPC | |
| cohort | treated with ARSI before | | | | | |
| | sample collection of were | | | | | |
| | treated with ARSI after | | | | | |
| | sample collection | | | | | |
| | Use: | | | | | |
| | Clinical Validation | | | | | |
| Abida et al | Description: | 33 | Fragments | Illumina | https://github.com/ | PMID: 31061129 |
| | Samples of patients with | | Per Kilobase | HiSeq 2500 | cBioPortal/datahub/ | |
| | CRPC that were ARSI-naïve at | | of transcript | | tree/master/public/ | |
| | biopsy | | per Million | | prad_su2c_2019 | |
| | After sample collection, | | mapped | | | |
| | patients were treated with | | reads (FPKM) | | | |
| | Abiraterone | | | | | |
| | Use: | | | | | |
| | Negative control | | | | | |
| PROMOTE | Description: | 77 | RNA | Illumina | dbGap | PMID: 29069303 |
| | Samples from patients with | | sequencing | HiSeq 2500 | phs001141.v1.p1 | |
| | CRPPC obtained from | | | | | |
| | biopsies of metastatic tissues | | | | | |
| | After sample collection, | | | | | |
| | patients were subsequently | | | | | |
| | treated with Abiraterone for | | | | | |
| | 12 weeks. | | | | | |
| | Use: | | | | | |
| | Negative control | | | | | |

### Experimental Methods

Generation of Enzalutamide-resistant cell lines: LNCaP (clone FDG) and C42B cells were purchased from ATCC and were grown in RPMI1640 media (GIBCO # 11875093) supplemented with 10% Fetal Bovine Serum (FBS, Corning Cat#35-011-CV) and maintained at 370° C. in and 5% CO2. Enzalutamide powder was purchased from Sellekchem (cat #S1250) and re-suspended in DMSO. Cells were plated in 6 well plates and treated either with DMSO, or with Enzalutamide (20 uM), refreshed every 4 days for up to 3 months until the resistance emerged. RNA from cells was extracted on indicated days using the methods described below.

RNA extraction, cDNA preparation, transcript knockdown, and qRT-PCR analysis: RNA was isolated from cells by the Quick-RNA miniprep kit (Zymogen# R1054) and digested with DNase (provided in the kit). cDNA was synthesized from 1 μg RNA, using an All-in-One 5× RT-master mix (Abm # G592), per the manufacturer's protocol. qRT-PCR was carried out on the StepOne Real-Time PCR system (Applied Biosystems) using gene-specific primers designed with Primer-BLAST and synthesized by IDT Technologies. ON-TARGETplus SMARTpool (cat# L005102-00-0005) was obtained from Dharmacon and was used at 100 nmol/L. Cells were transfected in 6-well plates at a density of 100,000 cells per well using Lipofectamine RNAiMax (Invitrogen #13778075), according to the manufacturer's protocol. RNA was extracted and converted to cDNA as described above. qRT-PCR data were analyzed using the relative quantification method using 18sRNA as an internal reference, and plotted as average fold-change compared with DMSO the non-targeting siRNA (Relative Quantity or RQ). Determination of transcript levels was carried out using Fast SYBR Green Master Mix (Invitrogen), using specific primer sets for c-MYC: c-MYC (F) 5′-CCTGGTGCTCCATGAGGAGAC-3′ (SEQ ID NO: 1); c-MYC (R) 5′- CAGACTCTGACCTTTTGCCAGG-3; (SEQ ID NO: 2).

Evaluating expression of AR: To evaluate the expression of AR in Enzalutamide-naïve and Enzalutamide-resistant conditions, we utilized cells from LNCaP and C42B cell lines under Enzalutamide-naïve and Enzalutamide-resistant conditions (as described above) and determined the expression level of AR under both conditions using qRT-PCR assay (described above). The specific set of primers used for AR includes: AR (F) 5′- TCTTGTCGTCTTCGGAAATGTT-3′ (SEQ ID NO: 3); AR (R) 5′- AAGCCTCTCCTTCCTCCTGTA-3′ (SEQ ID NO: 4).

Evaluating expression of NME2 in Enzalutamide-resistant vs Enzalutamide-nave cells: To evaluate the expression of NME2 in Enzalutamide-naïve and Enzalutamide-resistant conditions, we utilized cells from LNCaP and C42B cell lines under Enzalutamide-naïve and Enzalutamide-resistant conditions (as described above) and determined the expression level of NME2 under both conditions using qRT-PCR assay (as described above). The specific set of primers used for NME2 is: NME2 (F) 5′- AGGATTCCGCCTTGTTGGTCTG-3′ (SEQ ID NO: 5); NME2 (R) 5′- CGGCAAAGAATGGACGGTCCTT-3′ (SEQ ID NO: 6).

Knockdown of NME2: Two different siRNA against NME2 (siNME2#1 AAUAAGAGGUGGACACAAC (SEQ ID NO: 7); siNME2#2 CUGAAGAACACCUGAAGCA (SEQ ID NO: 8)), or non-targeting control (siScram) were obtained from Dharmacon and used at 100 nmol/L.

### Results

### Identification Role of MYC Pathway in Enzalutamide Resistance

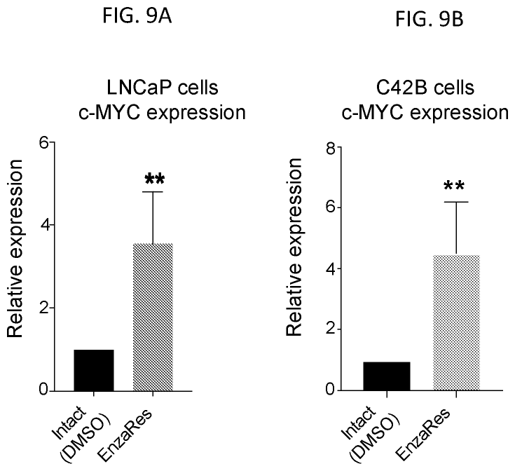

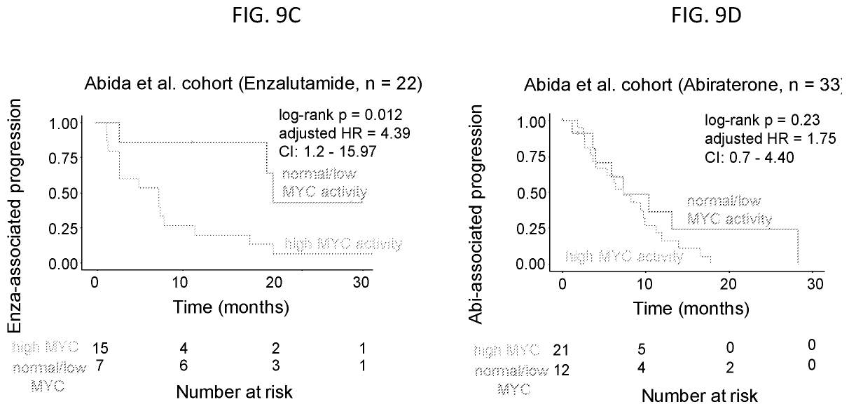

We observed that the levels of MYC are increased in Enzalutamide-resistant conditions (presence of 20 μM Enzalutamide for up to 3 months, EnzaRes) compared to Intact (DMSO) conditions in LNCaP (a cell line derived from prostate cancer metastasis to lymph-node, commonly used to study Enzalutamide resistance) and C42B (LNCaP metastatic CRPC derivative) cell lines (FIGS. 9A-9B, one-tailed Welch t-test p-value=0.002 and p-value=0.0018 for LNCaP and C42B cells, respectively), which were accompanied by increased levels of AR (FIGS. 9A-9B, one-tailed Welch t-test p-value=0.011 and p-value=0.001 for LNCaP and C42B cells, respectively).

To evaluate if this observation could be translated to human patients, we tested if high MYC pathway activity was characteristic of CRPC patients at risk of developing resistance to Enzalutamide. For this, we utilized RNA-seq profiles of CRPC patients from Abida et al., specifically selecting samples from CRPC patients that did not receive any ARSI treatment prior to sample collection (ARS-naïve). We further selected patients that after biopsy (sample collection), were treated with Enzalutamide and monitored for Enzalutamide-associated disease progression (Table 1, n=22). We subjected this patient cohort to single-sample Gene Set Enrichment Analysis (GSEA) to estimate activity levels of MYC pathway in each patient, that were then subjected to unsupervised clustering (see Methods), separating patients with high MYC pathway activity (FIG. 9C, yellow, n=15) and normal/low MYC pathway activity (FIG. 9C, blue, n=7). We then compared Enzalutamide-associated disease progression (which was defined in Abida et al. as the time on Enzalutamide without being subjected to another agent, such as taxane) between these groups using Kaplan-Meier survival analysis and Cox proportional hazards modeling, which demonstrated a significant difference between patients from high MYC and normal/low MYC pathway activity groups (FIG. 9C, log-rank p-value=0.012, adjusted HR (hazard ratio)=4.39, CI (confidence interval): 1.2-15.97), indicating that increased MYC pathway activity is characteristic for patients with a higher risk of resistance to Enzalutamide. The patient group with high MYC pathway activity also demonstrated increased levels of AR expression and activity (see Methods) (FIGS. 9C-9D, one-tailed Welch t-test p-value=0.002 and p-value=0.01, for AR expression and AR activity, respectively) and significant correlation was observed between MYC pathway activity and AR expression/activity levels in the Abida et al. cohort (n=22), as described above (Spearman correlation rho=0.482, p-value=0.024; and Spearman correlation rho=0.484, p-value=0.023, for AR expression and AR activity, respectively).

To further evaluate if this finding was specific to Enzalutamide treatment, we tested if MYC pathway activity could predict treatment response to Abiraterone in CRPC patients. For this, we utilized RNA-seq profiles of CRPC patients from Abida et al. cohort, selecting patients that did not receive any ARSI treatment prior to sample collection and then sub-selecting patients that after biopsy (sample collection) were treated with Abiraterone and monitored for Abiraterone-associated disease progression (which was defined in Abida et al. as the time on Abiraterone without being subjected to another agent, Table 1, n=33). Contrary to the results obtained from Enzalutamide-associated disease progression, we identified no significant difference in Abiraterone-associated disease progression between patients from high MYC and normal/low MYC groups (FIG. 9D, log-rank p-value=0.23, adjusted HR=1.75, CI: 0.7-4.40). Taken together, these analyses demonstrate that increased activity of MYC pathway is indicative of a higher risk of resistance specifically to Enzalutamide and could potentially serve as a marker to stratify patients for their risk of developing resistance to Enzalutamide.

### Defining MYC Upstream Regulatory Programs through Mechanism-Centric Network Analysis

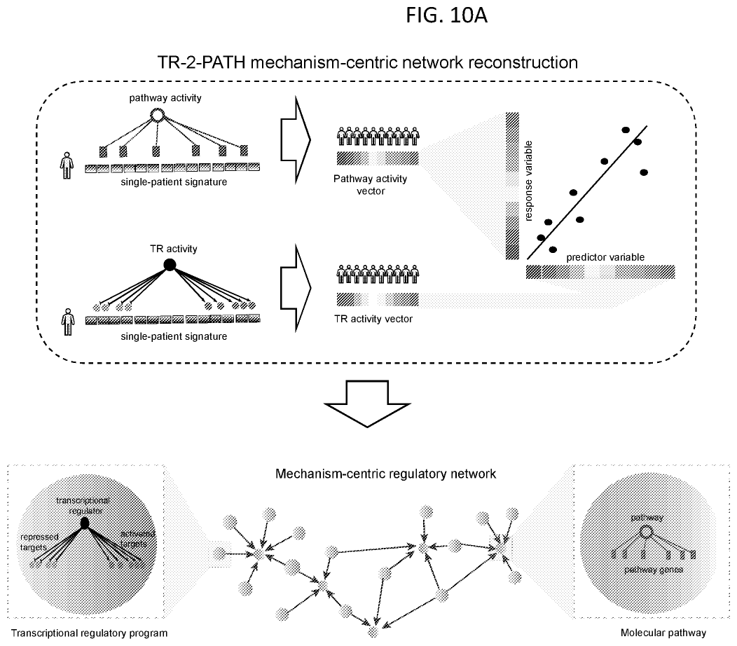

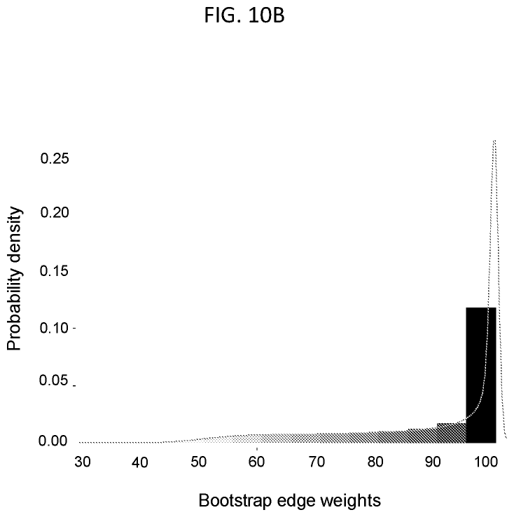

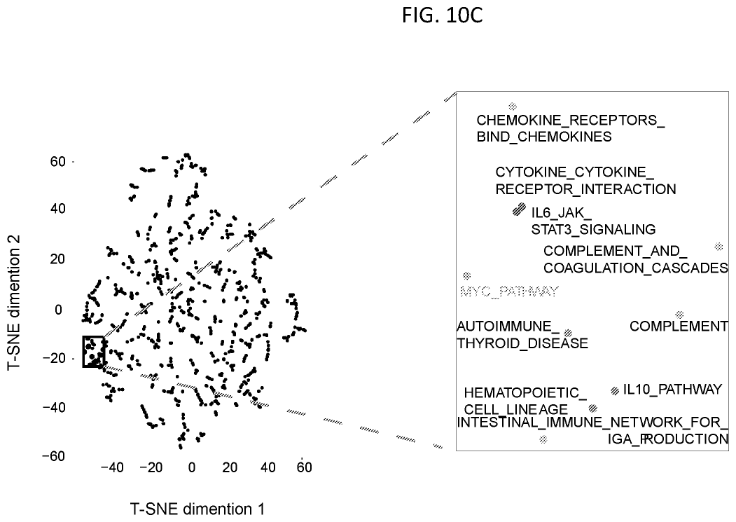

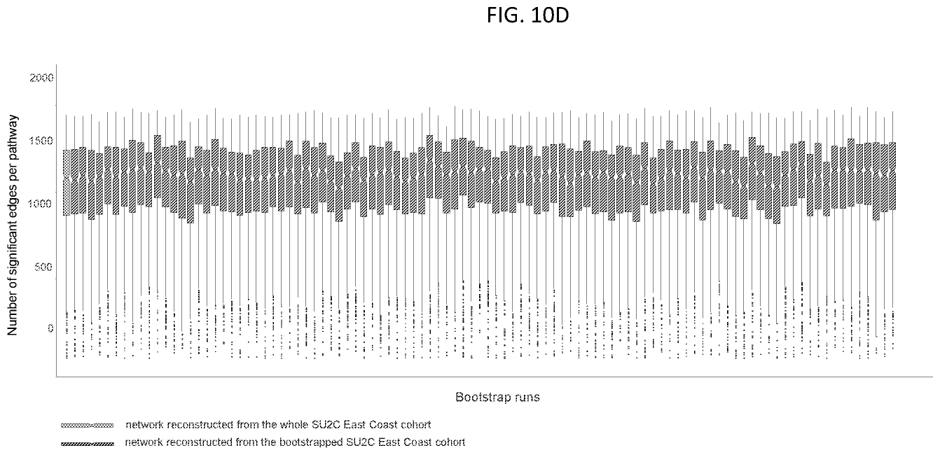

To elucidate MYC regulation implicated in Enzalutamide resistance and define potential additional axes for salvage therapeutics, we investigated transcriptional regulatory mechanisms upstream of MYC pathway that might affect MYC while also governing Enzalutamide resistance. For this, we reconstructed a de novo CRPC-specific mechanism-centric regulatory network, which connects molecular pathways (FIG. 10A) with potential upstream transcriptional regulatory (TR) programs (FIG. 10A), through “TR-2-PATH” method. Nodes in this mechanism-centric network do not correspond to individual genes or alterations, but rather represent mechanisms: such as transcriptional regulatory programs or molecular pathways (FIG. 10A). For TR-2-PATH network reconstruction, we utilized RNA-seq profiles from CRPC patients in the Stand Up to Cancer (SU2C) East Coast cohort, excluding repeated samples and samples from Abida et al. (Table 1, n=153). The selected SU2C East Coast cohort was well-suited for CRPC mechanism-centric network reconstruction as it (i) constitutes the largest-to-date cohort of CRPC patients, essential for statistical learning/inference; (ii) is characterized by wide-ranged age (59.2±8.38 years) and prostate specific antigen (PSA) levels (234.5±1574.4 ng/ml); (iii) includes different metastatic sites, including bone (n=39), liver (n=26), lymph-node (n=57), prostate (n=4), lung (n=2), adrenal (n=1), other soft tissue (n=19), etc.; and (iv) represents different stages of therapeutic intervention, including samples from patients previously exposed to ARSIs (including Enzalutamide and Abiraterone, n=67), ARSI-naïve at the time of sample collection (n=75), currently on treatment (n=4), etc.; all together capturing a wide range of clinical variables necessary for accurate statistical inference.

To evaluate relationships between molecular pathways and their upstream transcriptional regulatory programs, we first needed to estimate pathway activity levels and transcriptional regulator activity levels in each sample in the SU2C East Coast cohort (FIG. 10A). To estimate pathway activity levels, we performed single-sample GSEA on the scaled SU2C East Coast cohort, so that each sample (n=153) was used as a reference signature and each molecular pathway (n=883, from KEGG³⁸, BioCarta, Reactome, and Hallmark collections) was as a query. To estimate activity of TRs in the SU2C East Coast cohort, we performed VIPER analysis on the scaled SU2C East Coast cohort (as above) utilizing each sample (n=153) as a reference and transcriptional regulatory programs (n=2,678) from a prostate cancer specific interactome as a query (see Methods). For each molecular pathway, its activity level (defined as Normalized Enrichment Scores, NES) across all patients in the cohort then defined a “pathway activity vector” (FIG. 10A). Similarly, for each transcriptional regulatory program, its activity level across all patients in the cohort defined a “TR activity vector” (FIG. 10A, bottom). All pairs of TR-pathway activity vectors were then subjected to linear regression analysis (see Methods), where “TR activity vector” was utilized as a predictor (independent) variable and “pathway activity vector” was used as a response (dependent) variable, with an objective to identify TR programs whose changes could potentially explain changes in the activity of molecular pathways in CRPC-specific manner. Significant relationships, corrected for multiple hypotheses testing (see Methods), between TRs and pathways (both positive and negative) were then considered for network reconstruction (FIG. 10A).

To ensure that the network is robust to experimental and sampling noise, we enhanced our network reconstruction with bootstrap analysis. For this, patients from the SU2C East Coast cohort (n=153) were sampled with replacement (see Methods, k=100) and each bootstrap was subjected to TR-2-PATH network reconstruction. A comparison of edge distributions across the 100 bootstrapped networks showed their similarity (FIG. 10E), indicating the method's overall reproducibility. A total of 100 bootstrapped networks were then used to assign “weight” to each edge in the network reconstructed from the whole dataset (n=153), reflecting the number of times an edge appears (and thus could be recovered) across the bootstrapped networks (FIG. 10B). Unsupervised t-distributed stochastic neighbor embedding clustering (t-SNE) was utilized to cluster molecular pathways based on weights of their incoming edges, demonstrating co-clustering of MYC pathway with Chemokine, Cytokine, IL-6, JAK STAT 3 signaling, and IgA pathways (FIG. 10C), demonstrating their potential cross-talk in CRPC setting.

### Network Mining I: Identifying Differentially Altered Sub-Networks

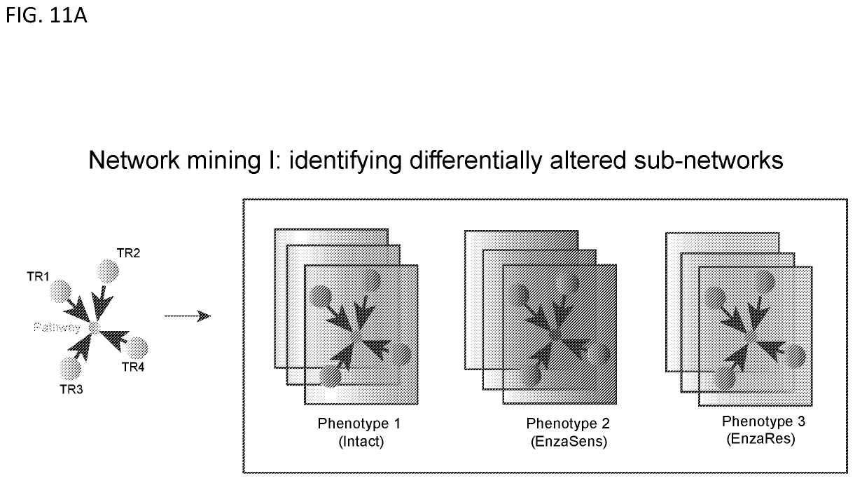

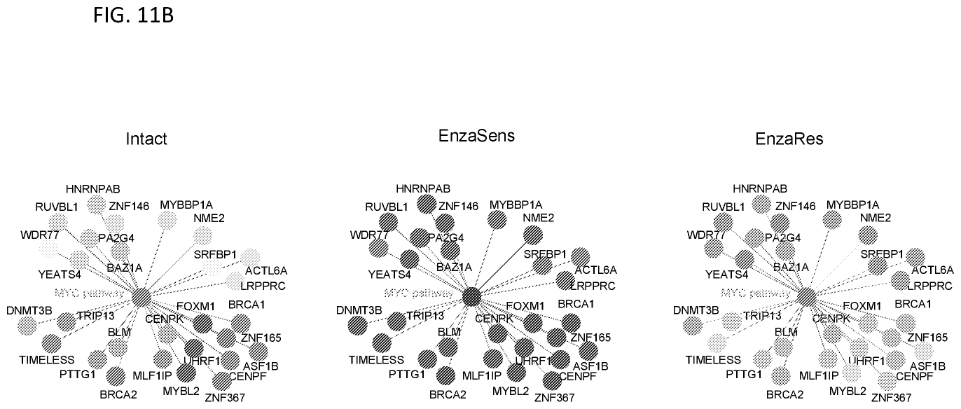

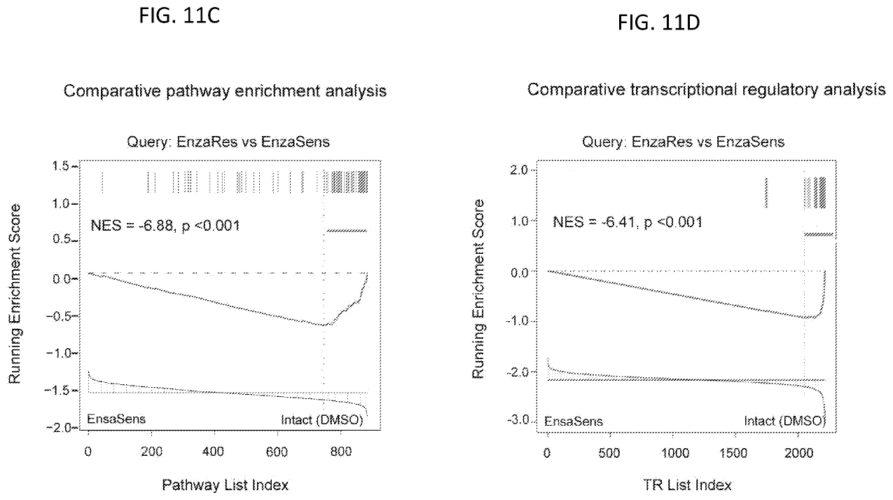

The next step was to utilize this network to identify TR programs upstream of MYC pathway that are involved in Enzalutamide response and resistance. To achieve this, we aimed to identify parts (sub-networks) of the mechanism-centric network that significantly change (alter) between phenotypes of interest, in our case—phenotypes that describe response to Enzalutamide (FIG. 11A). To accurately capture response and resistance to Enzalutamide in a controlled setting, we utilized gene expression profiles from Kregel et al. (Table 1, n=12), which is based on the LNCaP experimental system that has been widely used to study Enzalutamide-resistance previously. These profiles included (i) LNCaP parental intact samples subjected to DMSO (phenotype 1—Intact, FIG. 11A); (ii) LNCaP samples treated for 48 hours with Enzalutamide, where their survival and proliferation was sensitive to Enzalutamide (phenotype 2—EnzaSens, FIG. 11A; and (iii) LNCaP samples treated with Enzalutamide for 6 months, where their survival and proliferation did not depend on Enzalutamide (phenotype 3—EnzaRes, FIG. 11A).

We hypothesized that regulatory programs that are active in the intact state, then are repressed by Enzalutamide treatment (EnzaSens phenotype) and further re-activated as Enzalutamide resistance develops (EnzaRes phenotype) would be effective candidates to uncover mechanisms that govern Enzalutamide-resistance (FIG. 11A). Such network mining (using pairwise phenotype comparison, FIGS. 11C-11D) identified TR mechanisms (n=28) upstream of the MYC pathway, which constitutes of MYC-centric TR sub-network with a significant “active->repressed->reactivated” activity changes across the Enzalutamide-related phenotypes (FIG. 11B).

### Network Mining II: Prioritization of Upstream Regulatory Programs

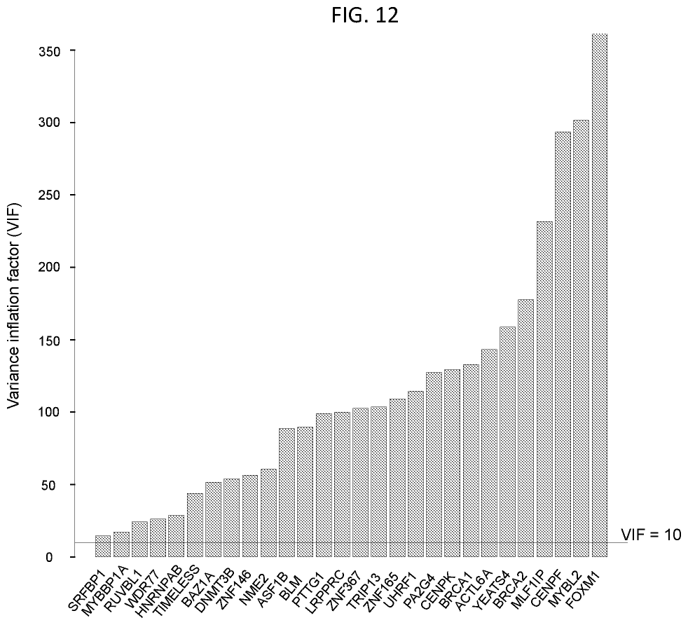

The next step was to prioritize the identified transcriptional regulatory programs upstream of a pathway of interest (e.g., MYC pathway) for experimental validation and potential salvage therapeutic targeting. We developed such a prioritization step to overcome several important drawbacks, commonly present in widely utilized statistical analyses. First of all, our method considers potential multi-collinearity among input variables (TRs), which is often naturally present in biological systems, yet can substantially obstruct statistical learning. In fact, variance inflation factor (VIF) analysis of the 28 TR programs identified upstream of MYC demonstrated significant multi-collinearity (all TRs had VIF>10, a multi-collinearity threshold suggested in Vatcheva et al. (Epidemiology (Sunnyvale) 6:227, 2016 and Kim (Korean J. Anesthesiol. 72:558-569, 2019)) (FIG. 12) and thus requires special methods to avoid information loss or model misinterpretation. Commonly utilized methods for handling multi-collinearity (e.g., regularization techniques), often keep one of the collinear variables in the model and eliminate others at random, thus limiting biological interpretability and translatability of the model. Our technique keeps all input variables intact, instead identifying their potential groups (based on their effect on the pathway of interest) and preventing information loss. Furthermore, our prioritization method not only test for direct regulatory relationships but also considers that a meaningful regulatory relationship can exist between entities that do not necessarily have direct (but rather indirect) interactions, which are widely present in biological systems, potentially including relationships between TRs and biological pathways.

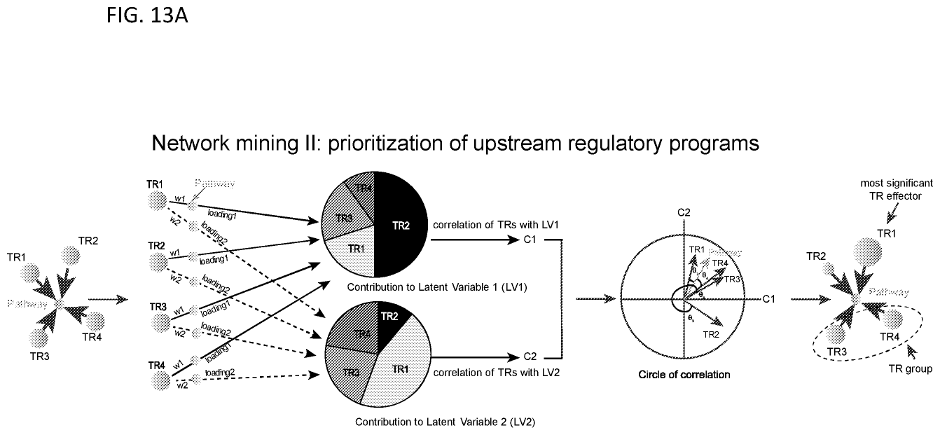

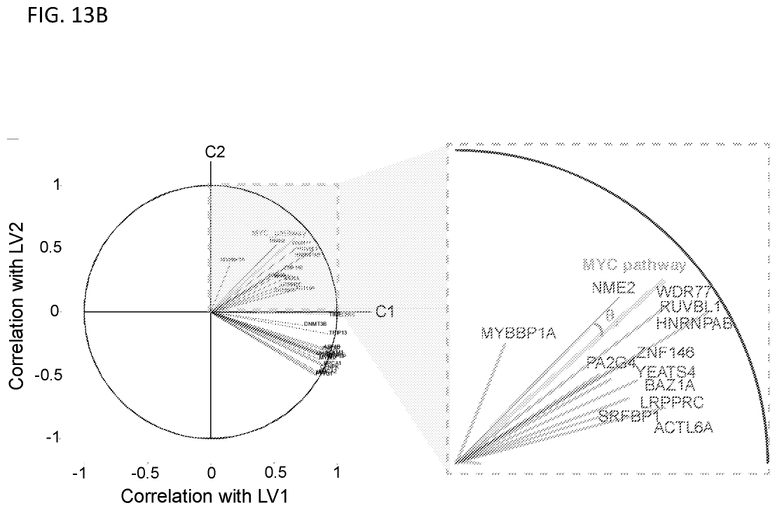

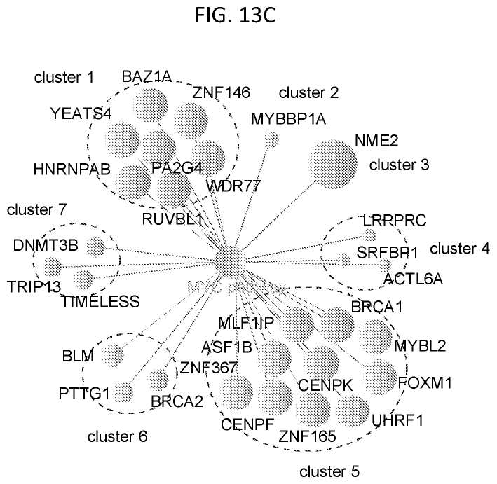

To overcome these limitations, we developed a prioritization step, inspired by the Partial Least Squares (PLS) approach, which has been mostly utilized in social sciences (sometimes referred to as a “supervised PCA”) but has not been used for network-based mining in oncology to date. Briefly, to prioritize the effect of the TR programs on the MYC pathway, our approach considers TR activity vectors as predictor (input) variables and utilizes MYC pathway activity vector as a response (output) variable (FIG. 13A, left). It then regresses TR activity vectors on the MYC pathway vector so that a linear combination of TRs defines a latent variable (FIG. 13A, left). This latent variable is then “subtracted” from the TR activity vectors, leaving the residuals to be utilized for defining the next latent variable. Identified latent variables do not express collinearity or multi-collinearity and are utilized as axes to build a “circle of correlation” (FIG. 13A, middle). Such a circle of correlation depicts the association of TR programs and the MYC pathway (defined as arrows on the circle of correlation, see Methods) to each latent variable. We then defined a method that utilized unsupervised hierarchical clustering on the degree of closeness (angle) between TR and pathway arrows so that TRs in high proximity to one another (thus having similar effects on latent variables) are grouped as they express simultaneous effect on the MYC pathway (FIG. 13A, right). Such TR groups/clusters (which also include groups with one TR) are then “prioritized” based on their effect on the MYC pathway activity (FIG. 13A, right) using effect scores, which are defined as a combination of (i) degree of closeness between a TR group/cluster and the MYC pathway on the circle of correlation (angle between their arrows), which reflects effect of each TR group activity changes on MYC pathway; (ii) association (Pearson correlation) between a TR group/cluster and each evaluated latent variable, which reflects contribution of each TR group to each latent variable; and (iii) edge weight between TR group/cluster and the MYC pathway from the TR-2-PATH mechanism-centric network reconstruction step, which reflects robustness of their regulatory relationship (FIG. 13B).

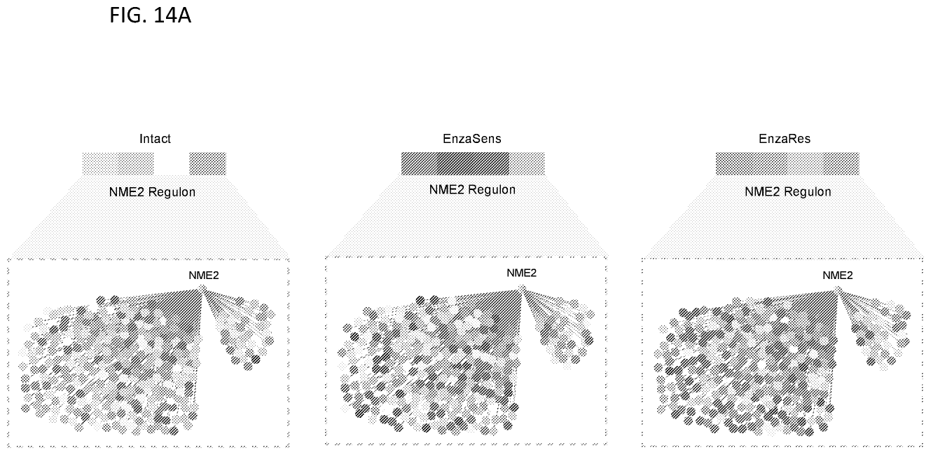

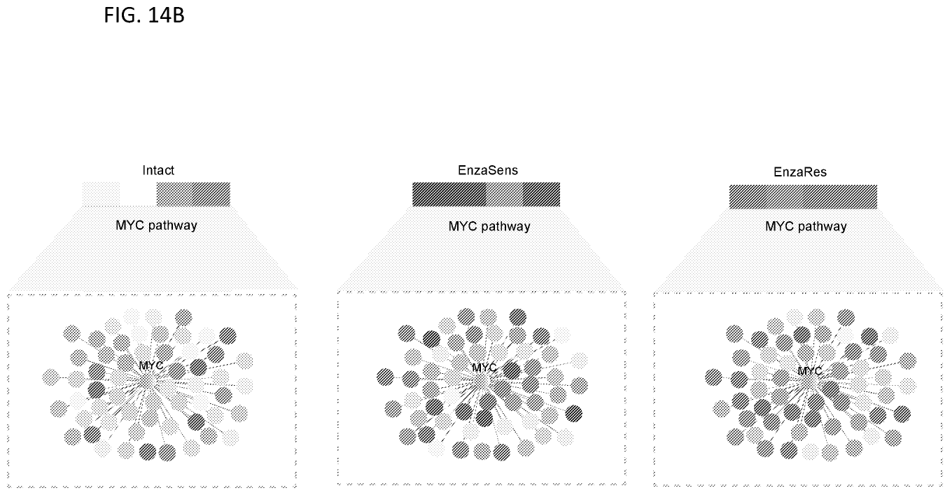

This approach identified 7 TR groups/clusters, based on their effect on the MYC pathway activity (two of the clusters had single TRs, FIG. 13C): group/cluster 1 (HNRNPAB, YEATS4, BAZ1A, ZNF146, WDR77, RUVBL1, and PA2G4), group/cluster 2 (MYBBP1A), group/cluster 3 (NME2), group/cluster 4 (ACTL6A, LRPPRC and SRFBP1), group/cluster 5 (FOXM1, MYBL2, BRCA1, MLF1IP, ASF1B, ZNF367, CENPF, ZNF165, CENPK, and UHRF1), group/cluster 6 (BRCA2, PTTG1, and BLM), and group/cluster 7 (TIMELESS, TRIP13, and DNMT3B) (FIG. 13C). Each group/cluster was then assigned an effect score, with group/cluster 3 (NME2) having the highest effect (score) on the MYC pathway (FIG. 13C, FIGS. 14A-14B, Table 2). While our analysis nominated NME2 transcriptional regulatory program to have the highest effect on MYC pathway in Enzalutamide-associated CRPC context, it has also been previously shown to bind to the MYC promoter region and upregulate MYC transcription, suggesting that further investigation of this relationship may uncover aspects of the transcriptional regulatory mechanisms governing the MYC pathway which could potentially provide an additional axis for therapeutic targeting for CRPC patients.

TABLE 2
Prioritization of transcriptional regulatory programs affecting MYC pathway

| clusters of collinear/ | correlation of | | correlation of | | | | | | |
|---|---|---|---|---|---|---|---|---|---|
| stand alone | transcriptional | | transcriptional | | angle | | | | |
| transcriptional | regulatory | | regulatory | | with | | | | |
| regulatory | programs | | programs | | respect | | | | |
| programs affecting | with latent | | with latent | | to MYC | | edge | | rank |
| MYC pathway | variable 1 | rank | variable 2 | rank | pathway | rank | weight | rank | product |
| Cluster 3 (NME2) | 0.52 | 6 | 0.53 | 1 | 4.18 | 1 | 100.00 | 1 | 1.57 |
| Cluster 5 | 0.89 | 1 | −0.35 | 6 | 19.76 | 4 | 100.00 | 1 | 2.21 |
| Cluster 1 | 0.63 | 4 | −0.10 | 5 | 10.86 | 2 | 97.14 | 2 | 2.99 |
| Cluster 7 | 0.87 | 2 | 0.49 | 7 | 34.83 | 7 | 100.00 | 1 | 3.15 |
| Cluster 6 | 0.84 | 3 | 0.18 | 4 | 11.02 | 3 | 96.67 | 3 | 3.22 |
| Cluster 2 (MYBBP1A) | 0.16 | 7 | 0.38 | 3 | 26.37 | 6 | 100.00 | 1 | 3.35 |
| Cluster 4 | 0.57 | 5 | 0.38 | 2 | 23.69 | 5 | 91.67 | 4 | 3.76 |

### Validation in Clinical Cohorts and Enzalutamide-Specificity

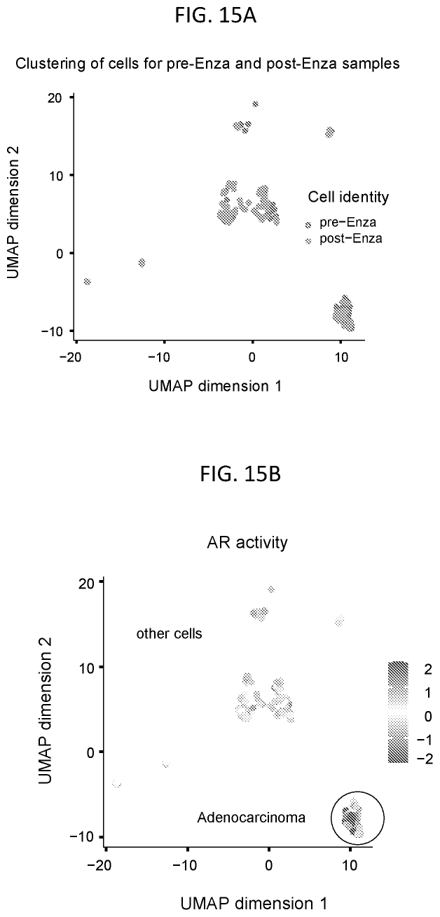

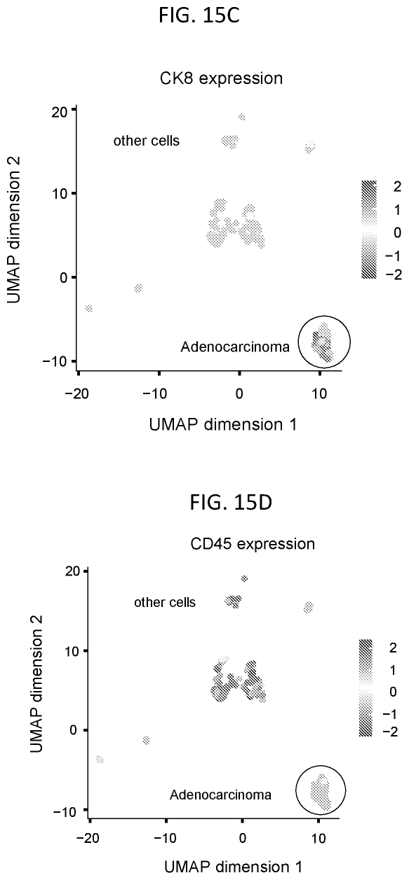

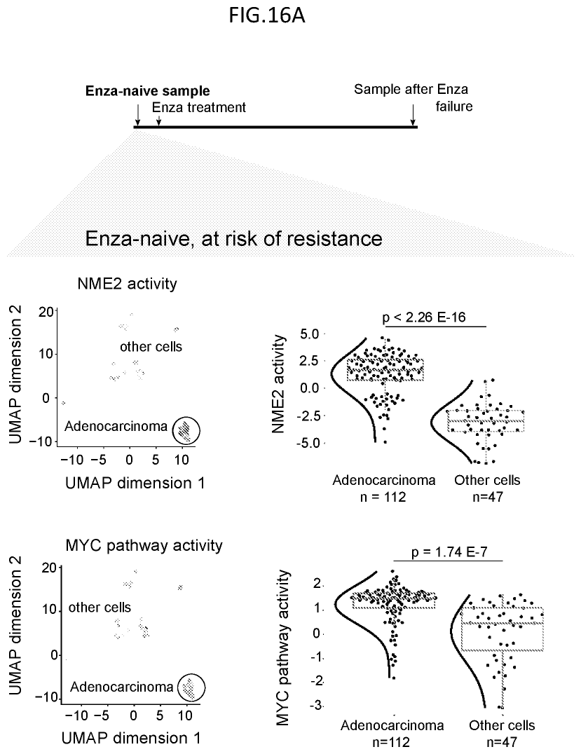

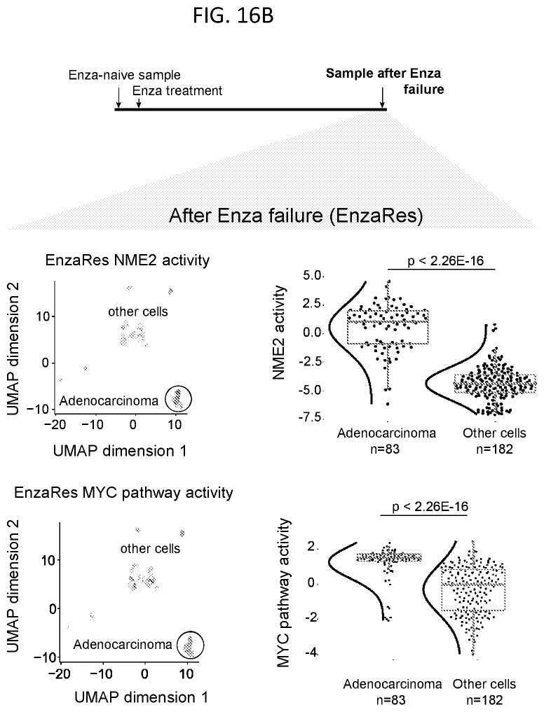

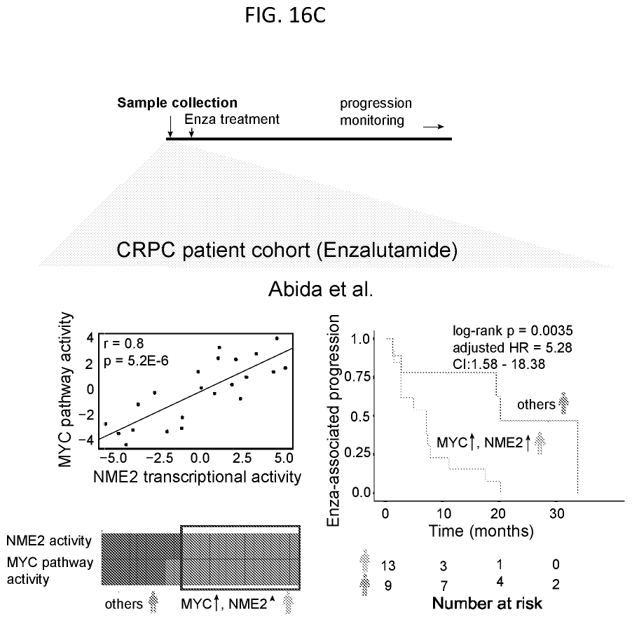

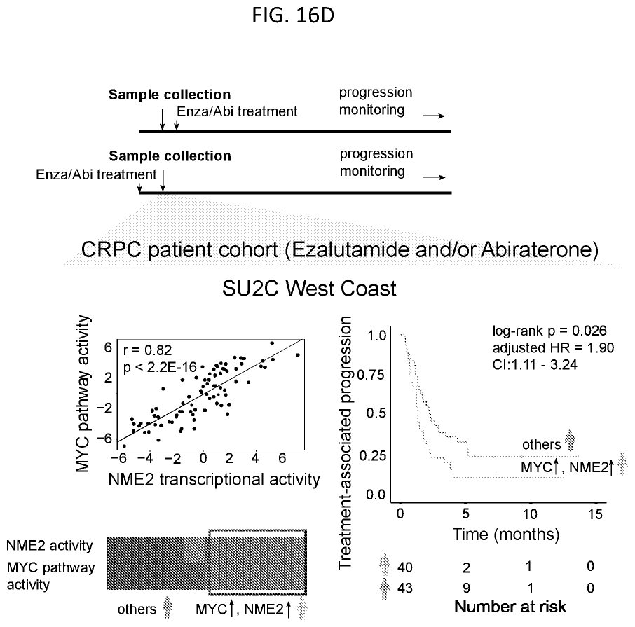

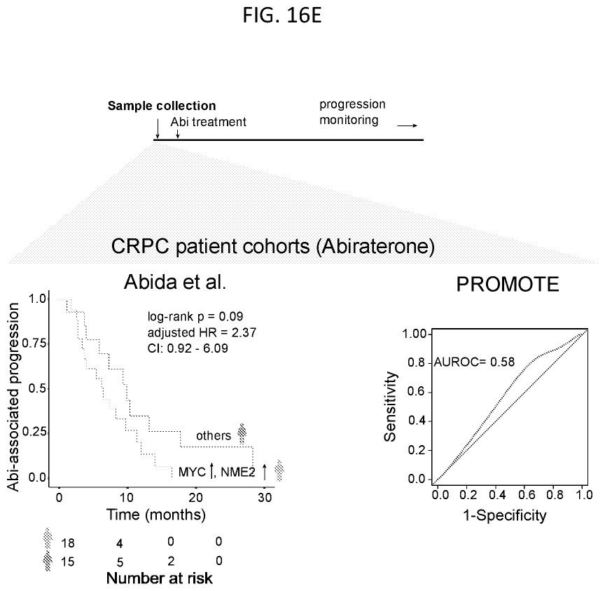

We next sought to confirm and evaluate if activation of NME2 TR program and MYC pathway are present in patients at risk of resistance to Enzalutamide and if they can be used as markers to risk-stratify patients prior to Enzalutamide administration. First, to evaluate if activity of the MYC pathway and NME2 TR program are high in treatment-naïve patients, who are at the risk of developing resistance to Enzalutamide, we have evaluated single-cell profiles from two sequential samples from a CRPC patient (01115655) that eventually failed Enzalutamide: neoadjuvant sample (prior to Enzalutamide treatment, FIG. 16A) and adjuvant sample (after developing resistance to Enzalutamide, EnzaRes, FIG. 16B) from He et al. (Table 1). After subjecting single-cell transcriptomic data to unsupervised uniform manifold approximation and projection (UMAP) clustering, to identify adenocarcinoma cells among the cell populations we assessed activity levels of AR, alongside expression of CK8 and CD45 (FIGS. 15A-15D). Following adenocarcinoma identification, we evaluated activity levels of the MYC pathway and NME2 TR program in both neoadjuvant and adjuvant samples. Our analysis indicated significantly higher levels of both NME2 TR activity and MYC pathway activity in adenocarcinoma cells, compared to other cells in Enzalutamide-naïve (neoadjuvant, FIG. 16A, one-tailed Welch t-test p-value<2.26E−16 for NME2 and p-value=1.74E−7 for MYC) and Enzalutamide-resistant, EnzaRes (adjuvant, FIG. 16B, one-tailed Welch t-test p-value<2.26E−16 for NME2 and p-value<2.26E−16 for MYC) samples, indicating that (i) both high-MYC pathway and high-NME2 TR activity levels were present prior to treatment in a patient who was at risk of developing subsequent resistance to Enzalutamide, nominating them as markers to identify patients at potential risk of Enzalutamide resistance; and (ii) both high-MYC pathway and high-NME2 TR activity levels were also observed after resistance to Enzalutamide developed (similar to our observation in LNCaP and C42B cell lines, FIGS. 9A-9B, FIGS. 14A-14B), cautiously nominating a MYC-centered salvage therapeutic line for patients that fail Enzalutamide.

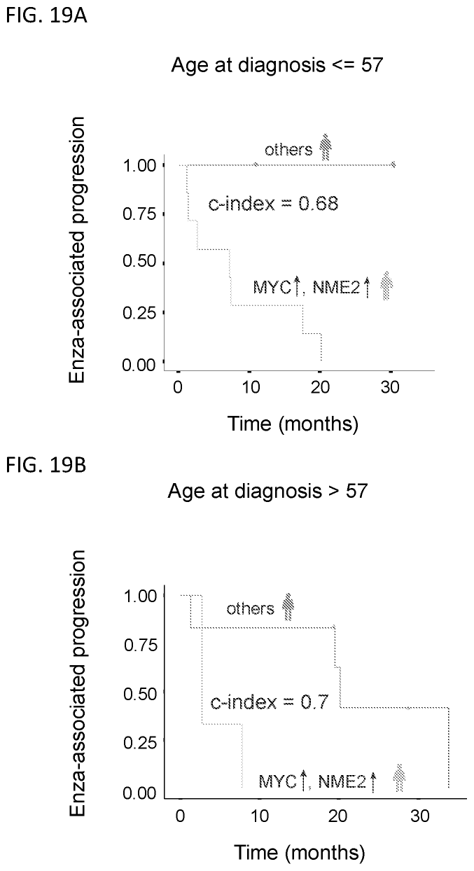

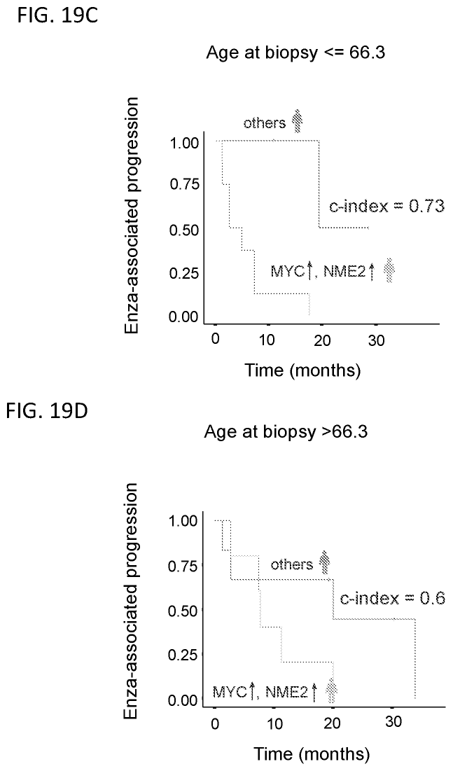

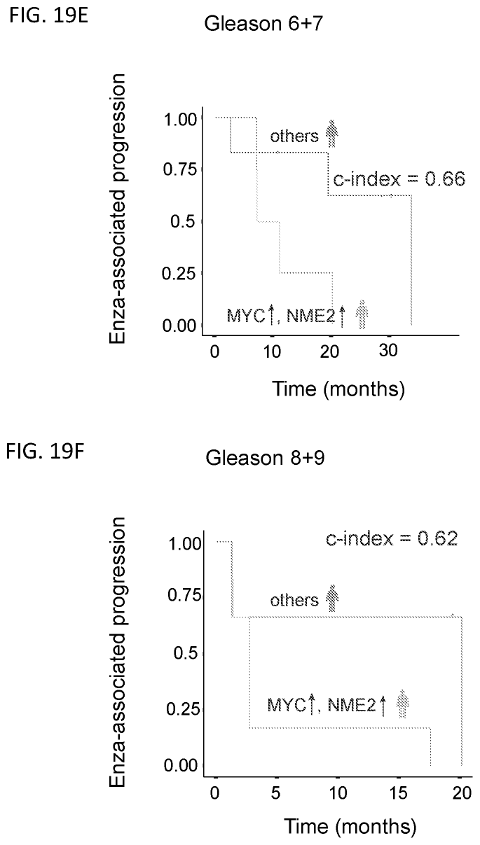

Given increased activity levels of both NME2 TR and MYC pathway in a single-cell sample from a treatment-naïve patient that later developed resistance to Enzalutamide, we sought to confirm their collective ability to predict CRPC patients at the treatment-naïve stage for risk of developing primary resistance to Enzalutamide using the Abida et al. cohort, which was also utilized in FIG. 9C (Table 1, n=22). As previously described, we used ARSI-naïve CRPC patients (that were later subjected to Enzalutamide and monitored for Enzalutamide-associated disease progression) to estimate NME2 TR and MYC pathway activity levels in each patient (see Methods). The NME2 TR and MYC pathway demonstrated striking concordance of their activity levels (FIG. 16C, left top, Pearson r=0.8, p-value=5.2E−6). We then subjected the activity levels of the NME2 TR and MYC pathway to unsupervised clustering (see Methods) that identified (i) patients with both high levels of NME2 transcriptional activity and MYC pathway activity (FIG. 16C, left bottom, n=13) and (ii) patients with at least one low/normal NME2 transcriptional activity and/or MYC pathway activity, categorized as “others” (FIG. 16C, left bottom, n=9). A comparison of these groups using Kaplan-Meier survival analysis and Cox proportional hazards model analysis, demonstrated a significant difference in their Enzalutamide-associated disease progression (FIG. 16C, right, log-rank p-value=0.0035, adjusted HR=5.28, CI=1.58-18.38, see Methods), suggesting that the high activity levels of NME2 TR and MYC pathway can be utilized to predict CRPC patients who are at a higher risk of developing resistance to Enzalutamide, prior to therapy administration. Next, to evaluate if our analysis is more applicable to specific patient sub-groups, we performed a stratified Kaplan-Meier survival analysis and separated patients by age at diagnosis (median age≤57 and >57), age at biopsy (median age≤66.3 and >66.3), and Gleason score (Gleason score 6+7 and 8+9) (FIGS. 19A-19F), and demonstrated that predictive ability of NME2 TR and MYC pathway was applicable in all stratified patient groups and was not significantly affected by these co-variates.

To extend our validation studies to an additional patient cohort, we utilized SU2C West Coast cohort (Table 1, n=83) which comprises of CRPC patients that were subjected to Enzalutamide and/or Abiraterone (complete separation of treatments was not possible according to correspondence with the dataset owners) either before or after biopsy (before or after sample collection) and were subsequently monitored for treatment-associated disease progression (which was defined in Quigley et al. and Aggrawal et al. as increase in PSA level (minimum 2 ng/mL) that has risen at least twice, in an interval of at least one week or soft tissue progression (nodal and visceral) based on RECIST v1.1). For each of these CRPC patients, we first estimated their NME2 TR and MYC pathway activity levels (see Methods) followed by evaluating association between NME2 TR and MYC pathway activity. Our analysis demonstrated striking concordance between NME2 TR and MYC pathway activity (FIG. 16D, left top, Pearson r=0.82, p-value<2.2E−16, see Methods). Next, we subjected the activity levels of the NME2 TR and MYC pathway to unsupervised clustering that identified (i) patients with both high NME2 TR and MYC pathway activity levels (FIG. 16D, left bottom, n=40) and (ii) patients with at least one low/normal NME2 TR and/or MYC activity levels, categorized as “others” (FIG. 16D, left bottom, n=43). A comparison of these groups using Kaplan-Meier survival analysis and Cox proportional hazards model analysis demonstrated a significant difference in treatment-associated disease progression (FIG. 16D, right, log-rank p-value=0.026, adjusted HR=1.90, CI=1.11-3.24, see Methods), which supports our previous observations.

To further investigate and confirm that the activation of NME2 TR and MYC pathway could specifically predict Enzalutamide-failure (and not, for example, Abiraterone-failure), we analyzed RNA-seq profiles from two Abiraterone-specific cohorts: (i) ARSI-naïve CRPC patients from Abida et at. that were subjected to Abiraterone after sample collection and monitored for Abiraterone-associated disease progression, as in FIG. 9D (Table 1, n=33) and (ii) PROMOTE cohort, which is comprised of ARSI-naïve CRPC patients, subjected to Abiraterone for 12 weeks after sample collection and then assessed for disease progression (binary outcomes, where disease progression was defined based on the combined score that included serum PSA level, bone and CT imaging and symptom assessments at week 12, Table 1, n=77). In both cohorts, we estimated the NME2 TR and MYC pathway activity in each sample and subjected them to similar analyses as above. Kaplan-Meier survival analysis' and Cox proportional hazards model analysis on the Abida et al. cohort demonstrated no significant difference in Abiraterone-associated disease progression between the two identified patient groups (FIG. 16E left, log-rank p-value=0.09, adjusted HR=2.37, CI=0.92-6.09). ROC analysis in the PROMOTE cohort demonstrated that activation of NME2 and MYC did not classify patients based on their binary response to Abiraterone (FIG. 16E, right, AUROC=0.58, where AUROC=0.5 indicates a random classifier), demonstrating that partnership between NME2 TR and MYC pathway is specifically indicative of the risk of developing resistance to Enzalutamide and not Abiraterone.

### Comparison to Common Markers of PCa Aggressiveness and Treatment Response

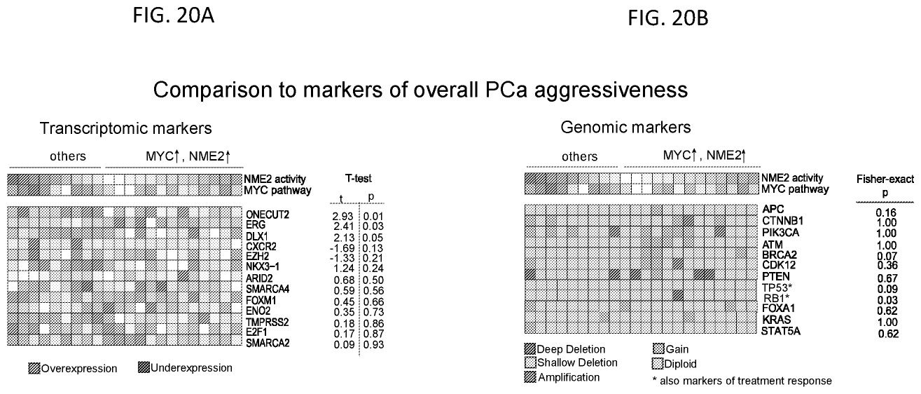

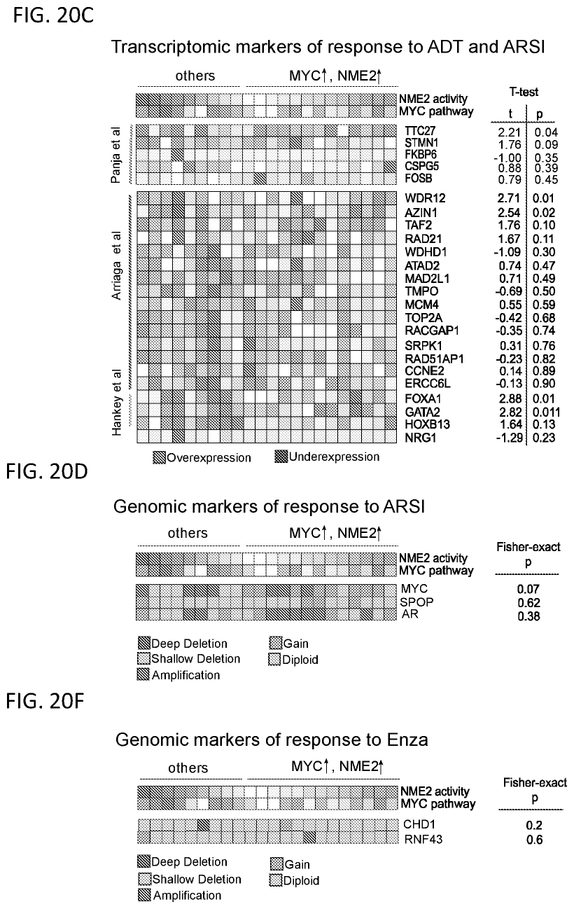

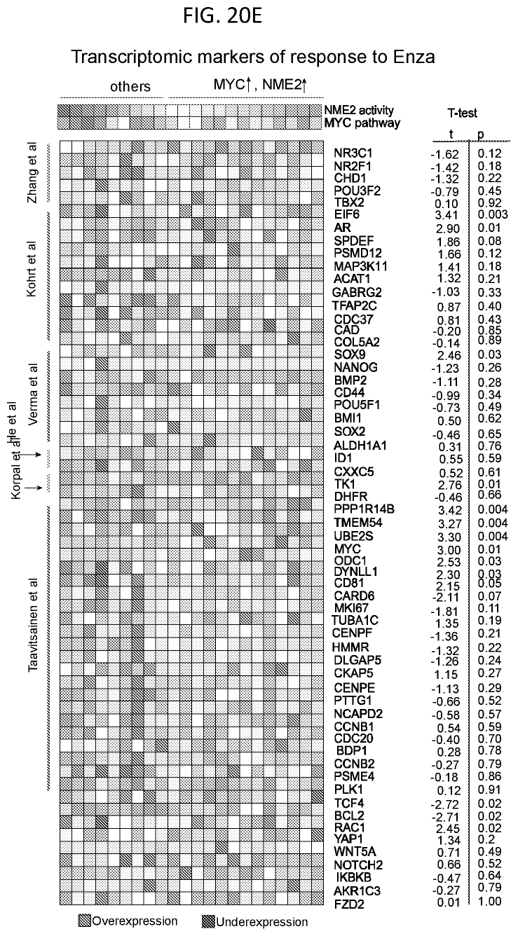

We next compared the ability of NME2 TR and MYC pathway to predict Enzalutamide resistance in Abida et al. cohort, with the predictive ability of known markers of prostate cancer (i) aggressiveness, (ii) response to first-generation ADT and ARSIs (not specific to any particular drug), and (iii) Enzalutamide-specific response (FIGS. 20A-20F). Transcriptomic and genomic markers were considered separately. Comparison to transcriptomic markers of PCa aggressiveness (FIG. 20A), demonstrated that three markers (ONECUT2, ERG, and DLX1) showed significant differential expression in the high-MYC and high-NME2 group, compared to the rest of the patients, “others” (two-tailed Welch t-test p-value<0.05, Table 3). Interestingly, all three have a direct relationship to MYC: ONECUT2 is a known transcriptional target of MYC, ERG fusion (which eventually leads to overexpression of ERG) was shown to be correlated to MYC expression, and DLX1 is a known transcriptional target of ERG (FIG. 20A). To assess an independent association of the transcriptomic markers to Enzalutamide resistance (independent of MYC and NME2), we have performed a univariable Cox proportional hazards model analysis, using Enzalutamide-associated disease progression as the end-point in Abida et al. cohort, which showed no significant association of these markers to Enzalutamide resistance (Table 3). From genomic markers of PCa aggressiveness (FIG. 20B, where TP53 and RB1 have also been shown to be markers of response to ARSIs), RB1 with shallow and deep deletion was significantly enriched in patients with high-NME2 and high-MYC pathway activity (Fisher's exact test p-value=0.03) (FIG. 20B, Table 4). Interestingly, RB1 loss has been shown to correlate with MYC expression in small cell lung carcinoma, indicating potential cross-talk with the MYC pathway. Independent Cox proportional hazards model analysis identified a shallow and deep deletion of KRAS to be significantly associated with response to Enzalutamide (Wald p-value=0.01, Table 4), which has also been previously shown to be associated with MYC.

Comparison to transcriptomic markers of first-generation ADT and ARSIs (taken from Panja et al. (EBioMedicine 31:110-121, 2018), Arriaga et al. (Nature Cancer 1:1082-1096, 2020), Hankey et al. (Cancer Research 80:2427-2436, 2020), and Zhang et al. (Cancer Cell 38:279-296, 2020)), demonstrated that five markers (TTC27, WDR12, AZIN1, FOXA1, and GATA2) were significantly differentially expressed in the high-MYC and high-NME2 patients (two-tailed Welch t-test p-value<0.05, Table 5, FIG. 20C). Interestingly, WDR12 and AZIN1 are members of the MYC pathway and TTC27, FOXA1, and GATA2 are MYC transcriptional targets. Furthermore, Cox proportional hazards model analysis³⁷ indicated that five of the transcriptomic markers (STMN1, WDR12, AZIN1, MAD2L1, and MCM4) had a significant association with response to Enzalutamide (Wald p-value<0.05, Table 5), yet many of them were borderline significant and did not outperform MYC and NME2 (Table 5). Additionally, genomic markers of first-generation ADT and ARSIs described in Arriaga et al. and Abida et al., had no significant enrichment in the high-MYC and high-NME2 group (FIG. 20D, Table 6) or independent response to Enzalutamide.

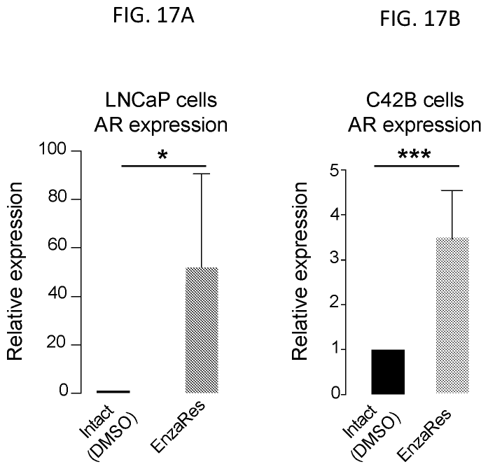

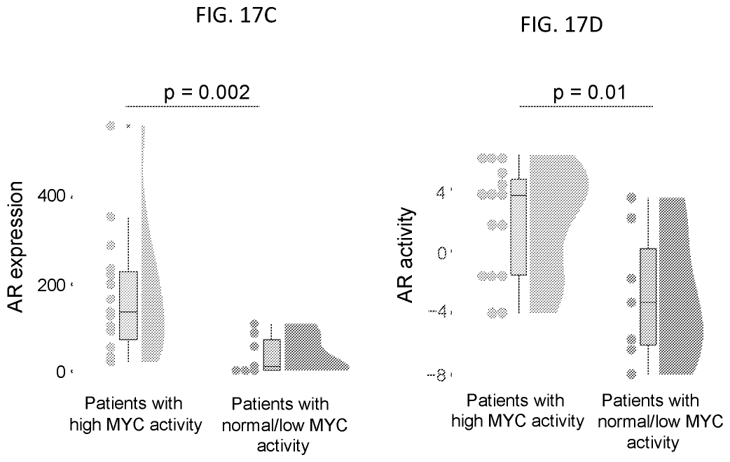

Comparison to transcriptomic markers of Enzalutamide-specific response (described by Zhang et al (Cancer Cell 38:584-598, 2020), Kohrt et al. (Mol. Cancer Ther. 20:398-409, 2021), Verma et al. (Int. J. Mol. Sci. 21, doi:10.3390/ijms21249568, 2020), He et al. (Nucleic Acids Res. 46:1895-1911, 2018), Korpal et al. (Cancer Discov. 3:1030-1043, 2013), Taavitsainen et al. (Nature Commun. 12:5307, 2021), in addition to others) demonstrated that a group of 14 markers (EIF6, AR, SOX9, TK1, PPP1R14B, TMEM54, UBE2S, MYC, ODC1, DYNLL1, CD81, BCL2, TCF4 and RAC1) had significant differential expression in the high-MYC and high-NME2 group (two-tailed Welch t-test p-value<0.05, Table 7, FIG. 20E). Interestingly, 12 of these (EIF6, SOX9, TK1, PPP1R14B, TMEM54, UBE2S, MYC, ODC1, DYNLL1, CD81, BCL2, and TCF4) were transcriptional MYC targets, determined from ChEA transcription factor targets dataset. Another member of this group, AR, as we have shown previously (FIG. 17C), was also differentially expressed between the groups. Finally, RAC1 (FIG. 20E, Table 7) is a member of the RAS pathway which has been shown to be associated with MYC pathway in CRPC samples. Cox proportional hazards model analysis³⁷ demonstrated that 10 of the transcriptomic markers (EIF6, ACAT1, TK1, PPP1R14B, TMEM54, UBE2S, DYNLL1, TUBA1C, RAC1, and WNT5A) had significant association with response to Enzalutamide (Wald p-value<0.05, Table 7), yet many of them were border-line significant and none of them outperformed NME2 and MYC (Table 7). None of the genomic markers of Enzalutamide-specific response (described by Zhang et al. and Guan et al. (Clin Cancer Res 26:3616-4624, 2020)) were significantly enriched in the high-MYC and high-NME2 group (FIG. 20F, Table 8) or independently associated with response to Enzalutamide. Taken together, these findings indicate that the majority of the markers of PCa aggressiveness and therapeutic response that are enriched in the high-MYC and high-NME2 group are associated with MYC-related mechanisms and none of them outperform the ability of MYC and NME2 to predict risk of Enzalutamide resistance.

TABLE 3
Comparison to known markers of overall prostate cancer aggressiveness

| | | | Comparison of | | | | | |
|---|---|---|---|---|---|---|---|---|
| | | | known markers | | Association of known markers to | | | |
| | Progression | | between groups | | Enzalutamide associated progression | | | |
| Human | related | Publication | T-test | T-test | Hazard | | | |
| entrez | markers | PubMed Id | tvalue | p | ratio | lower_95 | upper_95 | Cox p |
| 9480 | ONECUT2 | PMID: 30478421 | 2.93 | 0.01* | 1.15 | 0.72 | 1.83 | 0.57 |
| 2078 | ERG | PMID: 16254181, | 2.41 | 0.03* | 1.00 | 0.99 | 1.02 | 0.78 |
| | | PMID: 19584279 | | | | | | |
| 1745 | DLX1 | PMID: 34493733 | 2.13 | 0.05* | 1.00 | 0.96 | 1.04 | 0.98 |
| 3579 | CXCR2 | PMID: 31801883 | −1.69 | 0.13 | 0.98 | 0.95 | 1.02 | 0.38 |
| 2146 | EZH2 | PMID: 34489575 | −1.33 | 0.21 | 1.01 | 0.95 | 1.08 | 0.78 |
| 4824 | NKX3-1 | PMID: 11085535 | 1.24 | 0.24 | 1.00 | 1.00 | 1.00 | 0.48 |
| 196528 | ARID2 | PMID: 34163028 | 0.68 | 0.50 | 0.96 | 0.86 | 1.09 | 0.55 |
| 6597 | SMARCA4 | PMID: 33144576 | 0.59 | 0.56 | 0.97 | 0.93 | 1.02 | 0.22 |
| 2305 | FOXM1 | PMID: 30719174 | 0.45 | 0.66 | 1.06 | 0.99 | 1.13 | 0.07 |
| 2026 | ENO2 | PMID: 31547070 | 0.35 | 0.73 | 1.03 | 0.97 | 1.09 | 0.33 |
| 7113 | TMPRSS2 | PMID: 19584279 | 0.18 | 0.86 | 1.00 | 1.00 | 1.00 | 0.77 |
| 1869 | E2F1 | PMID: 31802906 | 0.17 | 0.87 | 1.08 | 0.99 | 1.18 | 0.10 |
| 6595 | SMARCA2 | PMID: 33144576 | 0.09 | 0.93 | 0.95 | 0.87 | 1.03 | 0.23 |

*significant p-value <0.05

TABLE 4
Comparison to known genomic markers of overall prostate cancer aggressiveness

| | Progression | | Deletion (both Shallow and Deep) | | | | | Gain and Amplification | |
|---|---|---|---|---|---|---|---|---|---|
| Human | related | Publication | Fisher | Hazard | lower_ | upper_ | Cox | Fisher | Hazard |
| entrez | markers | PubMed Id | exact p | ratio | 95 | 95 | p | exact p | ratio |
| 324 | APC | PMID: 26000489, | 0.33 | 1.24 | 0.43 | 3.57 | 0.68 | 1 | 0.64 |
| 1499 | CTNNB1 | PMID: 29610475, | 1 | NA | NA | NA | NA | 1 | 0.76 |
| 5290 | PIK3CA | PMID: 30928160 | 1 | NA | NA | NA | NA | 1 | 1.42 |
| 472 | ATM | | 0.15 | 1.3 | 0.29 | 5.81 | 0.72 | 0.11 | 1.94 |
| 675 | BRCA2 | | 0.36 | 1.09 | 0.35 | 3.38 | 0.87 | 0.24 | 1.46 |
| 51755 | CDK12 | | 1 | 9.7E+09 | 0 | Inf | 0.99 | 0.62 | 0.71 |
| 5728 | PTEN | PMID: 26299074 | 1 | 1.33 | 0.51 | 3.49 | 0.55 | 1 | 0.64 |
| 7157 | TP53 | | 0.09 | 2.1 | 0.79 | 5.57 | 0.13 | 1 | NA |
| 5925 | RB1 | | 0.03* | 1.89 | 0.69 | 5.18 | 0.21 | 1 | NA |
| 3169 | FOXA1 | | 1 | 0.64 | 0.08 | 4.94 | 0.67 | 1 | 0.72 |
| 3845 | KRAS | | 0.62 | 5.48 | 1.5 | 19.93 | 0.01* | 1 | 2.48 |
| 6776 | STAT5A | PMID: 31292140 | 0.24 | 3.34 | 0.85 | 0.001 | 0.08 | 0.41 | 1.20E−08 |
| | | Gain and Amplification | | | Diploid | | | | |
| | Human | lower_ | upper_ | Cox | Fisher | Hazard | lower_ | upper_ | Cox |
| | entrez | 95 | 95 | p | exact p | ratio | 95 | 95 | p |
| | 324 | 0.08 | 4.94 | 0.67 | 0.16 | 0.94 | 0.34 | 2.55 | 0.9 |
| | 1499 | 0.21 | 2.66 | 0.66 | 1 | 1.31 | 0.37 | 4.61 | 0.66 |
| | 5290 | 0.54 | 3.78 | 0.47 | 1 | 0.69 | 0.26 | 1.85 | 0.47 |
| | 472 | 0.62 | 5.99 | 0.24 | 1 | 0.54 | 0.19 | 1.48 | 0.23 |
| | 675 | 0.41 | 5.12 | 0.55 | 0.07 | 0.75 | 0.28 | 2 | 0.57 |
| | 51755 | 0.2 | 2.51 | 0.59 | 0.36 | 0.98 | 0.31 | 3.06 | 0.97 |
| | 5728 | 0.08 | 4.94 | 0.67 | 0.67 | 0.83 | 0.31 | 2.21 | 0.72 |
| | 7157 | NA | NA | NA | 0.09 | 0.47 | 0.17 | 1.25 | 0.13 |
| | 5925 | NA | NA | NA | 0.03* | 0.52 | 0.19 | 1.44 | 0.21 |
| | 3169 | 0.16 | 3.19 | 0.67 | 0.62 | 1.5 | 0.42 | 5.29 | 0.52 |
| | 3845 | 0.52 | 11.86 | 0.25 | 1 | 0.16 | 0.04 | 0.59 | 0.005* |
| | 6776 | 0 | Inf | 0.99 | 0.62 | 0.9 | 0.25 | 3.16 | 0.87 |

| | | | Comparison of | | | | | |
|---|---|---|---|---|---|---|---|---|
| | Second | | known markers | | Association of known markers to | | | |
| | generation | | between groups | | Enzalutamide associated progression | | | |
| Human | related | Publication | T-test | T-test | Hazard | | | |
| entrez | markers | PubMed Id | tvalue | p | ratio | lower_95 | upper_95 | Cox p |
| 55622 | TTC27 | PMID: 29685789 | 2.21 | 0.04* | 1.14 | 0.98 | 1.34 | 0.09 |
| 3925 | STMN1 | | 1.76 | 0.09 | 1.02 | 1.01 | 1.03 | 0.002* |
| 8468 | FKBP6 | | −1.00 | 0.35 | 0.00 | 0.00 | 296.57 | 0.29 |
| 10675 | CSPG5 | | 0.88 | 0.39 | 1.11 | 0.95 | 1.29 | 0.20 |
| 2354 | FOSB | | 0.79 | 0.45 | 1.00 | 0.96 | 1.05 | 1.00 |
| 55759 | WDR12 | PMID: 34085047 | 2.71 | 0.01* | 1.27 | 1.06 | 1.52 | 0.01* |
| 51582 | AZIN1 | | 2.54 | 0.02* | 1.02 | 1.00 | 1.03 | 0.03* |
| 6873 | TAF2 | | 1.76 | 0.10 | 1.02 | 0.95 | 1.10 | 0.60 |
| 5885 | RAD21 | | 1.67 | 0.11 | 1.01 | 1.00 | 1.02 | 0.12 |
| 11169 | WDHD1 | | −1.09 | 0.30 | 1.05 | 0.88 | 1.26 | 0.58 |
| 29028 | ATAD2 | | 0.74 | 0.47 | 1.05 | 1.00 | 1.10 | 0.06 |
| 4085 | MAD2L1 | | 0.71 | 0.49 | 1.13 | 1.04 | 1.24 | 0.01* |
| 7112 | TMPO | | −0.69 | 0.50 | 1.01 | 0.98 | 1.05 | 0.48 |
| 4173 | MCM4 | | 0.55 | 0.59 | 1.03 | 1.00 | 1.05 | 0.03* |
| 7153 | TOP2A | | −0.42 | 0.68 | 1.01 | 0.99 | 1.02 | 0.32 |
| 29127 | RACGAP1 | | −0.35 | 0.74 | 1.03 | 0.99 | 1.07 | 0.15 |
| 6732 | SRPK1 | | 0.31 | 0.76 | 1.01 | 0.98 | 1.04 | 0.46 |
| 10635 | RAD51AP1 | | −0.23 | 0.82 | 1.07 | 0.98 | 1.16 | 0.11 |
| 9134 | CCNE2 | | 0.14 | 0.89 | 1.07 | 0.93 | 1.22 | 0.34 |
| 54821 | ERCC6L | | −0.13 | 0.90 | 1.32 | 0.87 | 2.01 | 0.19 |
| 3169 | FOXA1 | PMID: 32094298 | 2.88 | 0.01* | 1.00 | 1.00 | 1.01 | 0.22 |
| 2624 | GATA2 | | 2.82 | 0.011* | 1.01 | 0.99 | 1.02 | 0.42 |
| 10481 | HOXB13 | | 1.64 | 0.13 | 1.00 | 1.00 | 1.01 | 0.23 |
| 3084 | NRG1 | PMID: 32679108 | −1.29 | 0.23 | 0.48 | 0.08 | 3.03 | 0.43 |

*significant p-value <0.05

TABLE 6
Comparison to known genomic markers related with response to ARSI

| | Progression | | Deletion (both Shallow and Deep) | | | | | Gain and Amplification | |
|---|---|---|---|---|---|---|---|---|---|
| Human | related | Publication | Fisher | Hazard | lower_ | upper_ | Cox | Fisher | Hazard |
| entrez | markers | PubMed Id | exact p | ratio | 95 | 95 | p | exact p | ratio |
| 4609 | MYC | PMID: 34085047 | 1 | NA | NA | NA | NA | 0.07 | 1.79 |
| 8405 | SPOP | | 1 | NA | NA | NA | NA | 0.62 | 1.04 |
| 367 | AR | PMID: 31061129 | 1 | NA | NA | NA | NA | 0.38 | 1.45 |
| | | Gain and Amplification | | | Diploid | | | | |
| | Human | lower_ | upper_ | Cox | Fisher | Hazard | lower_ | upper_ | Cox |
| | entrez | 95 | 95 | p | exact p | ratio | 95 | 95 | p |
| | 4609 | 0.58 | 5.52 | 0.3 | 0.07 | 0.55 | 0.18 | 1.7 | 0.3 |
| | 8405 | 0.28 | 3.56 | 0.98 | 0.62 | 0.98 | 0.28 | 3.46 | 0.98 |
| | 367 | 0.55 | 4.05 | 0.42 | 0.38 | 0.66 | 0.24 | 1.81 | 0.42 |

TABLE 7
Comparison to known markers related with response to Enzalutamide

| | | | Comparison of | | | | | |
|---|---|---|---|---|---|---|---|---|
| | | | known markers | | Association of known markers to | | | |
| | Enzalutamide | | between groups | | Enzalutamide associated progression | | | |
| Human | related | Publication | T-test | T-test | Hazard | | | |
| entrez | markers | PubMed Id | tvalue | p | ratio | lower_95 | upper_95 | Cox p |
| 2908 | NR3C1 | PMID: 32220301 | −1.62 | 0.12 | 1.01 | 0.93 | 1.10 | 0.77 |
| 7025 | NR2F1 | | −1.42 | 0.18 | 0.82 | 0.67 | 1.01 | 0.06 |
| 1105 | CHD1 | | −1.32 | 0.22 | 0.94 | 0.80 | 1.11 | 0.47 |
| 5454 | POU3F2 | | −0.79 | 0.45 | 0.01 | 0.00 | 131.03 | 0.31 |
| 6909 | TBX2 | | 0.1 | 0.92 | 0.99 | 0.90 | 1.10 | 0.92 |
| 3692 | EIF6 | PMID: 33298586 | 3.41 | 0.003* | 1.03 | 1.01 | 1.05 | 0.003* |
| 367 | AR | | 2.9 | 0.01* | 1.00 | 1.00 | 1.01 | 0.05 |
| 25803 | SPDEF | | 1.86 | 0.08 | 1.00 | 1.00 | 1.00 | 0.98 |
| 5718 | PSMD12 | | 1.66 | 0.12 | 1.02 | 1.00 | 1.05 | 0.11 |
| 4296 | MAP3K11 | | 1.41 | 0.18 | 1.03 | 0.97 | 1.08 | 0.34 |
| 38 | ACAT1 | | 1.32 | 0.21 | 1.03 | 1.01 | 1.04 | 0.01* |
| 2566 | GABRG2 | | −1.03 | 0.33 | 0.00 | 0.00 | 249624.90 | 0.41 |
| 7022 | TFAP2C | | 0.87 | 0.4 | 0.98 | 0.90 | 1.07 | 0.63 |
| 11140 | CDC37 | | 0.81 | 0.43 | 1.00 | 0.99 | 1.01 | 0.84 |
| 790 | CAD | | −0.2 | 0.85 | 0.97 | 0.83 | 1.13 | 0.66 |
| 1290 | COL5A2 | | −0.14 | 0.89 | 1.00 | 0.97 | 1.03 | 0.85 |
| 6662 | SOX9 | PMID: 33339129 | 2.46 | 0.03* | 1.00 | 0.98 | 1.02 | 0.99 |
| 79923 | NANOG | | −1.23 | 0.26 | 0.00 | 0.00 | 163.01 | 0.15 |
| 650 | BMP2 | | −1.11 | 0.28 | 0.89 | 0.59 | 1.34 | 0.59 |
| 960 | CD44 | | −0.99 | 0.34 | 0.99 | 0.96 | 1.01 | 0.27 |
| 5460 | POU5F1 | | −0.73 | 0.49 | 0.90 | 0.68 | 1.19 | 0.47 |
| 648 | BMI1 | | 0.5 | 0.62 | 1.01 | 0.98 | 1.05 | 0.46 |
| 6657 | SOX2 | | −0.46 | 0.65 | 0.86 | 0.64 | 1.14 | 0.29 |
| 216 | ALDH1A1 | | 0.31 | 0.76 | 1.00 | 1.00 | 1.00 | 0.92 |
| 3397 | ID1 | PMID: 33750801 | 0.55 | 0.59 | 1.00 | 0.97 | 1.02 | 0.91 |
| 51523 | CXXC5 | | 0.52 | 0.61 | 1.02 | 0.96 | 1.08 | 0.51 |
| 7083 | TK1 | PMID: 23842682 | 2.76 | 0.01* | 1.03 | 1.01 | 1.05 | 0.01* |
| 1719 | DHFR | | −0.46 | 0.66 | 1.00 | 0.97 | 1.04 | 0.81 |
| 26472 | PPP1R14B | PMID: 34489465 | 3.42 | 0.004* | 1.01 | 1.00 | 1.03 | 0.02* |
| 113452 | TMEM54 | | 3.27 | 0.004* | 1.02 | 1.01 | 1.04 | 0.01* |
| 27338 | UBE2S | | 3.3 | 0.004* | 1.06 | 1.02 | 1.10 | 0.0031* |
| 4609 | MYC | | 3 | 0.01* | 1.01 | 1.00 | 1.02 | 0.16 |
| 4953 | ODC1 | | 2.53 | 0.03* | 1.00 | 1.00 | 1.00 | 0.28 |
| 8655 | DYNLL1 | | 2.3 | 0.03* | 1.03 | 1.02 | 1.05 | 0.0002* |
| 975 | CD81 | | 2.15 | 0.05* | 1.01 | 1.00 | 1.01 | 0.11 |
| 84674 | CARD6 | | −2.11 | 0.07 | 0.91 | 0.74 | 1.12 | 0.39 |
| 4288 | MKI67 | | −1.81 | 0.11 | 0.99 | 0.95 | 1.03 | 0.71 |
| 84790 | TUBA1C | | 1.35 | 0.19 | 1.01 | 1.00 | 1.03 | 0.04* |
| 1063 | CENPF | | −1.36 | 0.21 | 1.00 | 0.95 | 1.06 | 0.97 |
| 3161 | HMMR | | −1.32 | 0.22 | 1.02 | 0.97 | 1.07 | 0.47 |
| 9787 | DLGAP5 | | −1.26 | 0.24 | 1.00 | 0.97 | 1.03 | 0.92 |
| 9793 | CKAP5 | | 1.15 | 0.27 | 1.04 | 0.99 | 1.08 | 0.13 |
| 1062 | CENPE | | −1.13 | 0.29 | 1.01 | 0.91 | 1.12 | 0.79 |
| 9232 | PTTG1 | | −0.66 | 0.52 | 1.01 | 1.00 | 1.02 | 0.14 |
| 9918 | NCAPD2 | | −0.58 | 0.57 | 1.00 | 0.97 | 1.04 | 0.89 |
| 891 | CCNB1 | | 0.54 | 0.59 | 1.03 | 1.00 | 1.05 | 0.05 |
| 991 | CDC20 | | −0.4 | 0.7 | 1.01 | 0.99 | 1.04 | 0.31 |
| 55814 | BDP1 | | 0.28 | 0.78 | 1.00 | 0.90 | 1.11 | 0.99 |
| 9133 | CCNB2 | | −0.27 | 0.79 | 1.02 | 0.99 | 1.06 | 0.24 |
| 23198 | PSME4 | | −0.18 | 0.86 | 0.98 | 0.91 | 1.06 | 0.65 |
| 5347 | PLK1 | | 0.12 | 0.91 | 1.04 | 0.99 | 1.10 | 0.09 |
| 6925 | TCF4 | PMID: 31536510 | −2.72 | 0.02* | 0.89 | 0.70 | 1.14 | 0.37 |
| 596 | BCL2 | PMID: 31228231, | −2.71 | 0.02* | 0.79 | 0.57 | 1.09 | 0.15 |
| | | PMID: 33542074 | | | | | | |
| 5879 | RAC1 | PMID: 32782617 | 2.45 | 0.02* | 1.02 | 1.01 | 1.04 | 0.003* |
| 10413 | YAP1 | PMID: 33664454 | 1.34 | 0.2 | 1.01 | 0.97 | 1.05 | 0.58 |
| 7474 | WNT5A | https://doi.org/10.1097/ | 0.71 | 0.49 | 1.02 | 1.00 | 1.03 | 0.02* |
| | | JU0000000000002526.02 | | | | | | |
| 4853 | NOTCH2 | PMID: 30940724 | 0.66 | 0.52 | 0.99 | 0.92 | 1.06 | 0.74 |
| 3551 | IKBKB | PMID: 33542074 | −0.47 | 0.64 | 1.01 | 0.96 | 1.06 | 0.78 |
| 8644 | AKR1C3 | PMID: 25649766 | −0.27 | 0.79 | 1.02 | 0.99 | 1.06 | 0.17 |
| 2535 | FZD2 | https://doi.org/10.1097/ | 0.01 | 1 | 0.96 | 0.66 | 1.40 | 0.84 |
| | | JU0000000000002526.02 | | | | | | |

*significant p-value <0.05

TABLE 8
Comparison to known genomic markers related with response to Enzalutamide

| | Progression | | Deletion (both Shallow and Deep) | | | | | Gain and Amplification | |
|---|---|---|---|---|---|---|---|---|---|
| Human | related | Publication | Fisher | Hazard | lower_ | upper_ | Cox | Fisher | Hazard |
| entrez | markers | PubMed Id | exact p | ratio | 95 | 95 | p | exact p | ratio |
| 1105 | CHD1 | PMID: 32220301 | 0.38 | 1.05 | 0.38 | 2.92 | 0.91 | 1 | 0.64 |
| 54894 | RNF43 | PMID: 32727885 | 0.49 | 1.27 | 0.28 | 5.74 | 0.76 | 1 | 0.42 |
| | | Gain and Amplification | | | Diploid | | | | |
| | Human | lower_ | upper_ | Cox | Fisher | Hazard | lower_ | upper_ | Cox |
| | entrez | 95 | 95 | p | exact p | ratio | 95 | 95 | p |
| | 1105 | 0.08 | 4.94 | 0.67 | 0.2 | 1.07 | 0.4 | 2.85 | 0.88 |
| | 54894 | 0.05 | 3.22 | 0.4 | 0.62 | 1.34 | 0.38 | 4.71 | 0.64 |

### Comparison to Gene-Centric Computational Methods

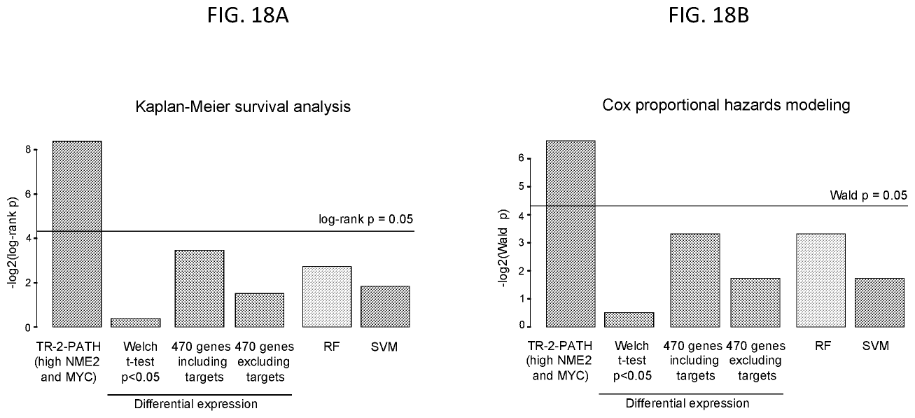

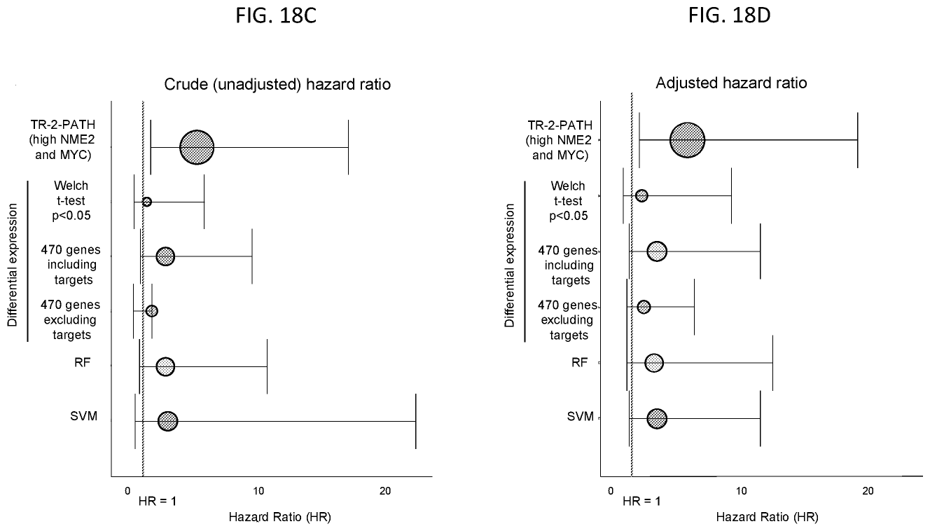

To evaluate if TR-2-PATH mechanism-centric predictions (activity levels of NME2 TR and MYC pathway) outperform predictive ability of commonly used gene-centric methods, we compared TR-2-PATH to differential expression analyses, Random (survival) Forest (RF), and Support Vector Machine (SVM) methods all utilized on the Enzalutamide-specific Abida et al. cohort. For differential gene expression analysis, we considered genes that were differentially expressed between the three phenotypes (Intact, EnzaSens, and EnzaRes) in the mining step I and considered genes at (i) Welch t-test p-value<0.05; (ii) top 470 differentially expressed genes (comparable to the total number of target genes and pathway genes used for activity estimation) and not excluding target/pathway genes from NME2 TR and MYC pathway; (iii) top 470 differentially expressed genes, excluding target/pathway genes from NME2 TR and MYC pathway. For RF and SVM analysis, we utilized 470 genes from (iii) to avoid overfitting and then selected top 10 most significant genes/features from the outputs. Final gene list from each of these analyses was subjected to Kaplan-Meier survival analysis and Cox proportional hazards model analysis (crude and adjusted for age and Gleason), which did not show a significant association with Enzalutamide-associated disease progression using log-rank test, Wald test, or crude/adjusted hazards models (FIGS. 18A-18D). Such analysis demonstrates that mechanisms identified by TR-2-PATH (NME2 TR and MYC pathway) have significant advantage in predicting the risk of Enzalutamide resistance, compared to commonly used gene-centric methods.

### Targeting MYC and NME2 is Beneficial in Enzalutamide Resistant Conditions

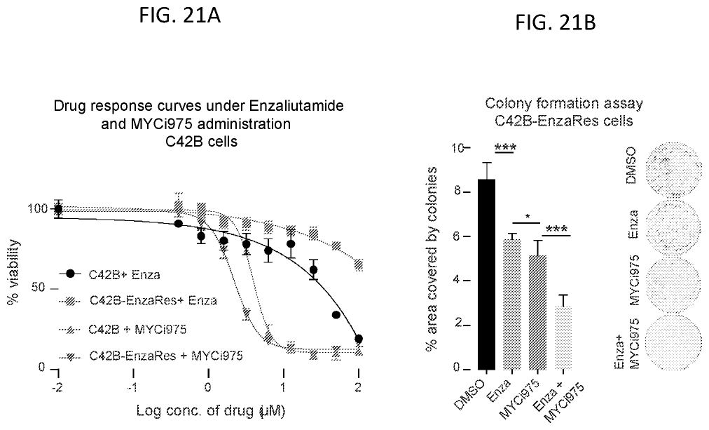

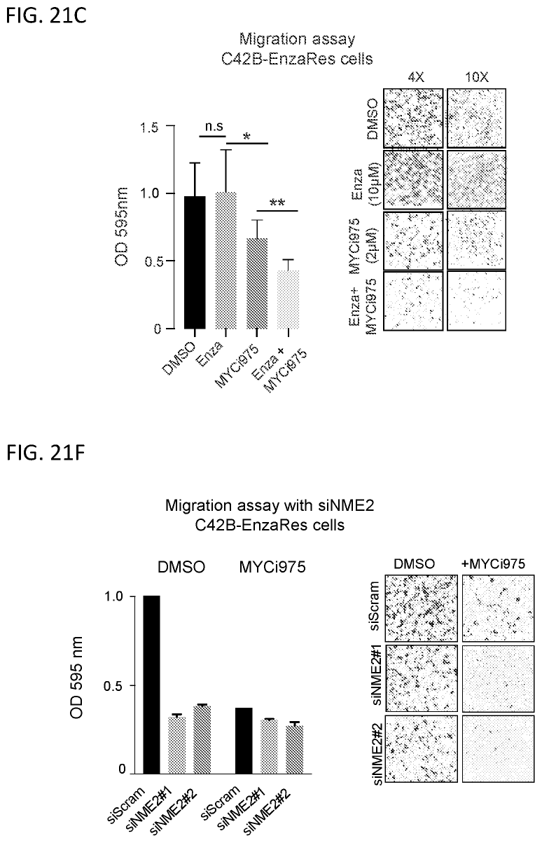

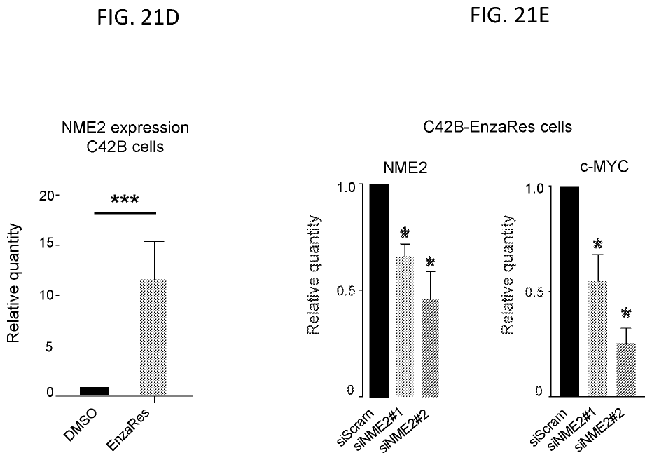

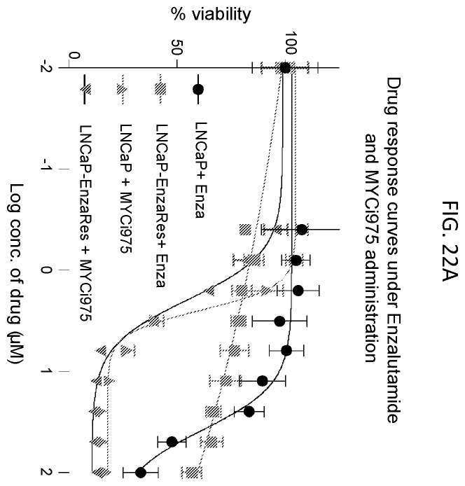

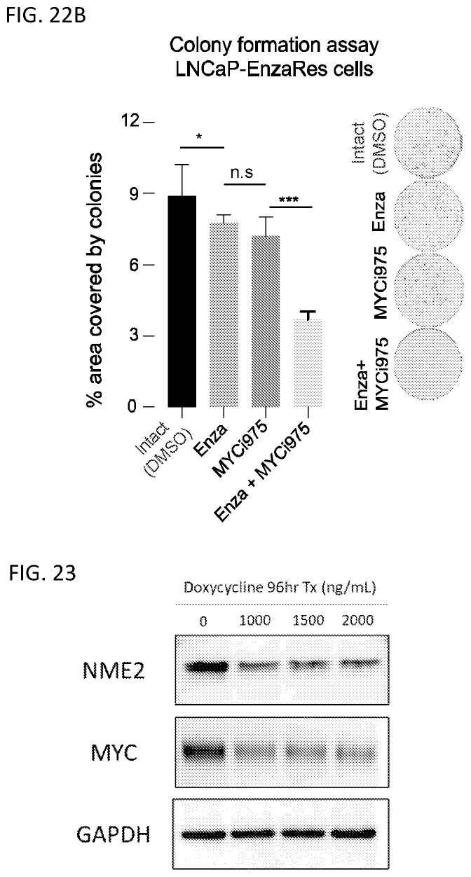

Given that NME2 and MYC are upregulated in both patients that are at risk of Enzalutamide resistance and patients that fail Enzalutamide, we experimentally evaluated the benefits of therapeutic targeting of MYC and NME2 in similar experimental conditions. For this, we utilized LNCaP and C42B cell lines, as our experimental systems in Enzalutamide-naïve and Enzalutamide-resistant (EnzaRes) conditions (see Methods). To target MYC we used MYCi975, a small molecule that directly inhibits MYC activity, developed by Han et al. (Cancer Cell 36:483-497, 2019). To understand the dose-dependent effect of MYCi975 in Enzalutamide-naïve and Enzalutamide-resistant conditions, we performed dose-response assays in both LNCaP and C42B cell lines (FIG. 21A, FIG. 22A, see Methods) with varying doses of both drugs. This analysis demonstrated a striking reduction of the cell viability when treated with MYCi975 both in Enzalutamide-naïve and Enzalutamide-resistant conditions in a dose-dependent manner (FIGS. 21A, 22A).

Next, we utilized identified sub-IC₅₀ concentrations of Enzalutamide (10 μM) alone, MYCi975 (2 μM) alone, or Enzalutamide and MYCi975 in combination, to perform colony formation assay using Enzalutamide-resistant LNCaP and C42B cells. Inhibition of MYC using MYCi975 reduced the colony formation of LNCaP-EnzaRes cells (one-tailed Welch t-test p-value=0.013, FIG. 22B) and C42B-EnzaRes cells (one-tailed Welch t-test p-value=3.98E−6, FIG. 13B), compared to Intact (DMSO). Interestingly, the colony formation ability was significantly reduced when MYCi975 and Enzalutamide were administered in combination on LNCaP-EnzaRes cells (one-tailed Welch t-test p-value=6.39E−5 FIG. 18B) and C42B-EnzaRes (one-tailed Welch t-test p-value=4.7E−8, FIG. 21B) compared to Intact (DMSO).

To evaluate the impact of Enzalutamide alone, MYCi975 alone, or in combination on the migratory capacity of Enzalutamide-resistant cells, we performed Boyden chamber-based in vitro migration assay using C42B-EnzaRes cells (LNCaP cells do not migrate) and observed a significant reduction in cell migration when treated with MYCi975 (one-tailed Welch t-test p-value=0.02, FIG. 21C) and even greater reduction when treated with a combination of MYCi975 and Enzalutamide (one-tailed Welch t-test p-value=0.003, FIG. 21C).

Next, to evaluate the effect of NME2 silencing on MYC expression, we first evaluated the expression of NME2 in EnzaRes compared to Intact in C42B cells, which showed elevated levels of NME2 in C42B-EnzaRes cells (FIG. 21D, one-tailed Welch t-test p-value=0.0005). NME2 knockdown in C42B-EnzaRes cells using two different siRNAs (FIG. 21E), demonstrated a significant reduction in expression of NME2 (FIG. 21E, left) and MYC (FIG. 21E, right), supported by the previously identified NME2 upstream regulation of MYC.

In addition, to evaluate if silencing of NME2 could enhance the effect of MYC inhibition on cells' metastatic potential, we performed Boyden chamber-based in vitro migration assay using C42B-EnzaRes cells, which demonstrated that the cell migration was further reduced when NME2 knockdown was added to MYCi975 administration (FIG. 21F).

Finally, C4-2B cells expressing doxycycline-inducible shRNA targeting NME2 were generated using the targeting sequence GAAATCAGCCTATGGTTTAAG (SEQ ID NO: 9). Cells were treated with 0-2000 ng/mL doxycycline for 96 hours and NME2 and MYC expression were evaluated by Western blot. This confirmed that NME2 knockdown suppresses MYC expression (FIG. 23).

Taken together, our results using relevant pre-clinical models of Enzalutamide resistance indicate that therapeutic targeting of MYC is beneficial in Enzalutamide-resistant conditions and MYC inhibition could be combined with concurrent Enzalutamide administration for improved efficacy. Further, apart from direct inhibition of MYC, its indirect inhibition via NME2 knockdown enhances MYC-related therapeutic targeting. We propose that MYC-centered therapeutic targeting could be an alternative therapy for patients at risk of Enzalutamide-resistance and/or salvage therapy for patients that failed Enzalutamide treatment.

### EXAMPLE 17—EXAMPLE COMPUTING SYSTEM

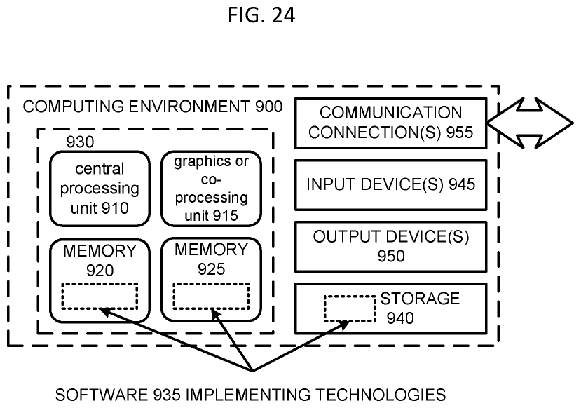

FIG. 24 illustrates a generalized example of a suitable computing system 900 in which any of the described technologies may be implemented. The computing system 900 is not intended to suggest any limitation as to scope of use or functionality, as the innovations may be implemented in diverse computing systems, including special-purpose computing systems. In practice, a computing system can comprise multiple networked instances of the illustrated computing system.

With reference to FIG. 24, the computing system 900 includes one or more processing units 910, 915 and memory 920, 925. In FIG. 24, this basic configuration 930 is included within a dashed line. The processing units 910, 915 execute computer-executable instructions. A processing unit can be a central processing unit (CPU), processor in an application-specific integrated circuit (ASIC), or any other type of processor. In a multi-processing system, multiple processing units execute computer-executable instructions to increase processing power. For example, FIG. 24 shows a central processing unit 910 as well as a graphics processing unit or co-processing unit 915. The tangible memory 920, 925 may be volatile memory (e.g., registers, cache, RAM), non-volatile memory (e.g., ROM, EEPROM, flash memory, etc.), or some combination of the two, accessible by the processing unit(s). The memory 920, 925 stores software 935 implementing one or more innovations described herein, in the form of computer-executable instructions suitable for execution by the processing unit(s).

A computing system may have additional features. For example, the computing system 900 includes storage 940, one or more input devices 945, one or more output devices 950, and one or more communication connections 955. An interconnection mechanism (not shown) such as a bus, controller, or network interconnects the components of the computing system 900. Typically, operating system software (not shown) provides an operating environment for other software executing in the computing system 900, and coordinates activities of the components of the computing system 900.

The tangible storage 940 may be removable or non-removable, and includes magnetic disks, magnetic tapes or cassettes, CD-ROMs, DVDs, flash drives, external hard drives, or any other medium which can be used to store information in a non-transitory way and which can be accessed within a computing system. The storage 940 stores instructions for the software 935 implementing one or more innovations described herein.

The input device(s) 945 may be a touch input device such as a keyboard, mouse, pen, or trackball, a voice input device, a scanning device, or another device that provides input to the computing system 900. For video encoding, the input device(s) 945 may be a camera, video card, TV tuner card, or similar device that accepts video input in analog or digital form, or a CD-ROM or CD-RW, flash drive, or external hard drive, that reads video samples into the computing system 900. The output device(s) 945 may be a display, printer, speaker, CD-writer, or another device that provides output from the computing system 900.

The communication connection(s) 955 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor (e.g., that is ultimately implemented on a hardware processor). Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system.

For the sake of presentation, the detailed description uses terms like “determine” and “use” to describe computer operations in a computing system. These terms are high-level abstractions for operations performed by a computer and should not be confused with acts performed by a human being. The actual computer operations corresponding to these terms vary depending on implementation.

### EXAMPLE 18—EXAMPLE CLOUD COMPUTING ENVIRONMENT

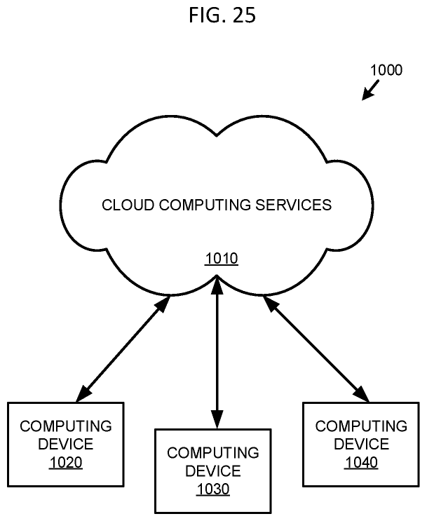

FIG. 25 depicts an example cloud computing environment 1000 in which the described technologies can be implemented, including, e.g., the systems of the drawings described herein. The cloud computing environment 1000 comprises cloud computing services 1010. The cloud computing services 1010 can comprise various types of cloud computing resources, such as computer servers, data storage repositories, networking resources, etc. The cloud computing services 1010 can be centrally located (e.g., provided by a data center of a business or organization) or distributed (e.g., provided by various computing resources located at different locations, such as different data centers and/or located in different cities or countries).

The cloud computing services 1010 are utilized by various types of computing devices (e.g., client computing devices), such as computing devices 1020, 1030, and 1040. For example, the computing devices (e.g., 1020, 1030, and 1040) can be computers (e.g., desktop or laptop computers), mobile devices (e.g., tablet computers or smart phones), or other types of computing devices. For example, the computing devices (e.g., 1020, 1030, and 1040) can utilize the cloud computing services 1010 to perform computing operations (e.g., data processing, data storage, and the like).

In practice, cloud-based, on-premises-based, or hybrid scenarios can be supported.

### EXAMPLE 19—EXAMPLE COMPUTER-READABLE MEDIA

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### EXAMPLE 20—EXAMPLE IMPLEMENTATIONS

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### EXAMPLE 21—EXAMPLE COMPUTER-EXECUTABLE IMPLEMENTATION

Any of the methods described herein can be performed by computer-executable instructions (e.g., causing a computing system to perform the method, when executed) stored in one or more computer-readable media (e.g., storage or other tangible media) or stored in one or more computer-readable storage devices. Such methods can be performed in software, firmware, hardware, or combinations thereof. Such methods can be performed at least in part by a computing system (e.g., one or more computing devices).

Such acts of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

In any of the technologies described herein, the illustrated actions can be described from alternative perspectives while still implementing the technologies. For example, “receiving” can also be described as “sending” for a different perspective.

### EXAMPLE 22—EXAMPLE ALTERNATIVES

The technologies from any example can be combined with the technologies described in any one or more of the other examples. In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of identifying treatment-response signatures, comprising:
(i) receiving a gene expression dataset from
(a) one or more subjects having a condition or disease; or
(b) one or more cell cultures representative of the condition or disease;
(ii) identifying one or more transcriptional regulatory programs and one or more molecular pathways that are enriched in the gene expression dataset;
(iii) determining one or more relationships between the one or more transcriptional regulatory programs and the one or more molecular pathways enriched in the gene expression dataset, wherein the determining generates at least one network for the gene expression dataset; and
(iv) identifying one or more molecular pathways and/or one or more transcriptional regulatory programs in the network that comprise one or more genes that are:
relatively upregulated or downregulated in a gene expression dataset from a sample that has not received the treatment as compared to a sample that is sensitive to the treatment,
relatively upregulated or downregulated in a gene expression dataset from a sample that has not received the treatment as compared to a sample that is resistant to the treatment, or
relatively upregulated or downregulated in a gene expression dataset from a sample that is sensitive to the treatment as compared to a sample that exhibits resistance to the treatment;
wherein the identifying generates a treatment-response signature.

2. The method of claim 1, comprising identifying one or more molecular pathways and/or one or more transcriptional regulatory programs in the network that comprise one or more genes that are
(a) relatively upregulated in a gene expression dataset from a sample that has not received the treatment as compared to a sample that is sensitive to the treatment, relatively downregulated in a gene expression dataset from a sample that is sensitive to the treatment as compared to a sample that has not received the treatment and/or to a sample that exhibits resistance to the treatment, and relatively upregulated in a gene expression dataset from a sample that exhibits resistance to the treatment as compared to a sample that is sensitive to the treatment; or
(b) relatively downregulated in a gene expression dataset from a sample that has not received the treatment as compared to a sample that is sensitive to the treatment, relatively upregulated in a gene expression dataset from a sample that is sensitive to the treatment as compared to a sample that has not received the treatment and/or to a sample that exhibits resistance to the treatment, and relatively downregulated in a gene expression dataset from a sample that exhibits resistance to the treatment as compared to a sample that is sensitive to the treatment.

3. The method of claim 1, wherein
the one or more subjects having the condition or disease have not received the treatment;
the one or more subjects having the condition or disease have received the treatment;
the one or more cell cultures representative of the condition or disease have not received the treatment;
the one or more cell cultures representative of the condition or disease have received the treatment;
some of the one or more cell cultures representative of the condition or disease have received the treatment, and some of the one or more cell cultures representative of the condition or disease have not received the treatment; or
some of the one or more subjects having the condition or disease have received the treatment, and some of the one or more subjects having the condition or disease have not received the treatment.

4. The method of claim 1, wherein
the identifying one or more transcriptional regulatory programs that are enriched in the gene expression dataset comprises performing a transcriptional regulatory program analysis using the gene expression dataset; and
the identifying one or more molecular pathways that are enriched in the gene expression dataset comprises performing a molecular pathway enrichment analysis using the gene expression dataset.

5. The method of claim 4, wherein:
(a) the performing a molecular pathway enrichment analysis using the gene expression dataset comprises:
(i) measuring expression of genes of the molecular pathways in the gene expression dataset;
(ii) measuring expression of genes of the molecular pathways in a reference gene expression set; and
(iii) comparing the expression of the genes of (i) and (ii), wherein the comparing generates a set of molecular pathways enriched in the gene expression dataset; and/or
(b) the performing a transcriptional regulatory program analysis comprises:
(i) measuring expression of genes of the transcriptional regulatory programs in the gene expression dataset;
(ii) measuring expression of genes of the transcriptional regulatory programs in a reference gene expression set; and
(iii) comparing the expression of the genes of (i) and (ii), wherein the comparing generates a set of transcriptional regulatory programs enriched in the gene expression dataset.

6. The method of claim 5, wherein the reference gene expression set comprises genes ranked by differential expression between at least two samples.

7. The method of claim 6, wherein the at least two samples comprise at least one sample that has resistance to the treatment and at least one sample that has sensitivity to the treatment.

8. The method of claim 1, wherein identifying one or more transcriptional regulatory programs and one or more molecular pathways enriched in the gene expression dataset comprises removing transcriptional regulatory programs and molecular pathways from the network that are enriched with a p-value greater than 0.001 or a false discovery rate-corrected p-value greater than 0.05.

9. The method of claim 1, wherein the determining one or more relationships between the one or more transcriptional regulatory programs and the one or more molecular pathways enriched in the gene expression dataset comprises:
determining an activity vector for each molecular pathway;
determining an activity vector for each transcriptional regulatory program;
identifying one or more statistically significant relationships between each transcriptional regulatory program and each molecular pathway; and
incorporating each of the one or more statistically significant relationships into the network as an edge of the network.

10. The method of claim 9, wherein identifying one or more statistically significant relationships between each transcriptional regulatory program and each molecular pathway comprises performing pairwise comparisons between each molecular pathway activity vector and each transcriptional regulatory network activity vector.

11. The method of claim 10, wherein the pairwise comparisons are performed using linear regression; and/or
wherein each transcriptional regulatory program activity vector is an independent variable in the regression analysis and each molecular pathway activity vector is a dependent variable in the regression analysis.

12. The method of claim 8, wherein incorporating each of the one or more statistically significant relationships into the network as an edge of the network comprises:
performing multiple hypothesis testing to identify each transcriptional regulatory program that has a relationship with each molecular pathway;
calculating a false discovery rate; and
incorporating each relationship into the network as an edge when the false discovery rater for the relationship is less than 0.05.

13. The method of claim 11, where a slope of the regression indicates a directionality of the relationship between the molecular pathway and the transcriptional regulatory program, wherein a positive slope of the regression indicates a positive relationship between the molecular pathway and the transcriptional regulatory program, and a negative slope of the regression indicates a negative relationship between the molecular pathway and the transcriptional regulatory program.

14. The method of claim 9, further comprising determining a weight of each edge incorporated into the network.

15. The method of claim 14, wherein determining the weight of each edge incorporated into the network comprises a bootstrap analysis, wherein the weight of each edge is the number of times the edge is identified and has the same directionality in the network and across a number of bootstrapped networks.

16. The method of claim 1, wherein datapoints of the gene expression dataset are normalized before identifying molecular pathways and transcriptional regulatory programs enriched in the gene expression dataset.

17. The method of claim 9, wherein generating the treatment-response signature further comprises:
(i) identifying molecular pathways and/or transcriptional regulatory programs that are significantly negatively enriched between the gene expression dataset from the sample that has not received the treatment and the gene expression dataset from the sample that is sensitive to the treatment;
(ii) identifying molecular pathways and/or transcriptional regulatory programs that are significantly positively enriched between the gene expression dataset from the sample that is sensitive to the treatment and the gene expression dataset from the sample that is resistant to the treatment;
(iii) comparing expression of genes in the molecular pathways and transcriptional programs identified in (i) and the molecular pathways and transcriptional programs identified in (ii) with expression of genes in the molecular pathways and transcriptional programs in the network;
(iv) performing gene set enrichment analyses comparing the molecular pathways and transcriptional programs identified in (i) to the molecular pathways and transcriptional programs identified in (ii);
(v) selecting molecular pathways and transcriptional regulatory networks that are significantly enriched in both (i) and (ii);
(vi) identifying at least one non-collinear latent variable using the activity vector for each selected molecular pathway and the activity vector for each selected transcriptional regulatory program;
(vii) identifying and clustering collinear transcriptional regulatory programs using the at least one non-collinear latent variable;
(viii) calculating degree of closeness between each transcriptional regulatory program or cluster of collinear regulatory programs and each non-collinear latent variable, and ranking each transcriptional regulatory program or cluster of collinear regulatory programs by degree of closeness;
(ix) calculating degree of closeness between each transcriptional regulatory program or cluster of collinear regulatory programs and each molecular pathway, and ranking each transcriptional regulatory program or cluster of collinear regulatory programs by degree of closeness;
(x) determining the robustness of the relationship between each transcriptional regulatory program or cluster of collinear regulatory programs and each molecular pathway using the calculated edge weights for each relationship, and ranking each transcriptional regulatory program or cluster of collinear regulatory programs by robustness of the relationship; and
(xi) combining the rankings of (viii), (ix), and (x) and selecting the transcriptional regulatory program having the closest relationship to each molecular pathway, wherein the treatment-response signature comprises the selected molecular pathways and transcriptional regulatory networks.

18. The method of claim 17, wherein identifying non-collinear latent variables and calculating the degree of closeness between each transcriptional regulatory program and each non-collinear latent variable comprise performing a partial least square regression analysis.

19. The method of claim 17, wherein combining the rankings of (vii)-(ix) and selecting the transcriptional regulatory program having the closest relationship to each molecular pathway comprises calculating the rank product of (vii)-(ix) using the geometric mean.

20. The method of claim 1, wherein the sample that has not received the treatment, the sample that is sensitive to the treatment, and the sample that has resistance to the treatment are samples from the same subject, different subjects, or any combination thereof.

21. The method of claim 1, wherein the method is a computer implemented method.

22. A treatment-response signature identification system, comprising:
one or more processors; and
memory coupled to the one or more processors, wherein the memory comprises computer-executable instructions causing the one or more processors to perform a process comprising:
(i) receiving a gene expression dataset from
(a) one or more subjects having a condition or disease; or
(b) one or more cell cultures representative of the condition or disease;
(ii) identifying one or more transcriptional regulatory programs and one or more molecular pathways that are enriched in the gene expression dataset;
(iii) determining one or more relationships between the one or more transcriptional regulatory programs and the one or more molecular pathways enriched in the gene expression dataset, wherein the determining generates at least one network for the gene expression dataset; and
(iv) identifying one or more molecular pathways and/or one or more transcriptional regulatory programs in the network that comprise one or more genes that are:
relatively upregulated or downregulated in a gene expression dataset from a sample that has not received the treatment as compared to a sample that is sensitive to the treatment,
relatively upregulated or downregulated in a gene expression dataset from a sample that has not received the treatment as compared to a sample that is resistant to the treatment, or
relatively upregulated or downregulated in a gene expression dataset from a sample that is sensitive to the treatment as compared to a sample that exhibits resistance to the treatment;
wherein the identifying generates a treatment-response signature.

23. One or more computer-readable media having encoded thereon computer-executable instructions that, when executed, cause a computing system to perform a treatment-response signature identification method, comprising:
(i) receiving a gene expression dataset from
(a) one or more subjects having a condition or disease; or
(b) one or more cell cultures representative of the condition or disease;
(ii) identifying one or more transcriptional regulatory programs and one or more molecular pathways that are enriched in the gene expression dataset;
(iii) determining one or more relationships between the one or more transcriptional regulatory programs and the one or more molecular pathways enriched in the gene expression dataset, wherein the determining generates at least one network for the gene expression dataset; and
(iv) identifying one or more molecular pathways and/or one or more transcriptional regulatory programs in the network that comprise one or more genes that are:
relatively upregulated or downregulated in a gene expression dataset from a sample that has not received the treatment as compared to a sample that is sensitive to the treatment,
relatively upregulated or downregulated in a gene expression dataset from a sample that has not received the treatment as compared to a sample that is resistant to the treatment, or
relatively upregulated or downregulated in a gene expression dataset from a sample that is sensitive to the treatment as compared to a sample that exhibits resistance to the treatment;
wherein the identifying generates a treatment-response signature.


SEQUENCE LISTING (9 sequences)

SEQ ID NO: 1 - DNA, 21 bp, synthetic construct
  source 1..21: mol_type = other DNA
cctggtgctccatgaggagac

SEQ ID NO: 2 - DNA, 22 bp, synthetic construct
  source 1..22: mol_type = other DNA
cagactctgaccttttgccagg

SEQ ID NO: 3 - DNA, 22 bp, synthetic construct
  source 1..22: mol_type = other DNA
tcttgtcgtcttcggaaatgtt

SEQ ID NO: 4 - DNA, 21 bp, synthetic construct
  source 1..21: mol_type = other DNA
aagcctctccttcctcctgta

SEQ ID NO: 5 - DNA, 22 bp, synthetic construct
  source 1..22: mol_type = other DNA
aggattccgccttgttggtctg

SEQ ID NO: 6 - DNA, 22 bp, synthetic construct
  source 1..22: mol_type = other DNA
cggcaaagaatggacggtcctt

SEQ ID NO: 7 - RNA, 19 nt, synthetic construct
  source 1..19: mol_type = other RNA
aataagaggtggacacaac

SEQ ID NO: 8 - RNA, 19 nt, synthetic construct
  source 1..19: mol_type = other RNA
ctgaagaacacctgaagca

SEQ ID NO: 9 - DNA, 21 bp, synthetic construct
  source 1..21: mol_type = other DNA
gaaatcagcctatggtttaag